(19) United States
(12) Patent Application Publication
(10) Pub. No.: US 2010/0303758 A1
(43) Pub. Date: Dec. 2, 2010

(54) Novel Glucocorticoid Receptor Agonists

(76) Inventors: Paul Alan Glossop, Sandwich (GB); David Simon Millan, Sandwich (GB); David Anthony Price, Mystic, CT (US)

Correspondence Address: PFIZER INC.;PATENT DEPARTMENT, Bld 114 M/S 9114, EASTERN POINT ROAD, GROTON, CT, 06340, US

(21) Appl. No.: 12/787,444

(22) Filed: May 26, 2010

Related U.S. Application Data
(60) Provisional application No. 61/182,266, filed on May 29, 2009.

(51) Int. Cl. A61K 38/20; C07J 3/00; A61K 31/56; A61K 31/522; A61K 31/353; A61K 38/43; A61K 39/395; A61K 38/00; A61K 38/45; A61K 31/4406; A61P 17/00; A61P 29/00; A61P 19/02; A61P 11/00; A61P 1/02; A61P 27/00

(52) U.S. Cl. 424/85.2; 552/610; 514/180; 514/263.34; 514/456; 424/94.1; 424/130.1; 424/94.5; 514/356; 514/17.4; 514/20.1; 514/20.4

(57) ABSTRACT

This invention relates to novel glucocorticoid receptor agonists of formula (I):

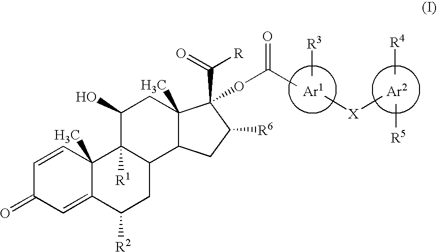

and to processes and intermediates for their preparation. The present invention also relates to pharmaceutical compositions containing these compounds, to their combination with one or more other therapeutic agents, as well as to their use for the treatment of a number of inflammatory and allergic diseases, disorders and conditions.

This present invention relates to novel glucocorticoid receptor agonists and to pharmaceutically acceptable salts thereof or pharmaceutically acceptable solvates of said glucocorticoid receptor agonists or salts, processes and intermediates for their preparation. The present invention also relates to pharmaceutical compositions containing these compounds, to their combination with one or more other therapeutic agents, as well as to their use for the treatment of a number of inflammatory and allergic diseases, disorders and conditions.

Glucocorticoid receptor agonists are potent anti-inflammatory drugs that are indispensable for the treatment of a broad array of inflammatory and immunological disorders. The first compounds introduced into therapy were derived from the natural corticosteroid hydrocortisone. First structural modifications of the core molecule aimed at the increase in selectivity to the glucocorticoid over the mineralo-corticoid receptor. Based on a better understanding of structure-activity relationships, the next generation of compounds displayed higher receptor affinities and thus higher efficacy. For topically applied glucocorticoids, further progress was achieved by drug targeting e.g. by inhalation or skin application of corticosteroid preparations. Recent developments focused on the best possible reduction of adverse effects by introducing metabolically labile functional groups into the active molecule to minimize systemic exposure after topical application. High affinity to the therapeutic target tissue was recognized as a property that enhances on-target efficacy and duration of action while limiting off-target systemic effects by slowing redistribution into the systemic circulation.

Glucocorticoid receptor agonists are used in the management of inflammatory and allergic conditions, e.g. asthma, obstructive airway diseases, rhinitis, inflammatory bowel disease, psoriasis, eczema etc. Examples of already marketed glucocorticoids include:

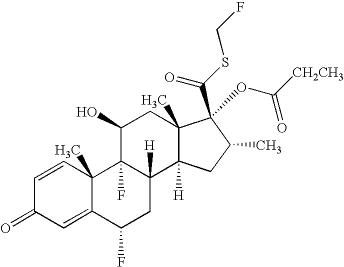

Fluticasone propionate (Flovent™, Flonase™)

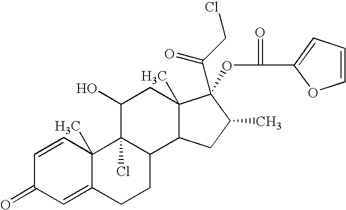

### Mometasone (Nasonex™, Asmanex™)

These compounds bind to and activate glucocorticoid receptors in a wide range of cell types. The activated receptor binds to glucocorticoid response elements in the nucleus activating or inhibiting transcription of genes that have key regulatory functions. In particular these compounds are efficacious in inflammatory diseases by preventing the recruitment of inflammatory leukocytes, such as eosinophils and neutrophils to sites of inflammation and also inhibiting the formation and release of inflammatory mediators from leukocytes and tissue cells.

Since the marketing of the first corticosteroids, numerous corticosteroids have been proposed having different structures such as for example the compounds as described in WO 02/00679 of formula:

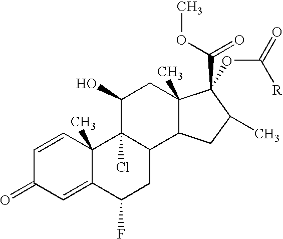

wherein R is a monovalent cyclic organic group having 3 to 15 atoms in the ring system.

Other examples include the compounds as described in WO 2002/12266 of formula:

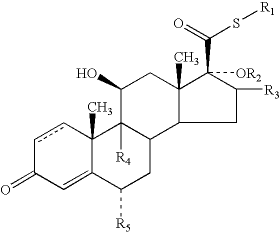

wherein R₁ is an alkyl or an haloalkyl. R₂ represents —C(═O)-aryl or —C(═O)-heteroaryl, R₃ is H, methyl or methylene and R₄ and R₅ are the same or different and each represents H or halogen.

Further examples include the compounds as described in WO 2005/005451 of formula:

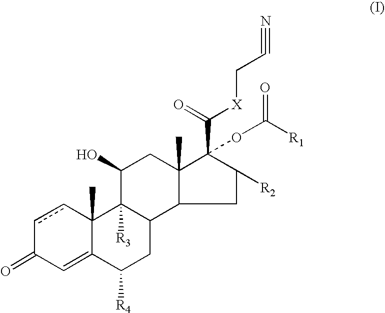

wherein X is O or S, R₁ may represent a (un)substituted aryl or heteroaryl, R₂ is H, methylor methylene and R₃ and R₄ are the same or different and each represents H, halogen or a methyl group.

Finally, other examples include the compounds as described in WO 2007/099548 of formula:

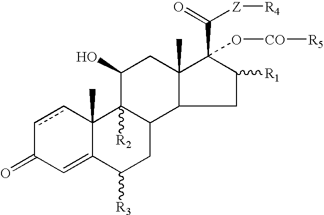

wherein [symbol] may be a double bond; Z represents O or S; R₄ is selected from:

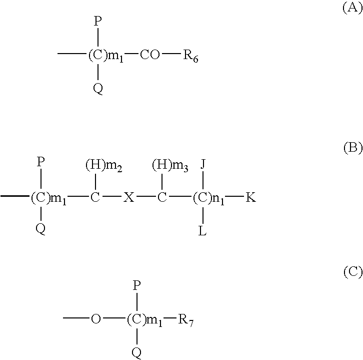

with the proviso that when R₄ represents moiety (C) then Z is 8, R₁ is H, methyl or methylene; R₂ and R₃ are the same or different and each independently represents H, halogen or methyl; and R₅ may be e.g. an aryl or an heterocyclic ring which is unsubstituted or substituted by halogen, OH, (C₁-C₃)alkyl, —O—(C₁-C₃)alkyl, (C₃-C₁₃)cycloalkyl wherein the alkyl or cycloalkyl groups can optionally contain 1 or more unsaturation(s) and or can have one or more heteroatom incorporated therein and optionally in each case have one or more H atoms replaced by halogen, OH, (C₁-C₃)alkyl, —O—(C₁-C₃)alkyl or (C₃-C₁₃)cycloalkyl.

However, there is still a need for improved glucocorticoid receptor agonists that would have the most appropriate pharmacological profile, for example in terms of potency, duration of action, therapeutic index, pharmacokinetics, drug/drug interactions and/or side effects.

In this context, there is provided a compound of formula (I):

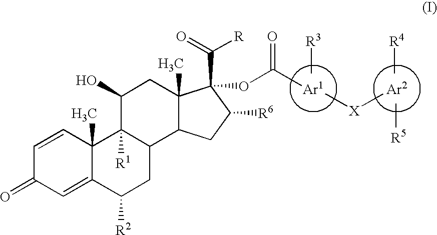

or a pharmaceutically acceptable salt thereof or a pharmaceutically acceptable solvate of said compound or salt, wherein:
R¹ and R² are independently of each other selected from H, F, Cl and methyl;
R is selected from —CH₂—OH, —O—CH₂—CN, —S—CH₂—CN, —O—CH₂F, —S—CH₂F, —O—CH₂Cl and —S—CH₂Cl;
X is a direct bond or represents a moiety selected from —O—, —S—, —CH₂—S—, —S—CH₂—, —CH₂—, —O—CH₂, and —CH₂—O—;
Ar¹ represents a phenyl or a pyridine;
Ar² represents an aryl group selected from phenyl, pyridine, pyridazine, pyrazine and pyrimidine;

### R³ is H or OH;

### R⁴ is H or OH; and

R⁵ is selected from H, CN, halogen, (C₁-C₄)alkyl, —S—(C₁-C₄)alkyl, —CONR⁷R⁸, —SO₂NR⁷R⁸ and NHSO₂CH₃;

### R⁶ is H or CH₃; and

R⁷ and R⁸ are the same or are different and are independently selected from H and (C₁-C₄)alkyl.

Unless otherwise defined herein, scientific and technical terms used in connection with the present invention have the meanings that are commonly understood by those of ordinary skill in the art.

The term “halogen” denotes a halogen atom selected from the group consisting of fluoro, chloro, bromo and iodo. More preferably, halogen denotes a fluoro or a chloro atom.

The term “(C₁-C₄)alkyl”, alone or in combination, means an acyclic, saturated hydrocarbon group of the formula CₙH₂ₙ₊₁ which may be linear or branched and which contains 1, 2, 3 or 4 carbon atoms. Examples of such groups include methyl, ethyl, n-propyl, isopropyl, n-butyl, isobutyl, sec-butyl and tert-butyl.

The subgroups of compounds of formula (I) containing the following substituents, or combinations of the following substituents, are preferred:
R¹ and R² are independently selected from H, F and Cl; more preferably R¹ is F or Cl and R² is H or F; even more preferably, R¹ is F and R² is H or F.
R is selected from —CH₂—OH, —O—CH₂—CN, —S—CH₂—CN, —S—CH₂F, —O—CH₂F and —S—CH₂Cl; more preferably R is selected from —O—CH₂—CN, —S—CH₂—CN, —S—CH₂F and —O—CH₂F; even more preferably, R is —O—CH₂F.
Ar¹ is phenyl and Ar² is selected from phenyl, pyridine, pyridazine and pyrazine and pyrimidine; more preferably Ar¹ is phenyl and Ar² is phenyl or pyridine; even more preferably Ar¹ and Ar² are both phenyl.
X is a direct bond or represents a moiety selected from —O—, —S—, —CH₂—S—, —S—CH₂—, —CH₂— and —O—CH₂; more preferably, X is —O—.
R³ is H.
R⁴ is preferably OH and in that case, said hydroxyl group is preferably in a meta or para position relative to X.
R⁵ is selected from H, CN, halogen, —S—(C₁-C₄)alkyl, —CONR⁷R⁸, wherein R⁷ and R⁸ are the same or different and are independently selected from H and CH₃; more preferably R⁵ is selected from H, CN, F, Cl, —S—CH₃, —CONH₂, and —CON(CH₃)₂; even more preferably R⁵ is selected from H, F, Cl and —S—CH₃; Still more preferably, R⁵ is H, Cl or —S—CH₃; Still more preferably, R⁵ is Cl.

According to another embodiment, the sub-group of glucocorticoid receptor agonists of formula (Ia):

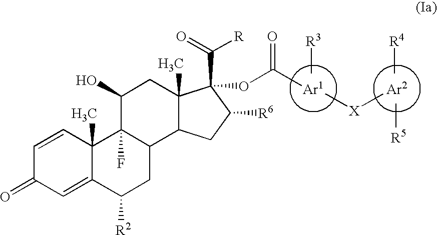

or a pharmaceutically acceptable salt thereof or a pharmaceutically acceptable solvate of said compound or salt, wherein:

### R² is H or F;

R is selected from —CH₂—OH, —O—CH₂—CN, —S—CH₂—CN, —O—CH₂F, —S—CH₂F, —O—CH₂Cl and —S—CH₂Cl;
X is a direct bond or represents a moiety selected from —O—, —S—, —CH₂—S—, —S—CH₂—, —CH₂—, —O—CH₂, and —CH₂—O—;
Ar¹ represents a phenyl or a pyridine;
Ar² represents an aryl group selected from phenyl, pyridine, pyridazine, pyrazine and pyrimidine;

### R³ is H or OH;

### R⁴ is H or OH; and

R⁵ is selected from H, CN, halogen, (C₁-C₄)alkyl, —S—(C₁-C₄)alkyl, —CONR⁷R⁸, —SO₂NR⁷R⁸ and NHSO₂CH₃;

### R⁶ is H or CH₃; and

R⁷ and R⁸ are the same or are different and are independently selected from H and (C₁-C₄)alkyl; is preferred.

According to another embodiment, the sub-group of glucocorticoid receptor agonists of formula (Ib):

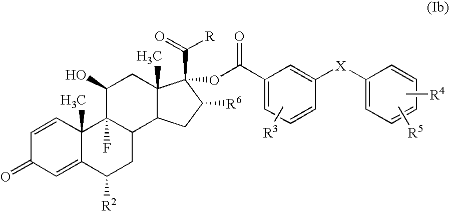

or a pharmaceutically acceptable salt thereof or a pharmaceutically acceptable solvate of said compound or salt, wherein:

### R² is H or F;

R is selected from —CH₂—OH, —O—CH₂—CN, —S—CH₂—CN, —S—CH₂F and —S—CH₂Cl;
X is a direct bond or represents a moiety selected from —O—, —S—, —CH₂—S—, —S—CH₂—, —CH₂— and —O—CH₂;

### R³ is H or OH;

### R⁴ is H or OH;

### R⁵ is H, Cl or —S—CH₃; and

### R⁶ is H or CH₃;

is further preferred.

According to another embodiment, the sub-group of glucocorticoid receptor agonists of formula (Ic):

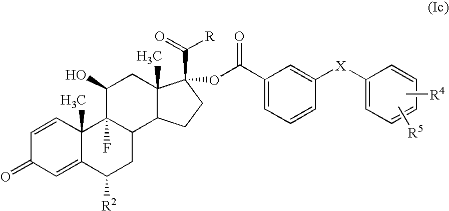

or a pharmaceutically acceptable salt thereof or a pharmaceutically acceptable solvate of said compound or salt, wherein:

### R² is H or F;

R is selected from —CH₂—OH, —O—CH₂—CN, —S—CH₂—CN, —S—CH₂F and —S—CH₂Cl;
X is a direct bond or represent a moiety selected from —O—, —S—, —CH₂—S—, —S—CH₂—, —CH₂— and —O—CH₂;

### R⁴ is H or OH; and

### R⁵ is H or Cl;

is even further preferred.

According to another embodiment, the sub-group of glucocorticoid receptor agonists of formula (Id):

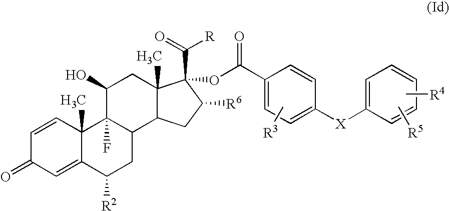

or a pharmaceutically acceptable salt thereof or a pharmaceutically acceptable solvate of said compound or salt, wherein:

### R² is H or F;

R is selected from —CH₂—OH, —O—CH₂—CN, —S—CH₂—CN, —S—CH₂F, —O—CH₂F and —S—CH₂Cl;
X is a direct bond or represents a moiety selected from —O—, —S—, —CH₂—S—, —S—CH₂—, —CH₂— and —O—CH₂;

### R³ is H or OH;

### R⁴ is H or OH;

### R⁵ is H, Cl or —S—CH₃; and

### R⁶ is H or CH₃;

is further preferred.

According to another embodiment, the sub-group of glucocorticoid receptor agonists of formula (Ie):

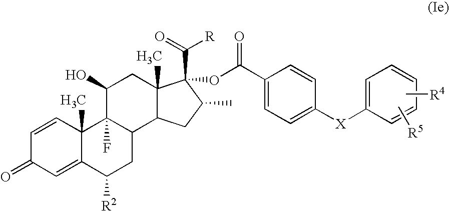

or a pharmaceutically acceptable salt thereof or a pharmaceutically acceptable solvate of said compound or salt, wherein:

### R² is H or F;

R is selected from —CH₂—OH, —O—CH₂—CN, —S—CH₂—CN, —S—CH₂F, —O—CH₂F and —S—CH₂Cl;
X is a direct bond or represents a moiety selected from —O—, —S—, —CH₂—S—, —S—CH₂—, —CH₂— and —O—CH₂;

### R⁴ is H or OH; and

### R⁵ is H or Cl;

is even further preferred.

According to yet another embodiment, the sub-group of glucocorticoid receptor agonists of formula (If):

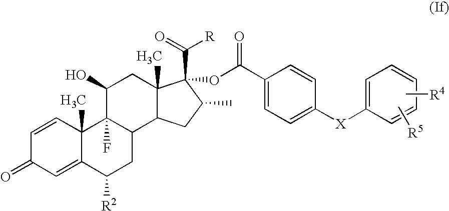

or a pharmaceutically acceptable salt thereof or a pharmaceutically acceptable solvate of said compound or salt, wherein:

### R² is H or F;

R is selected from —O—CH₂—CN, —S—CH₂—CN, —S—CH₂F and —O—CH₂F;
X is a direct bond or represents a moiety selected from —O— and —S—;

### R⁴ is OH; and

### R⁵ is Cl;

is even further preferred.

According to yet another embodiment, the sub-group of glucocorticoid receptor agonists of formula (Ig):

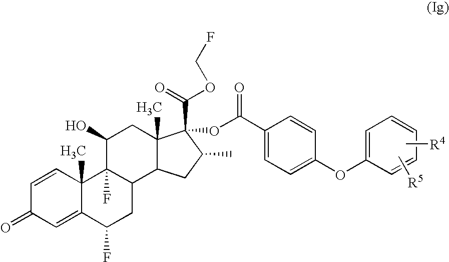

or a pharmaceutically acceptable salt thereof or a pharmaceutically acceptable solvate of said compound or salt, wherein R⁴ is OH and R⁵ is Cl;
is even further preferred.

The present invention therefore covers the following preferred compounds:
cyanomethyl(6alpha,11beta,17alpha)-17-[(4-benzylbenzoyl)oxy]-6,9-difluoro-11-hydroxy-3-oxoandrosta-1,4-diene-17-carboxylate;
cyanomethyl(6alpha,11beta,17alpha)-17-[(biphenyl-4-ylcarbonyl)oxy]-6,9-difluoro-11-hydroxy-3-oxoandrosta-1,4-diene-17-carboxylate;
cyanomethyl(6alpha,11beta,17alpha)-6,9-difluoro-11-hydroxy-3-oxo-17-{[4-(phenylthio)benzoyl]oxy}androsta-1,4-diene-17-carboxylate;
cyanomethyl(6alpha,11beta,17alpha)-6,9-difluoro-11-hydroxy-3-oxo-17-[(4-phenoxybenzoyl)oxy]androsta-1,4-diene-17-carboxylate;
cyanomethyl(6alpha,11beta,17alpha)-6,9-difluoro-11-hydroxy-3-oxo-17-({4-[(phenylthio)methyl]benzoyl}oxy)androsta-1,4-diene-17-carboxylate;
cyanomethyl(6alpha,11beta,17alpha)-6,9-difluoro-11-hydroxy-17-({4-[(4-hydroxybenzyl)thio]benzoyl}oxy)-3-oxoandrosta-1,4-diene-17-carboxylate;
cyanomethyl(11beta,17alpha)-17-[(biphenyl-4-ylcarbonyl)oxy]-9-fluoro-11-hydroxy-3-oxoandrosta-1,4-diene-17-carboxylate;
cyanomethyl(11beta,17alpha)-9-fluoro-11-hydroxy-3-oxo-17-{[4-(phenylthio)benzoyl]oxy}androsta-1,4-diene-17-carboxylate;
cyanomethyl(11beta,17alpha)-17-[(4-benzylbenzoyl)oxy]-9-fluoro-11-hydroxy-3-oxoandrosta-1,4-diene-17-carboxylate;
cyanomethyl(11beta,17alpha)-9-fluoro-11-hydroxy-17-[(4-{[3-(methylthio)-phenyl]thio}benzoyl)oxy]-3-oxoandrosta-1,4-diene-17-carboxylate;
cyanomethyl(6alpha,11beta,17alpha)-6,9-difluoro-11-hydroxy-17-[(4-{[(4-hydroxyphenyl)thio]methyl}benzoyl)oxy]-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-(3-chloro-4-hydroxyphenoxy)benzoate;
(11beta,17alpha)-17-{[(cyanomethyl)thio]carbonyl}-9-fluoro-11-hydroxy-3-oxoandrosta-1,4-dien-17-yl 4-[(3-chloro-4-hydroxyphenyl)thio]benzoate;
cyanomethyl(11beta,17alpha)-9-fluoro-11-hydroxy-3-oxo-17-[(4-phenoxybenzoyl)oxy]androsta-1,4-diene-17-carboxylate;
cyanomethyl(11beta,17alpha)-9-fluoro-11-hydroxy-17-({4-[(3-hydroxyphenyl)thio]-benzoyl}oxy)-3-oxoandrosta-1,4-diene-17-carboxylate;
cyanomethyl(11beta,17alpha)-9-fluoro-11-hydroxy-17-{[4-(4-hydroxyphenoxy)benzoyl]oxy}-3-oxoandrosta-1,4-diene-17-carboxylate;
cyanomethyl(11beta,17alpha)-9-fluoro-11-hydroxy-17-{[4-(3-hydroxyphenoxy)benzoyl]oxy}-3-oxoandrosta-1,4-diene-17-carboxylate;
cyanomethyl(11beta,17alpha)-9-fluoro-11-hydroxy-17-({4-[(4-hydroxyphenyl)thio]benzoyl}oxy)-3-oxoandrosta-1,4-diene-17-carboxylate;
cyanomethyl(11beta,17alpha)-9-fluoro-11-hydroxy-17-{[3-hydroxy-4-(phenylthio)benzoyl]oxy}-3-oxoandrosta-1,4-diene-17-carboxylate;
cyanomethyl(11beta,17alpha)-17-({4-[(3-chloro-4-hydroxyphenyl)thio]-benzoyl}oxy)-9-fluoro-11-hydroxy-3-oxoandrosta-1,4-diene-17-carboxylate;
cyanomethyl(11beta,17alpha)-9-fluoro-11-hydroxy-17-[(2-hydroxy-4-phenoxybenzoyl)oxy]-3-oxoandrosta-1,4-diene-17-carboxylate;
(11beta,17alpha)-9-fluoro-17-{[(fluoromethyl)thio]carbonyl}-11-hydroxy-3-oxoandrosta-1,4-dien-17-yl 4-benzylbenzoate;
(11beta,17alpha)-9-fluoro-17-{[(fluoromethyl)thio]carbonyl}-11-hydroxy-3-oxoandrosta-1,4-dien-17-yl 4-{[3-(methylthio)phenyl]thio}benzoate;
(11beta,17alpha)-9-fluoro-17-{[(fluoromethyl)thio]carbonyl}-11-hydroxy-3-oxoandrosta-1,4-dien-17-yl 4-(phenylthio)benzoate;
(11beta,17alpha)-9-fluoro-17-{[(fluoromethyl)thio]carbonyl}-11-hydroxy-3-oxoandrosta-1,4-dien-17-yl 4-phenoxybenzoate;
(11beta,17alpha)-9-fluoro-17-{[(fluoromethyl)thio]carbonyl}-11-hydroxy-3-oxoandrosta-1,4-dien-17-yl 4-(3-chloro-4-hydroxyphenoxy)-benzoate;
(11beta,17alpha)-9-fluoro-17-{[(fluoromethyl)thio]carbonyl}-11-hydroxy-3-oxoandrosta-1,4-dien-17-yl 4-[(3-chloro-4-hydroxyphenyl)thio]benzoate;
(11beta,17alpha)-17-{[(cyanomethyl)thio]carbonyl}-9-fluoro-11-hydroxy-3-oxoandrosta-1,4-dien-17-yl 4-(phenylthio)benzoate;
(11beta,17alpha)-17-{[(cyanomethyl)-thio]carbonyl}-9-fluoro-11-hydroxy-3-oxoandrosta-1,4-dien-17-yl 4-phenoxybenzoate;
(11beta,17alpha)-17-{[(chloromethyl)thio]carbonyl}-9-fluoro-11-hydroxy-3-oxoandrosta-1,4-dien-17-yl 4-(phenylthio)benzoate;
(6alpha,11beta)-6,9-difluoro-11,21-dihydroxy-3,20-dioxopregna-1,4-dien-17-yl 4-(benzyloxy)benzoate;
(11beta)-9-fluoro-11,21-dihydroxy-3,20-dioxopregna-1,4-dien-17-yl 4-(benzyloxy)benzoate;
cyanomethyl(11beta,17alpha)-9-fluoro-11-hydroxy-17-[(3-hydroxy-4-phenoxybenzoyl)oxy]-3-oxoandrosta-1,4-diene-17-carboxylate;
cyanomethyl(11beta,17alpha)-17-{[4-(4-chloro-3-hydroxyphenoxy)-benzoyl]oxy}-9-fluoro-11-hydroxy-oxoandrosta-1,4-diene-17-carboxylate;
cyanomethyl(11beta,17alpha)-9-fluoro-11-hydroxy-17-({4-[(2-hydroxyphenyl)thio]benzoyl}oxy)-3-oxoandrosta-1,4-diene-17-carboxylate;
cyanomethyl(11beta,17alpha)-9-fluoro-11-hydroxy-17-({4-[(6-hydroxypyridin-3-yl)oxy]benzoyl}-oxy)-3-oxoandrosta-1,4-diene-17-carboxylate;
cyanomethyl(11beta,17alpha)-17-{[4-(3-chloro-4-hydroxyphenoxy)-benzoyl]oxy}-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-[(4-chloro-3-hydroxyphenyl)thio]benzoate;
Cyanomethyl(11beta,17alpha)-17-({4-[(4-chloro-3-hydroxyphenyl)thio]benzoyl}oxy)-9-fluoro-11-hydroxy-3-oxoandrosta-1,4-diene-17-carboxylate;
(11beta,17alpha)-9-fluoro-17-{[(fluoromethyl)thio]carbonyl}-11-hydroxy-3-oxoandrosta-1,4-dien-17-yl 4-(4-chloro-3-hydroxyphenoxy)benzoate;
Fluoromethyl (6alpha,11beta,16alpha,17alpha)-17-{[4-(4-chloro-3-hydroxyphenoxy)benzoyl]oxy}-6,9-difluoro-11-hydroxy-16-methyl-3-oxoandrosta-1,4-diene-17-carboxylate;
Cyanomethyl(6alpha,11beta,16alpha,17alpha)-17-{[4-(4-chloro-3-hydroxyphenoxy)benzoyl]oxy}-6,9-difluoro-11-hydroxy-16-methyl-3-oxoandrosta-1,4-diene-17-carboxylate;
(11beta,16alpha,17alpha)-9-fluoro-17-{[(fluoromethyl)thio]carbonyl}-11-hydroxy-16-methyl-3-oxoandrosta-1,4-dien-17-yl 4-(3-chloro-4-hydroxyphenoxy)benzoate;
Fluoromethyl (6alpha,11beta,16alpha,17alpha)-17-{[4-(3-chloro-4-hydroxyphenoxy)benzoyl]oxy}-6,9-difluoro-11-hydroxy-16-methyl-3-oxoandrosta-1,4-diene-17-carboxylate;
cyanomethyl(6alpha,11beta,16alpha,17alpha)-17-{[4-(3-chloro-4-hydroxyphenoxy)benzoyl]oxy}-6,9-difluoro-11-hydroxy-16-methyl-3-oxoandrosta-1,4-diene-17-carboxylate;
Cyanomethyl(6alpha,11beta,16alpha,17alpha)-6,9-difluoro-11-hydroxy-16-methyl-17-[(4-{[4-(methylthio)phenyl]thio}benzoyl)oxy]-3-oxoandrosta-1,4-diene-17-carboxylate;
Cyanomethyl(6alpha,11beta,16alpha, 17alpha)-6,9-difluoro-11-hydroxy-16-methyl-17-({4-[3-(methylthio)phenoxy]benzoyl}oxy)-3-oxoandrosta-1,4-diene-17-carboxylate;
Cyanomethyl(6alpha,11beta,16alpha, 17alpha)-6,9-difluoro-11-hydroxy-16-methyl-17-({4-[4-(methylthio)phenoxy]benzoyl}oxy)-3-oxoandrosta-1,4-diene-17-carboxylate;
Cyanomethyl(6alpha,11beta,16alpha, 17alpha)-6,9-difluoro-11-hydroxy-16-methyl-17-[(4-{[3-(methylthio)phenyl]thio}benzoyl)oxy]-3-oxoandrosta-1,4-diene-17-carboxylate;
Fluoromethyl (6alpha, 11beta, 16alpha, 17alpha)-6,9-difluoro-11-hydroxy-16-methyl-17-[(4-{[4-(methylthio)phenyl]thio}benzoyl)oxy]-3-oxoandrosta-1,4-diene-17-carboxylate;
Fluoromethyl (6alpha, 11beta,16alpha, 17alpha)-6,9-difluoro-11-hydroxy-16-methyl-17-({4-[3-(methylthio)phenoxy]benzoyl}oxy)-3-oxoandrosta-1,4-diene-17-carboxylate;
Fluoromethyl (6alpha,11beta,16alpha, 17alpha)-6,9-difluoro-11-hydroxy-16-methyl-17-({4-[4-(methylthio)phenoxy]benzoyl}oxy)-3-oxoandrosta-1,4-diene-17-carboxylate;
Fluoromethyl (6alpha,11beta,16alpha, 17alpha)-6,9-difluoro-11-hydroxy-16-methyl-17-({4-[4-(methylthio)phenoxy]benzoyl}oxy)-3-oxoandrosta-1,4-diene-17-carboxylate;
(11beta,17alpha)-9-fluoro-17-{[(fluoromethyl)thio]carbonyl}-11-hydroxy-3-oxoandrosta-1,4-dien-17-yl 4-[(4-chloro-3-hydroxyphenyl)thio]benzoate;
(11beta,17alpha)-17-{[(cyanomethyl)thio]carbonyl}-9-fluoro-11-hydroxy-3-oxoandrosta-1,4-dien-17-yl 4-(4-chloro-3-hydroxyphenoxy)benzoate;
cyanomethyl(11beta,17alpha)-17-[(4-{[(3-chloro-4-hydroxyphenyl)thio]methyl}benzoyl)oxy]-9-fluoro-11-hydroxy-3-oxoandrosta-1,4-diene-17-carboxylate;
(11beta,17alpha)-9-fluoro-17-{[(fluoromethyl)thio]carbonyl}-11-hydroxy-3-oxoandrosta-1,4-dien-17-yl 4-{[(3-chloro-4-hydroxyphenyl)thio]methyl}benzoate;
(11beta,17alpha)-17-{[(cyanomethyl)thio]carbonyl}-9-fluoro-11-hydroxy-3-oxoandrosta-1,4-dien-17-yl 4-{[(3-chloro-4-hydroxyphenyl)thio]methyl}benzoate;
cyanomethyl(11beta,17alpha)-9-fluoro-11-hydroxy-17-[({6-[(6-hydroxypyridin-3-yl)oxy]pyridin-3-yl}carbonyl)oxy]-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 6-[(6-hydroxypyridin-3-yl)oxy]nicotinate;
(11beta,17alpha)-9-fluoro-17-{[(fluoromethyl)thio]carbonyl}-11-hydroxy-3-oxoandrosta-1,4-dien-17-yl 6-[(6-hydroxypyridin-3-yl)oxy]nicotinate;
cyanomethyl(11beta,17alpha)-9-fluoro-11-hydroxy-17-({4-[(6-hydroxypyridazin-3-yl)oxy]benzoyl}oxy)-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-[(6-hydroxypyridazin-3-yl)oxy]benzoate;
(11beta,17alpha)-9-fluoro-17-{[(fluoromethyl)thio]carbonyl}-11-hydroxy-3-oxoandrosta-1,4-dien-17-yl 4-[(6-hydroxypyridazin-3-yl)oxy]benzoate;
cyanomethyl(11beta,17alpha)-9-fluoro-11-hydroxy-17-({4-[(5-hydroxypyrazin-2-yl)oxy]benzoyl}oxy)-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-[(5-hydroxypyrazin-2-yl)oxy]benzoate;
(11beta,17alpha)-9-fluoro-17-{[(fluoromethyl)thio]carbonyl}-11-hydroxy-3-oxoandrosta-1,4-dien-17-yl 4-[(5-hydroxypyrazin-2-yl)oxy]benzoate;
cyanomethyl(11beta,17alpha)-17-{[4-(3-cyano-4-hydroxyphenoxy)benzoyl]oxy}-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-(3-cyano-4-hydroxyphenoxy)benzoate;
(11beta,17alpha)-9-fluoro-17-{[(fluoromethyl)thio]carbonyl}-11-hydroxy-3-oxoandrosta-1,4-dien-17-yl 4-(3-cyano-4-hydroxyphenoxy)benzoate;
cyanomethyl(11beta,17alpha)-17-{[4-(4-cyano-3-hydroxyphenoxy)benzoyl]oxy}-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-(4-cyano-3-hydroxyphenoxy)benzoate;
(11beta,17alpha)-9-fluoro-17-{[(fluoromethyl)thio]carbonyl}-11-hydroxy-3-oxoandrosta-1,4-dien-17-yl 4-(4-cyano-3-hydroxyphenoxy)benzoate;
cyanomethyl(11beta,17alpha)-17-{[4-(3-carbamoyl-4-hydroxyphenoxy)benzoyl]oxy}-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-(3-carbamoyl-4-hydroxyphenoxy)benzoate;
(11beta,17alpha)-9-fluoro-17-{[(fluoromethyl)thio]carbonyl}-11-hydroxy-3-oxoandrosta-1,4-dien-17-yl 4-(3-carbamoyl-4-hydroxyphenoxy)benzoate;
cyanomethyl(11beta,17alpha)-17-{[4-(4-carbamoyl-3-hydroxyphenoxy)benzoyl]oxy}-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-(4-carbamoyl-3-hydroxyphenoxy)benzoate;
(11beta,17alpha)-9-fluoro-17-{[(fluoromethyl)thio]carbonyl}-11-hydroxy-3-oxoandrosta-1,4-dien-17-yl 4-(4-carbamoyl-3-hydroxyphenoxy)benzoate;
cyanomethyl(11beta,17alpha)-17-({4-[3-(dimethylcarbamoyl)-4-hydroxyphenoxy]benzoyl}oxy)-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-[3-(dimethylcarbamoyl)-4-hydroxyphenoxy]benzoate;
(11beta,17alpha)-9-fluoro-17-{[(fluoromethyl)thio]carbonyl}-11-hydroxy-3-oxoandrosta-1,4-dien-17-yl 4-[3-(dimethylcarbamoyl)-4-hydroxyphenoxy]benzoate;
cyanomethyl(11beta,17alpha)-17-({4-[4-(dimethylcarbamoyl)-3-hydroxyphenoxy]benzoyl}oxy)-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-[4-(dimethylcarbamoyl)-3-hydroxyphenoxy]benzoate; and
(11beta,17alpha)-9-fluoro-17-{[(fluoromethyl)thio]carbonyl}-11-hydroxy-3-oxoandrosta-1,4-dien-17-yl 4-[4-(dimethylcarbamoyl)-3-hydroxyphenoxy]benzoate.

More preferred glucocorticoid receptor agonist according to the present invention are:
Cyanomethyl(11beta,17alpha)-17-{[4-(4-chloro-3-hydroxyphenoxy)-benzoyl]oxy}-9-fluoro-11-hydroxy-oxoandrosta-1,4-diene-17-carboxylate;
Fluoromethyl (6alpha,11beta,16alpha,17alpha)-17-{[4-(3-chloro-4-hydroxyphenoxy)benzoyl]oxy}-6,9-difluoro-11-hydroxy-16-methyl-3-oxoandrosta-1,4-diene-17-carboxylate; and
Fluoromethyl (6alpha,11beta,16alpha,17alpha)-17-{[4-(4-chloro-3-hydroxyphenoxy)benzoyl]oxy}-6,9-difluoro-11-hydroxy-16-methyl-3-oxoandrosta-1,4-diene-17-carboxylate.

Most preferred glucocorticoid receptor agonist according to the present invention are fluoromethyl (6alpha,11beta,16alpha,17alpha)-17-{[4-(3-chloro-4-hydroxyphenoxy)benzoyl]oxy}-6,9-difluoro-11-hydroxy-16-methyl-3-oxoandrosta-1,4-diene-17-carboxylate and fluoromethyl (6alpha,11beta,16alpha,17alpha)-17-{[4-(4-chloro-3-hydroxyphenoxy)benzoyl]oxy}-6,9-difluoro-11-hydroxy-16-methyl-3-oxoandrosta-1,4-diene-17-carboxylate.

The compounds of formula (I) according to the present invention may be prepared in a variety of ways using conventional procedures such as by the following illustrative methods in which R¹, R², R³, R⁴, R⁵, R⁶, R⁷, R⁸, R, X, Ar¹ and Ar² are as previously defined for the compounds of the formula (I) unless otherwise stated. But the skilled person will appreciate that other routes may be equally as practicable.

The compounds of formula (I) may be prepared according to Scheme 1 or Scheme 3 as follows:

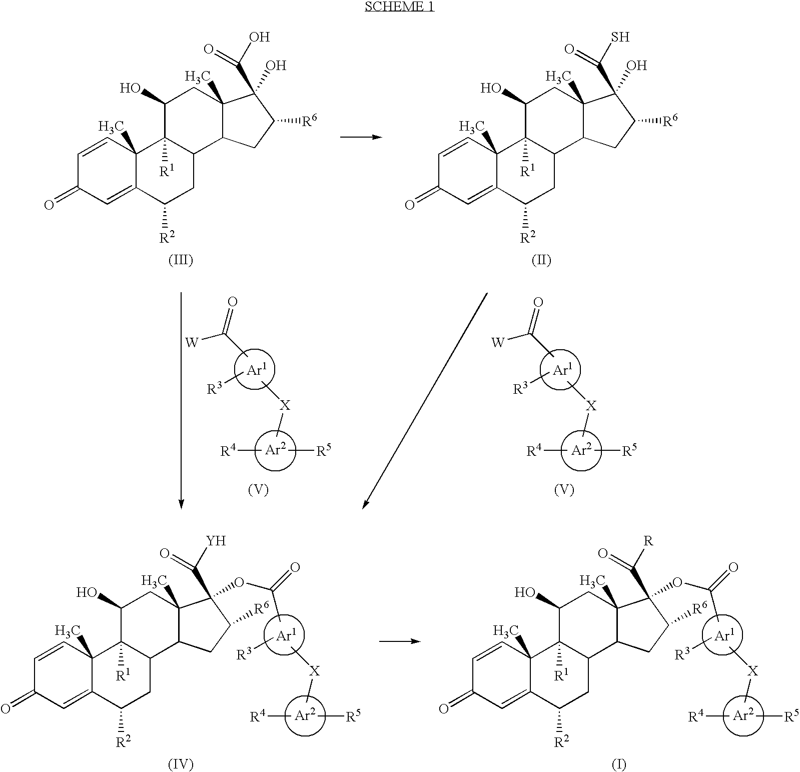

wherein Y is O or S and W is chloro or O-(7-Azabenzotriazol-1-yl).

According to Scheme 1, compounds of formula (IV) may be prepared by the reaction of a compound of formula (II) or (III) with a suitable activated carboxylic acid of formula (V). This is typically a carboxylic acid chloride or activated carboxylic ester (preferably O-(7-Azabenzotriazol-1-yl)).

Conveniently reaction of (II) or (III) to (IV) is effected by using an excess of the activated carboxylic acid of formula (V), or stoichiometric quantity of the activated carboxylic acid of formula (V), in the presence of a base such as triethylamine, N,N-diisopropylethylamine or pyridine and in the presence of a suitable solvent (e.g. acetone, N,N-dimethylformamide or dichloromethane), and at ambient temperature.

Finally, compounds of formula (I) for which R is —O—CH₂—CN, —S—CH₂—CN, —O—CH₂F or —S—CH₂F are prepared by reaction of compounds of formula (IV) with a suitable alkylating agent such as bromoacetonitrile, in the presence of sodium hydrogen carbonate and in the presence of a suitable solvent, such as N,N-dimethyl formamide, at ice temperature or at ambient temperature. Alternatively, compounds of formula (I) may be prepared from compounds of formula (IV) by reaction with suitable alkylating agent such as bromofluoromethane or bromochloromethane, either as a gas bubbled through the reaction mixture, or as a solution in 2-butanone, in the presence of N,N-diisopropylethylamine and in the presence of a suitable solvent, such as acetonitrile, at ice temperature or at ambient temperature.

The acid chlorides of formula (V) are typically prepared from the corresponding carboxylic acid precursors by treatment with oxalyl chloride in dichloromethane in the presence of a catalytic amount of dimethylformamide followed by concentration in vacuo and are typically used without purification. The activated carboxylic esters of formula (V) are typically prepared from the corresponding carboxylic acid precursors by treatment with N,N-diisopropylethylamine and o-(7-azabenzotriazol-1-yl)-N,N,N′,N′ tetramethyluronium hexafluorophosphate in dimethylformamide and used without isolation or purification.

the carboxylic acid precursors are commercially available or alternatively, when not commercially available, the carboxylic acid precursors are typically prepared as below:

### When X is a Direct Bond:

The carboxylic acid precursor can be prepared from a suitably substituted phenol or thiophenol of formula Ar²—OH or Ar²—SH, wherein Ar² is as defined in formula (I), and a substituted 4-fluorobenzonitrile of formula NC—Ar¹—F, wherein Ar¹ is as defined in formula (I), in the presence of a suitable base such as cesium carbonate, additives such as 2-hydroxybenzaldehyde oxime and copper (I) oxide, and a suitable solvent such as N,N-dimethylformamide or acetonitrile. The substituted benzonitriles thus obtained can then be hydrolysed to the carboxylic acid by means of a strong base, typically sodium or potassium hydroxide, in a suitable solvent, typically aqueous ethanol or methanol.

Alternatively the carboxylic acid precursor can be prepared from a suitably substituted phenol or thiophenol of formula Ar²—OH or Ar²—SH and a substituted 4-fluorobenzaldehyde of formula OHC—Ar¹—F in the presence of a suitable base such as cesium carbonate and a suitable solvent such as N,N-dimethylformamide or acetonitrile. The substituted benzaldehydes thus obtained can then be oxidised to the carboxylic acid by tert-butyl hydroperoxide and copper(I) chloride in a suitable solvent, typically acetonitrile.

Alternatively the carboxylic acid precursor can be prepared from a suitably substituted phenol or thiophenol of formula Ar²—OH or Ar²—SH and a substituted 4-iodobenzonitrile of formula NC—Ar¹—I in the presence of tripotassium phosphate, copper (I) iodide and N,N,N-tributylbutan-1-aminium bromide in a suitable solvent such as N,N-dimethylformamide.

Alternatively the carboxylic acid precursor can be prepared from a suitably substituted phenol or thiophenol of formula Ar²—OH or Ar²—SH and a 4-substituted aryl boronic acid of formula MeO₂C—Ar¹—B(OH)₂ in the presence of copper (I) iodide and 2,2′-bipyridine in a suitable solvent such as dimethylsulphoxide. The substituted methyl benzoates thus obtained can then be hydrolysed to the carboxylic acid by treatment with lithium hydroxide in a suitable solvent such as THF/water or dioxane/water.

### When X is a Contains a CH₂Group:

The carboxylic acid precursor can be prepared by reaction of a suitably substituted phenol or thiophenol of formula Ar²—OH or Ar²—SH, or a suitably substituted phenol or thiophenol of formula Ar¹—OH or Ar¹—SH with a suitably substituted benzyl bromide in the presence of a suitable base such as cesium carbonate or triethylamine and a suitable solvent such as dioxane of N,N-dimethylformamide.

When not commercially available the substituted thiophenols of formula Ar²—OH and Ar²—SH can be prepared from a suitably substituted phenyl compound by treatment with sodium thiocyanate in a suitable solvent such as acetic acid. The thiocyanates thus obtained can be reduced to the thiophenols by treatment with a suitable reducing agent, such as lithium aluminium hydride, in a suitable solvent such as THF.

The compounds (III) can all be prepared from compounds (VI) (Scheme 2) by means of an oxidative cleavage reaction. Typically compounds (VI) are treated with potassium carbonate in methanol at ambient temperature and air is bubbled through the reaction mixture for 2 hours. After acidic work-up the compounds are isolated by filtration and are typically used without further purification.

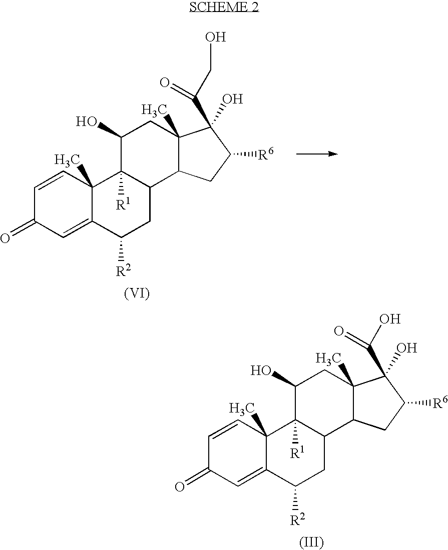

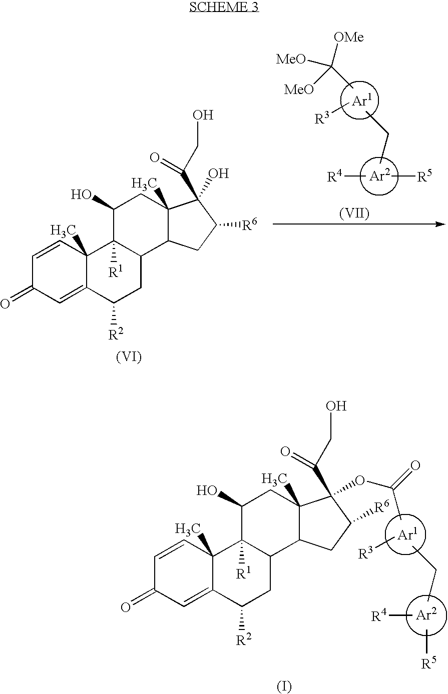

According to scheme 3, the compounds of formula (I) wherein R is —CH₂—OH may be prepared by reaction of a compound of formula (VI) with an excess of an arylorthoester of formula (VII), typically in the presence of an acid such as para-toluene sulfonic acid, in a suitable solvent such as toluene or 1,4-dioxane at elevated temperature.

The arylorthoester of formula (VII) is either commercially available or, when not commercially available, the aryl orthoesters of formula (VII) may be prepared by reaction of a suitable orthoester phenol such as 4-(trimethoxymethyl)phenol with a suitably substituted benzyl bromide in the presence of a suitable base such as cesium carbonate and a suitable solvent such as N,N-dimethylformamide.

Alternatively the aryl orthoesters of formula (VII) may be prepared by reaction of a suitable orthoester bromide such as 1-bromo-4-(trimethoxymethyl)benzene and a suitable phenol or thiophenol of formula Ar²—OH or Ar²—SH in the presence of tripotassium phosphate, copper (I) iodide and N,N,N-tributylbutan-1-aminium bromide in a suitable solvent such as N,N-dimethylformamide

The compounds of formula (V) are either commercially available or they may be easily prepared as taught in the chemical literature (see e.g. JOC 1961 p 2863-2867, JACS 1958 p 6464-6465, JOC 1961 p 2426-2431. FR1215564, U.S. Pat. No. 3,053,832, GB926472, Chemistry & Industry (London, United Kingdom) (1960), p. 1163-4 and U.S. Pat. No. 3,049,556).

For some of the steps of the hereinbefore described process of preparation of the compounds of formula (I), it may be necessary to protect potential reactive functions that are not wished to react, and subsequently to cleave said protecting groups. In such a case, any compatible protecting radical can be used. In particular methods of protection and deprotection such as those described by T. W. GREENE (Protective Groups in Organic Synthesis, A. Wiley-Interscience Publication, 1981) or by P. J. Kocienski (Protecting groups, Georg Thieme Verlag, 1994), can be used.

All of the above reactions and the preparations of novel starting materials used in the preceding methods are conventional and appropriate reagents and reaction conditions for their performance or preparation as well as procedures for isolating the desired products will be well-known to those skilled in the art with reference to literature precedents and the examples and preparations herein.

Also, the compounds of formula (I) as well as intermediate for the preparation thereof can be purified according to various well-known methods, such as for example crystallization or chromatography.

Pharmaceutically acceptable salts of the compounds of formula (I) include the base salts thereof. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Pharmaceutically acceptable salts of the compounds of formula (I) may also eventually include the acid salts thereof. Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention and salts thereof may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to the compounds of the invention include references to salts, solvates, multi-component complexes and liquid crystals thereof and to solvates, multi-component complexes and liquid crystals of salts thereof.

The compounds of the invention include compounds of formula (I) as hereinbefore defined, including all polymorphs and crystal habits thereof, prodrugs and isomers thereof (including optical, geometric and tautomeric isomers) as hereinafter defined and isotopically-labeled compounds of formula (I).

As indicated, so-called ‘prodrugs’ of the compounds of the invention are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Some examples of prodrugs in accordance with the invention include, where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl.

Further examples of replacement groups in accordance with the foregoing examples and examples of other prodrug types may be found in the aforementioned references.

Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include:
(i) where the compound of formula (I) contains a methyl group, an hydroxymethyl derivative thereof (—CH₃→—CH₂OH);
(ii) where the compound of formula (I) contains a phenyl moiety, a phenol derivative thereof (-Ph→-PhOH); and
(iii) where the compound of formula (I) contains a sulfide, a sulfoxide derivative thereof (—SPh→—S(O)Ph).

Compounds of formula (I) containing one or more asymmetric carbon atoms can exist as two or more stereoisomers. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism. Included within the scope of the present invention are all stereoisomers, geometric isomers and tautomeric forms of the compounds of formula I, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

The compounds of formula (I) should be assessed for their biopharmaceutical properties, such as solubility and solution stability (across pH), permeability, etc., in order to select the most appropriate dosage form and route of administration for treatment of the proposed indication.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention such as for example diluents, carriers and adjuvants. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

The compounds of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 0.001 mg to 10 mg of the compound of formula (I). The overall daily dose will typically be in the range 0.001 mg to 40 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of formula (I) according to the present invention are particularly suitable for nasal, inhaled and topical administration.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range 0.001 mg to 5000 mg, preferably in the range of 0.01 mg to 1000 mg, depending, of course, on the mode of administration. For example, oral administration or intravenous, intramuscular, intra-articular or peri-articular administration may require a total daily dose of from 0.01 mg to 1000 mg, preferably from 0.01 mg to 100 mg. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

The compounds of formula (I) have the ability to interact with glucocorticoid receptor and thereby have a wide range of therapeutic applications, as described further below, because of the essential role which the glucocortocoid receptor plays in the physiology of all mammals.

Thus the invention relates to the compounds of formula (I), or pharmaceutically acceptable salts thereof or pharmaceutically acceptable solvates of said compounds or salts, for use in the treatment or the prevention of diseases, disorders, and conditions in which the glucocorticoid receptor is involved. The invention further relates to the use of the compounds of formula (I), or pharmaceutically acceptable salts thereof or pharmaceutically acceptable solvates of said compounds or salts, for the manufacture of a medicament for the treatment of diseases, disorders, and conditions in which the glucocorticoid receptor is involved. The invention also further relates to a method of treatment of a mammal, including a human being, with a glucocorticoid receptor agonist including treating said mammal with an effective amount of a compound of the formula (I) or with a pharmaceutically acceptable salt thereof or a pharmaceutically acceptable solvate of said compound or salt.

Examples of such diseases, disorders, and conditions include skin diseases such as eczema, psoriasis, dermatitis, pruritis and hypersensitivity reactions; inflammatory conditions of the nose, throat and lungs such as rhinitis, sinusitis, asthma, nasal polyps, chronic obstructive pulmonary disease (COPD) and fibrosis; inflammatory diseases of the intestine such as inflammatory bowel disease, Crohn's disease and ulcerative colitis; auto-immune diseases such as rheumatoid arthritis; multiple sclerosis and disseminated lupus erythematosus; ocular conditions, such as non-infected inflammation (conjunctivitis). The compounds may also have application in cancer (e.g. gliomas and prostate cancer), acquired immuno-deficiency syndrome, osteoarthritis, septic shock, graft rejection, emphysema (especially by patients having COPD), post-ischaemic lesions, pulmonary hypertension, acute respiratory distress syndrome, prevention of restenosis after coronary angioplasty, Stevens-Johnson syndrome, HELLP syndrome (a variant form of severe pre-eclampsia), pneumonia, chronic active hepatitis, haematological disorders, renal disease, and acute spinal cord injury.

Preferably, the compounds according to the present invention are used for the treatment of:
skin diseases such as eczema, psoriasis, dermatitis, pruritis and hypersensitivity reactions;
inflammatory conditions of the nose, throat and lungs such as rhinitis, sinusitis, asthma, nasal polyps, chronic obstructive pulmonary disease (COPD) and fibrosis;
inflammatory diseases of the intestine such as inflammatory bowel disease, Crohn's disease and ulcerative colitis;
auto-immune diseases such as rheumatoid arthritis; and
ocular conditions, such as conjunctivitis.

The skin diseases that are treated by the compounds of the present invention may be of whatever type, etiology, or pathogenesis, in particular eczema, psoriasis, allergic dermatitis, neurodermatitis. pruritis and hypersensitivity reactions.

Rhinitis that is treated by the compounds of the present invention may be seasonal allergic rhinitis or perennial allergic rhinitis.

Sinusitis that is treated by the compounds of the present invention may be of whatever type, etiology, or pathogenesis, in particular sinusitis that is a member selected from the group consisting of purulent or nonpurulent sinusitis, acute or chronic sinusitis and ethmoid, frontal, maxillary, or sphenoid sinusitis.

Asthma that is treated by the compounds of the present invention may be of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis.

Obstructive or inflammatory airways diseases that are treated by the compounds of the present invention may be of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension.

Fibrosis that is treated by the compounds of the present invention may be of whatever type, etiology, or pathogenesis, in particular pulmonary fibrosis associated with inflammatory airway disease.

Inflammatory diseases of the intestine that are treated by the compounds of the present invention may be of whatever type, etiology, or pathogenesis, in particular ulcerative colitis and Crohn's disease.

Finally, the auto-immune diseases that are treated by the compounds of the present invention may be of whatever type, etiology, or pathogenesis, in particular rheumatoid arthritis, multiple sclerosis, and disseminated lupus erythematosus,

Even more specifically, the compounds according to the present invention are more specifically useful for the treatment of asthma, COPD, allergic rhinitis, nasal polyps, Crohn's disease, eczema, and psoriasis.

According to another embodiment of the present invention, the compounds of the invention, or pharmaceutically acceptable salts thereof or pharmaceutically acceptable solvates of said compounds or salts, can also be used as a combination with one or more additional therapeutic agents to be co-administered to a patient to obtain some particularly desired therapeutic end result such as the treatment of pathophysiologically-relevant disease processes including, but not limited to (i) bronchoconstriction, (ii) inflammation, (iii) allergy, (iv) tissue destruction, (v) signs and symptoms such as breathlessness, cough. The second and more additional therapeutic agents may also be a compound of the formula (I), or a pharmaceutically acceptable salt thereof or a pharmaceutically acceptable solvate of said compound or salt, or one or more glucocorticoid receptor agonists known in the art. More typically, the second and more therapeutic agents will be selected from a different class of therapeutic agents.

As used herein, the terms “co-administration”, “co-administered” and “in combination with”, referring to the compounds of the invention and one or more other therapeutic agents, is intended to mean, and does refer to and include the following:
simultaneous administration of such combination of compound(s) of formula (I) and therapeutic agent(s) to a patient in need of treatment, when such components are formulated together into a single dosage form which releases said components at substantially the same time to said patient;
substantially simultaneous administration of such combination of compound(s) of formula (I) and therapeutic agent(s) to a patient in need of treatment, when such components are formulated apart from each other into separate dosage forms which are taken at substantially the same time by said patient, whereupon said components are released at substantially the same time to said patient;
sequential administration of such combination compound(s) of formula (I) and therapeutic agent(s) to a patient in need of treatment, when such components are formulated apart from each other into separate dosage forms which are taken at consecutive times by said patient with a significant time interval between each administration, whereupon said components are released at substantially different times to said patient; and
sequential administration of such combination of compound(s) of formula (I) and therapeutic agent(s) to a patient in need of treatment, when such components are formulated together into a single dosage form which releases said components in a controlled manner whereupon they are concurrently, consecutively, and/or overlapping administered at the same and/or different times by said patient;
   where each part may be administered by either the same or different route.

Suitable examples of other therapeutic agents which may be used in combination with the compounds of the invention, or pharmaceutically acceptable salts thereof or pharmaceutically acceptable solvates of said compounds or salts, include, but are by no means limited to:
(a) 5-Lipoxygenase (5-LO) inhibitors or 5-lipoxygenase activating protein (FLAP) antagonists;
(b) Leukotriene antagonists (LTRAs) including antagonists of LTB₄, LTC₄, LTD₄, and LTE₄;
(c) Inhibitors of leukotriene C4 synthase;
(d) Histamine receptor antagonists including H1, H3 and H4 antagonists;
(e) α₁- and α₂-adrenoceptor agonist vasoconstrictor sympathomimetic agents for decongestant use;
(f) PDE inhibitors, including PDE3, PDE4 and PDE5 inhibitors;
(g) Theophylline;
(h) Sodium cromoglycate;
(i) COX inhibitors selected from both non-selective and selective COX-1 or COX-2 inhibitors (NSAIDs);
(j) Prostaglandin receptor antagonists and inhibitors of prostaglandin synthase, including hPGDS;
(k) Muscarinic M3 receptor antagonists or anticholinergic agents;
(l) β2-adrenoceptor agonists;
(m) Monoclonal antibodies active against endogenous proinflammatory entities, including IgE, IL3, IL4, IL9, IL10, IL13, IL17A, GMCSF and their receptors;
(n) Anti-tumor necrosis factor (anti-TNF-α) agents;
(o) Adhesion molecule inhibitors including VLA-4 antagonists;
(p) Kinin-B₁- and B₂-receptor antagonists;
(q) Immunosuppressive agents, including inhibitors of the IgE pathway and cyclosporine;
(r) Inhibitors of matrix metalloproteases (MMPs) including MMP9 and MMP12;
(s) Tachykinin NK₁, NK₂ and NK₃ receptor antagonists;
(t) Protease inhibitors such as elastase inhibitors including neutrophil elastase inhibitors;
(u) Adenosine A2a receptor agonists and A2b antagonists;
(v) Inhibitors of urokinase;
(w) Compounds that act on dopamine receptors including D2 agonists;
(x) Modulators of the NFκβ pathway including IKK inhibitors;
(y) modulators of cytokine signalling pathyways including p38 MAP kinase, PI3 kinases, JAK kinases, syk kinase, EGFR, MK-2, fyn kinases or ITK;
(z) Agents that can be classed as mucolytics or anti-tussive;
(aa) Agents, which enhance or re-sensitise responses to inhaled corticosteroids such as macolide analogues and inhibitors of PI3Kδ or AKT1,2,3;
(bb) Antibiotics and antiviral agents effective against micro-organisms which can colonise the respiratory tract;
(cc) HDAC activators;
(dd) CXCR1, CXCR2 and CXCR3 antagonists;
(ee) Integrin antagonists;
(ff) Chemokines and chemokine receptor antagonists;
(gg) Epithelial sodium channel (ENaC) blockers or Epithelial sodium channel (ENaC) inhibitors;
(hh) CRAC ion channel blockers or CRAC inhibitors;
(ii) P2Y2 Agonists and other Nucleotide receptor agonists;
(jj) P2X7 antagonists;
(kk) Inhibitors of VAP1;
(ll) Inhibitors of thromboxane;
(mm) Niacin; and
(nn) Adhesion factors including VLAM, ICAM, and ELAM.

According to the present invention, the combinations of the compounds of formula (I), or pharmaceutically acceptable salts thereof or pharmaceutically acceptable solvates of said compounds or salts, with:
muscarinic M3 receptor agonists or anticholinergic agents such as ipratropium salts, namely bromide, tiotropium salts, namely bromide, oxitropium salts, namely bromide, trospium salts, aclidinium salts, perenzepine, and telenzepine;
β2-adrenoceptor agonists such as ephedrine, adrenaline, isoprenaline, metaproterenol, phenylephrine, phenylpropanolamine, pirbuterol, reproterol, rimiterol, isoetharine, tolobuterol, carmoterol, albuterol, terbutaline, bambuterol, fenoterol, salbutamol, tulobuterol formoterol, salmeterol, as well as salts thereof and the agonists described in WO 05/080313, WO 05/080324 and WO 05/092840;
PDE4 inhibitors, in particular inhaled PDE4 inhibitors;
Theophylline;
Histaminic receptor antagonists including H1 and H3 antagonists such as loratadine and methapyrilene; or
adenosine A2a receptor agonists such as the agonists described in WO01/94368; are preferred.

According to a preferred aspect, the compounds of the present invention may be combined with another therapeutic agent selected from β2-adrenoceptor agonists and anticholinergic agents. Another preferred aspect includes the triple combination of a compound according to the present invention together with a β2-adrenoceptor agonist and an anticholinergic agent.

The following non-limiting examples illustrate the invention:

### EXAMPLES

### Preparation 1

### (6alpha,11beta,17alpha)-6,9-difluoro-11,17-dihydroxy-3-oxoandrosta-1,4-diene-17-carboxylic acid

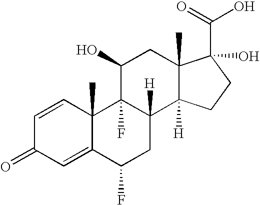

A suspension of (6alpha,11beta)-6,9-difluoro-11,17,21-trihydroxypregna-1,4-diene-3,20-dione (4.98 g, 12.60 mmol) in methanol (290 mL) was treated with potassium carbonate (3.89 g, 28.10 mmol). Air was bubbled through the resulting suspension for 2 hours. After stirring at ambient temperature for 18 hours the reaction mixture was concentrated in vacuo and the residue was dissolved in water (100 mL). The resulting solution was extracted with ethyl acetate (3×75 mL) and then acidified by the addition of hydrochloric acid (2N aqueous solution) to a pH of approximately 4.5 leading to the precipitation of the title compound which was filtered off as a pale yellow solid, 2.88 g, 60% yield.

The filtrate was extracted with ethyl acetate (4×50 mL) and the combined organic extracts were dried (magnesium sulphate) and concentrated in vacuo to give further crop of the title compound as a yellow solid, 1.67 g, 35% yield.

¹H NMR (400 MHz, DMSO-d₆) δ: 0.92 (s, 3H), 1.27-1.39 (m, 1H), 1.52 (s, 3H), 1.45-1.68 (m, 4H), 1.98-2.09 (m, 2H), 2.26-2.32 (m, 1H), 2.43-2.58 (m, 2H), 4.14-4.19) (m, 1H), 4.97 (br s, 1H), 5.33-5.34) (m, 1H), 5.58-5.75 (m, 1H), 6.12 (m, 1H), 6.30 (dd, 1H), 7.28 (m, 1H), 12.30 (br s, 1H) ppm.

LRMS (ESI): m/z 383 [M+H]⁺381 [M−H]⁻

### Preparation 2

### (6alpha,11beta, 17alpha)-17-[(4-benzylbenzoyl)oxy]-6,9-difluoro-11-hydroxy-3-oxoandrosta-1,4-diene-17-carboxylic acid

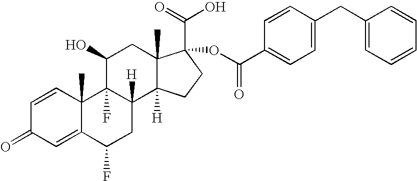

4-Benzylbenzoyl chloride was prepared from 4-benzylbenzoic acid following the method of Preparation 5 by treatment with oxalyl chloride in dichloromethane in the presence of a catalytic amount of dimethylformamide followed by concentration in vacuo and used without isolation or purification. A suspension of (6alpha, 11beta, 17alpha)-6,9-difluoro-11,17-dihydroxy-3-oxoandrosta-1,4-diene-17-carboxylic acid as obtained in Preparation 1 (205 mg, 0.54 mmol) in dichloromethane (9 mL) was cooled to 0° C. and treated with triethylamine (150 μL, 1.08 mmol). A solution of 4-benzylbenzoyl chloride (240 mg, 1.04 mmol) in dichloromethane (2 mL) was added dropwise at 0° C. and the reaction mixture was then allowed to warm to ambient temperature. After 18 hours stirring at ambient temperature the reaction mixture was diluted with dichloromethane (10 mL) and washed with saturated sodium hydrogen carbonate solution (5 mL, aqueous), water (5 mL), dried (magnesium sulphate) and concentrated in vacuo. The resulting yellow solid was dissolved in acetone (10 mL) and treated with diethylamine (277 μL, 2.68 mmol) and stirred at ambient temperature. After 42 hours stirring at ambient temperature the suspension was concentrated in vacuo and the residue was dissolved in water (10 mL) and washed with ethyl acetate (10 mL). The aqueous phase was acidified to pH 2 by the addition of hydrochloric acid (2N aqueous solution) and extracted with ethyl acetate (20 mL). The organic phases were combined, concentrated in vacuo and purified by flash column chromatography on silica gel eluting with ethyl acetate:methanol:acetic acid (1:0:0 changing to 40:9:1, by volume) to give the title compound as a yellow foam, 98 mg, 32% yield.

¹H NMR (400 MHz, DMSO-d₆) δ: 1.04 (s, 3H), 1.54 (s, 3H), 1.42-1.63 (m, 2H), 1.66-1.78 (m, 2H), 1.88-1.98 (m, 1H), 2.15-2.36 (m, 3H), 2.56-2.72 (m, 1H), 2.81-2.89 (m, 1H), 4.01 (s, 2H), 4.27-4.32 (m, 1H), 5.52-5.53 (m, 1H), 5.59-5.78 (m, 1H), 6.14 (m, 1H), 6.34 (dd, 1H), 7.17-7.24 (m, 3H), 7.27-7.33 (m, 3H), 7.40-7.42 (m, 2H), 7.77-7.80 (m, 2H) ppm.

LRMS (API): m/z 577 [M+H]⁺ 575 [M−H]⁻

LRMS (ESI): m/z 577 [M+H]⁺ 575 [M−H]⁻

### Preparation 3

### (11beta,17alpha)-9-fluoro-11,17-dihydroxy-3-oxoandrosta-1,4-diene-17-carbothioic S-acid

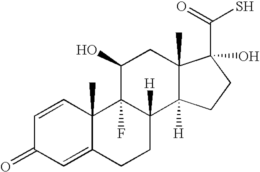

A solution of (11beta,17alpha)-9-fluoro-11,17-dihydroxy-3-oxoandrosta-1,4-diene-17-carboxylic acid [Phillipps et al, Journal of Medicinal Chemistry, 1994, pages 3717-3729] (1.20 g, 3.30 mmol) in dimethylformamide (30 mL) was treated with 1,1′-carbonyl diimidazole (1.07 g, 6.60 mmol). The resulting solution was stirred at ambient temperature for 1.5 hours before hydrogen sulphide was bubbled through the solution for 5 minutes. After stirring at ambient temperature for 10 minutes the solution was poured into hydrochloric acid (2N aqueous solution, 50 mL) and then diluted with water (30 mL). The resulting suspension was filtered to collect a white solid which was suspended in methanol (50 mL) and concentrated in vacuo to give the title compound as a white solid, 1.20 g, 96% yield.

¹H NMR (400 MHz, DMSO-d₆) δ: 0.89 (s, 3H), 1.29-1.45 (m, 2H), 1.52 (s, 3H), 1.50-1.68 (m, 3H), 1.81-1.87 (m, 1H), 2.00-2.13 (m, 2H), 2.33-2.49 (m, 3H), 2.61-2.70 (m, 1H), 3.19 (s, 1H), 4.15-4.21 (m, 1H), 5.30 (m, 1H), 6.03 (m, 1H), 6.24 (dd, 1H), 7.31 (d, 1H) ppm.

LRMS (ESI): m/z 381 [M+H]⁺

### Preparation 4

### (11beta,17alpha)-17-[(4-benzylbenzoyl)oxy]-9-fluoro-11-hydroxy-3-oxoandrosta-1,4-diene-17-carbothioic S-acid

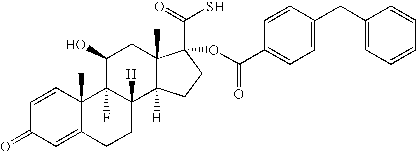

4-benzylbenzoyl chloride was prepared from 4-benzylbenzoic acid following the method of Preparation 5 by treatment with oxalyl chloride in dichloromethane in the presence of a catalytic amount of dimethylformamide followed by concentration in vacuo and used without isolation or purification. A suspension of (11beta,17alpha)-9-fluoro-11,17-dihydroxy-3-oxoandrosta-1,4-diene-17-carbothioic S-acid as obtained in Preparation 3 (313 mg, 0.82 mmol) in dichloromethane (25 mL) was cooled to 0° C. and treated with triethylamine (288 μL, 2.06 mmol). The resulting solution was stirred at 0° C. and treated with a solution of 4-benzylbenzoyl chloride (418 mg, 1.81 mmol) in dichloromethane (10 mL, over 2 minutes). The resulting solution was allowed to warm to ambient temperature. After stirring for 18 hours the solution was diluted with dichloromethane (20 mL) and washed with saturated sodium hydrogen carbonate solution (30 mL, aqueous), brine (30 mL) and dried (sodium sulphate) and concentrated in vacuo. The resulting yellow solid was dissolved in acetone (10 mL) and treated with diethylamine (341 μL, 0.33 mmol). After stirring at ambient temperature for 18 hours the resulting solution was suspended in hydrochloric acid (1N aqueous solution, 30 mL) and extracted with ethyl acetate (3×30 mL). The combined organic extracts were washed with brine (30 mL), dried (sodium sulphate) and concentrated in vacuo. The residue was purified by flash column chromatography on silica gel eluting with heptane:ethyl acetate:methanol:acetic acid (1:0:0:0 to 0:55:14:1, by volume, gradient elution) to give the title compound as a white solid, 304 mg, 54% yield.

¹H NMR (400 MHz, DMSO-d₆) δ: 1.02 (s, 3H), 1.36-1.47 (m, 2H), 1.54 (s, 3H), 1.61-1.71 (m, 1H), 1.83-2.14 (m, 4H), 2.31-2.52 (m, 3H), 2.62-2.71 (m, 1H), 2.88-2.97 (m, 1H), 4.01 (s, 2H), 4.29-4.34 (m, 1H), 5.47-5.48 (m, 1H), 6.05 (m, 1H), 6.27-6.30 (m, 1H), 7.18-7.35 (m, 6H), 7.42-7.44 (m, 2H), 7.80-7.83 (m, 2H) ppm.

LRMS (ESI): m/z 575 [M+H]⁺

### Preparation 5

### 4-[(phenylthio)methyl]benzoyl chloride

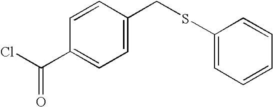

A solution of 4-[(phenylthio)methyl]benzoic acid [DeGraw, Journal of Medicinal Chemistry, 1984, 376-380] (159 mg, 0.65 mmol) in dichloromethane (2.2 mL) was treated with dimethylformamide (100 μL) and cooled to 0° C. The resulting solution was treated with oxalyl chloride (62 μL, 0.72 mmol) and allowed to warm to ambient temperature. After stirring for 3 hours the solution was concentrated in vacuo to give the title compound as a cream-coloured solid, 156 mg, 91% yield.

¹H NMR (400 MHz, DMSO-d₆) δ: 4.32 (s, 2H), 7.18-7.22 (m, 1H), 7.28-7.36 (m, 4H), 7.46-7.49 (m, 2H), 7.85-7.88 (m, 2H) ppm.

LRMS (ESI): m/z 259 [M—Cl+OMe]⁺ (sample prepared in methanol)

### Preparation 6

### 4-[(4-methoxybenzyl)thio]benzonitrile

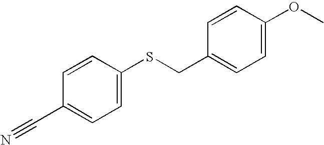

1-(bromomethyl)-4-methoxybenzene (137 mg, 0.68 mmol) was added to a solution of 4-mercaptobenzonitrile (128 mg, 0.95 mmol) in dimethylformamide (3.2 mL) before the addition of cesium carbonate (329 mg, 1.01 mmol). The solution was heated at 80° C. for 6 hours before stirring for 12 hours at ambient temperature. The resulting solution was poured into water (10 mL) and extracted with ethyl acetate (2×10 mL). The combined organic extracts were dried (magnesium sulphate) and concentrated in vacuo to give the title compound as a pale yellow semi-solid in a quantitative yield.

¹H NMR (400 MHz, DMSO-d₆) δ: 3.74 (s, 3H), 4.34 (s, 2H), 6.88-6.92 (m, 2H), 7.34-7.37 (m, 2H), 7.48-7.51 (m, 2H), 7.72-7.75 (m, 2H) ppm.

LRMS (API): m/z 256 [M+H]⁺

### Preparations 7-9

The following compounds were prepared by a method similar to that described for Preparation 6 using the appropriate starting materials in the presence of cesium carbonate. The reactions were monitored by TLC or LCMS analysis. When stated purification was undertaken by flash column chromatography on silica gel.

| | | | Yield | |
|---|---|---|---|---|
| No. | Structure | Name and NMR | (purified) | LRMS |
| 7 | 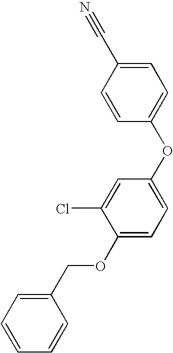 | 4-[4-(benzyloxy)-3- chlorophenoxy]benzonitrile Starting materials: 4-fluorobenzo-nitrile and 4- (benzyloxy)-3-chlorophenol as obtained in Preparation 45 ¹H NMR (400 MHz, DMSO-d₆) δ: 5.24(s, 2H), 7.08-7.11(m, 2H), 7.16(dd, 1H), 7.33-7.39(m, 3H), 7.42-7.46(m, 2H), 7.50-7.52(m, 2H), 7.83-7.86(m, 2H) ppm. | 89% (yes) | (ESI): m/z 335 [M + H]⁺ |
| 8 | 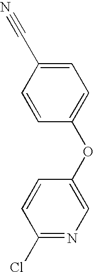 | 4-[(6-chloropyridin-3-yl)oxy] benzonitrile Starting materials: 4-fluorobenzo-nitrile and 6-chloropyridin-3-ol ¹H NMR (400 MHz, DMSO-d₆) δ: 7.23- 7.27(m, 2H), 7.62-7.64(m, 1H), 7.72- 7.75(m, 1H), 7.88-7.92(m, 2H), 8.36- 8.37(m, 1H) ppm. | 50% (yes) | (ESI): m/z 231 [M + H]⁺ |
| 9 | 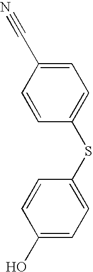 | 4-[(4-hydroxyphenyl)thio]-benzonitrile Starting materials: 4-fluorobenzo-nitrile and 4-mercaptophenol ¹H NMR (400 MHz, DMSO-d₆) δ: 6.91- 6.95(m, 2H), 7.12-7.15(m, 2H), 7.40- 7.44(m, 2H), 7.68-7.72(m, 2H), 10.07(s, 1H) ppm. | 56% (no) | (ESI): m/z 226 [M − H]⁻ |

### Preparation 10

### 4-[(4-methoxybenzyl)thio]benzoic acid

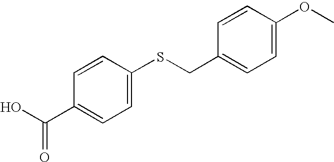

To a solution of 4-[(4-methoxybenzyl)thio]benzonitrile as obtained in Preparation 6 (259 mg, 1.01 mmol) in ethanol (8 mL) and water (4 mL) was added sodium hydroxide (413 mg, 10.30 mmol). The resulting solution was heated at reflux for 5 hours before stirring at ambient temperature for 8 hours. The resulting solution was poured into saturated sodium hydrogen carbonate solution (20 mL, aqueous) and extracted with ethyl acetate (2×30 mL). The aqueous extract was acidified to pH 3 by the addition of hydrochloric acid (2N aqueous solution) before being extracted with ethyl acetate (2×40 mL). Combined organic extracts were dried (magnesium sulphate) and concentrated in vacuo to give the title compound as a pink solid, 184 mg, 66% yield.

¹H NMR (400 MHz, DMSO-d₆) δ: 3.74 (s, 3H), 4.30 (s, 2H), 6.87-6.91 (m, 2H), 7.32-7.37 (m, 2H), 7.41-7.44 (m, 2H), 7.82-7.85 (m, 2H), 12.86 (br s, 1H) ppm.

LRMS (ESI): m/z 273 [M−H]⁻

LRMS (API): m/z 273 [M−H]⁻

### Preparations 11-15

The following compounds were prepared by a method similar to that described for Preparation 10 using the appropriate starting material in the presence of sodium hydroxide. The reactions were monitored by TLC or LCMS analysis. Compound 13 was purified by flash column chromatography on silica gel.

| No. | Structure | Name and NMR | Yield | LRMS |
|---|---|---|---|---|
| 11 | 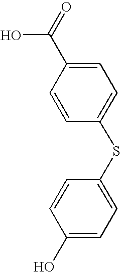 | 4-[(4-hydroxyphenyl)thio]benzoic acid Starting material: the compound as obtained in preparation 9 ¹H NMR (400 MHz, MeOD-d₆) δ: 6.90- 6.94(m, 2H), 7.08-7.12(m, 2H), 7.40- 7.44(m, 2H), 7.86-7.89(m, 2H) ppm. | 87% | (ESI): m/z 245 [M − H]⁻ |
| 12 | 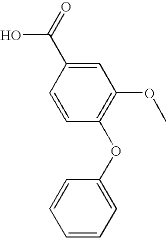 | 3-methoxy-4-phenoxybenzoic acid Starting material: the compound as obtained in preparation 23 ¹H NMR (400 MHz, CDCl₃) δ: 3.95(s, 3H), 6.86-6.91(m, 1H), 7.01-7.08(m, 2H), 7.13-7.20(m, 1H), 7.32-7.41(m, 2H), 7.66-7.76(m, 2H) ppm. | 88% | (ESI): m/z 243 [M − H]⁻ |
| 13 | 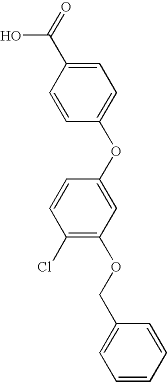 | 4-[3-(benzyloxy)-4-chlorophenoxy]- benzoic acid Starting material: the compound as obtained in preparation 25 ¹H NMR (400 MHz, DMSO-d₆) δ: 5.22(s, 2H), 6.71(dd, 1H), 7.03-7.07(m, 2H), 7.08-7.09(m, 1H), 7.34-7.47(m, 5H), 7.51(d, 1H), 7.94-7.98(m, 2H), 12.72(br s, 1H) ppm. | 47% | (ESI): m/z 353 [M − H]⁻ |
| 14 | 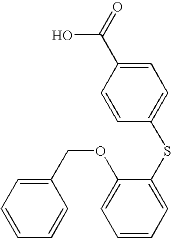 | 4-{[2-(benzyloxy)phenyl]thio}-benzoic acid Starting material: the compound as obtained in preparation 44. Methanol was employed as reaction solvent. ¹H NMR (400 MHz, CDCl₃) δ: 5.10(s, 2H), 7.01-7.05(m, 2H), 7.15-7.30(m, 7H), 7.38-7.42(m, 1H), 7.52-7.55(m, 1H), 7.95-7.98(m, 2H) ppm. | 99% | (ESI): m/z 335 [M − H]⁻ |
| 15 | 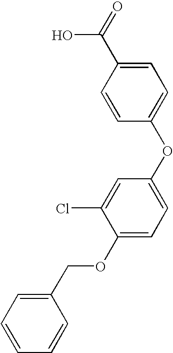 | 4-[4-(benzyloxy)-3-chlorophenoxy]- benzoic acid Starting material: the compound as obtained in preparation 7 ¹H NMR (400 MHz, DMSO-d₆) δ: 5.24(s, 2H), 7.00-7.04(m, 2H), 7.11-7.15(m, 1H), 7.32-7.40(m, 3H), 7.42-7.46(m, 2H), 7.50-7.52(m, 2H), 7.94-7.97(m, 2H), 12.83(br s, 1H) ppm. | 82% | (ESI): m/z 354 [M + H]⁺ 352 [M − H]⁻ |

### Preparation 16

### 4-[(4-hydroxybenzyl)thio]benzoic acid

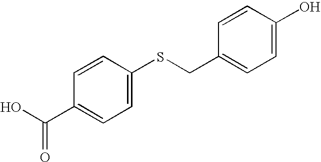

A solution of 4-[(4-methoxybenzyl)thio]benzoic acid as obtained in Preparation 10 (182 mg, 0.66 mmol) in dichloromethane (2 mL) was cooled over ice before the dropwise addition of boron tribromide (282 μL, 2.98 mmol). The reaction was slowly warmed to ambient temperature and stirred for 12 hours before being cooled over ice. Ice (15 mL) was added and the mixture was warmed to ambient temperature before the resulting solid was filtered off. The solid was stirred vigorously in sodium hydroxide solution (2.5N aqueous, 6 mL) for 3 hours. The reaction mixture was filtered and the filtrate was acidified to pH 1 by addition of hydrochloric acid (2N aqueous solution). The resulting solution was extracted with ethyl acetate (4×20 mL). The combined organic extracts were dried (magnesium sulphate), concentrated in vacuo and purified by flash column chromatography on silica gel eluting with ethyl acetate:heptane (0:1 to 1:0, by volume, gradient elution) to give the title compound as a pink solid, 129 mg, 75% yield.

¹H NMR (400 MHz, DMSO-d₆) δ: 4.23 (s, 2H), 6.69-6.73 (m, 2H), 7.19-7.23 (m, 2H), 7.36-7.40 (m, 2H), 7.81-7.84 (m, 2H) ppm.

LRMS (ESI): m/z 261 [M+H]⁺259 [M−H]⁻

### Preparations 17-19

The following compounds were prepared by a method similar to that described for Preparation 16 using the appropriate starting material in the presence of a solution of boron tribromide in dichloromethane. The reactions were monitored by TLC or LCMS analysis. When stated, purification was by flash column chromatography on silica gel.

| No. | Structure | Name and NMR | Yield | LRMS |
|---|---|---|---|---|
| 17 | 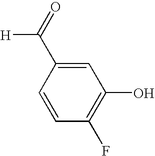 | 4-fluoro-3-hydroxybenz-aldehyde Starting material: 4-fluoro-3- methoxybenzaldehyde ¹H NMR (400 MHz, CDCl₃) δ: 5.46 (m, 1H), 7.23-7.28(m, 1H), 7.44-7.48 (m, 1H), 7.55-7.57(m, 1H), 9.92(s, 1H) ppm. | 38% | (ESI): m/z 282 2 [M + H]⁺ |
| 18 | 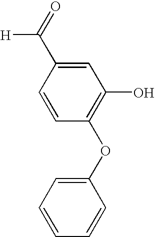 | 3-hydroxy-4-phenoxybenzoic acid Starting material: the compound as obtained in preparation 12 ¹H NMR (400 MHz, DMSO-d₆) δ: 6.94-6.98(m, 3H), 7.09-7.13(m, 1H), 7.35-7.40(m, 2H), 7.41-7.44(m, 1H), 7.56-7.57(m, 1H), 9.94(br s, 1H) ppm. | 64% (purified) | (ESI): m/z 229 [M − H]⁻ |
| 19 | 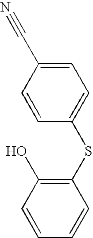 | 4-[(2-hydroxyphenyl)thio]-benzonitrile Starting material: the compound as obtained in preparation 24 ¹H NMR (400 MHz, DMSO-d₆) δ: 7.00- 7.05(m, 1H), 7.07-7.10(m, 2H), 7.12- 7.14(m, 1H), 7.44-7.53(m, 4H) ppm. | 97% | (ESI): m/z 226 [M − H]⁻ |

### Preparation 20

### 4-(3-chloro-4-hydroxyphenoxy)benzoic acid

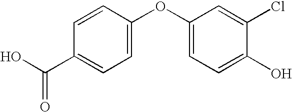

A suspension of 4-[4-(benzyloxy)-3-chlorophenoxy]benzoic acid as obtained in Preparation 15 (500 mg, 1.41 mmol) in dichloromethane (18 mL) was cooled to −60° C. (acetone/solid CO₂) under nitrogen before the dropwise addition of boron tribromide (1M solution in dichloromethane, 2.96 mL, 2.96 mmol). The reaction temperature was warmed to −40° C. for 4 hours before quenching with water (5 mL) at −10° C. The reaction mixture was warmed to ambient temperature and diluted with dichloromethane (50 mL), water (10 mL) and hydrochloric acid (0.2N aqueous solution, 5 mL). The layers were separated and the aqueous layer was extracted with dichloromethane (2×20 mL). The combined organic extracts were dried (magnesium sulphate) and concentrated in vacuo. The residue was purified by flash column chromatography on silica gel eluting with dichloromethane:methanol:acetic acid (200:0:1 to 190:10:1, by volume, gradient elution) to give the title compound as a solid, 330 mg, 89% yield.

¹H NMR (400 MHz, CDCl₃) δ: 6.94-6.99 (m, 3H), 7.05-7.07 (m, 1H), 7.12 (d, 1H), 8.06-8.09 (m, 2H) ppm. LRMS (ESI): m/z 263 [M−H]⁻

### Preparation 21

### 4-{[(4-hydroxyphenyl)thio]methyl}benzoic acid

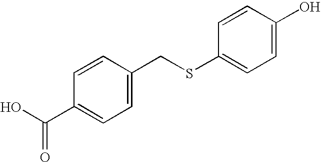

A solution of 4-mercaptophenol (334 mg, 2.57 mmol) and methyl 4-(bromomethyl)benzoate (600 mg, 2.60 mmol) in 1,4-dioxane (20 mL) was treated with triethylamine (718 μL, 5.13 mmol) at ambient temperature. After stirring for 6 hours the resulting solution was poured into saturated ammonium chloride solution (40 mL, aqueous) and extracted with ethyl acetate (3×40 mL). The combined organic extracts were washed with brine (60 mL), dried (sodium sulphate) and concentrated in vacuo to give a pale yellow solid. This residue was dissolved in a mixture of tetrahydrofuran (20 mL) and 1,4-dioxane (5 mL) and treated with water (4 mL) and lithium hydroxide (2.19 g, 51.20 mmol). The resulting solution was heated to 60° C. for 1 hour and then allowed to cool to ambient temperature. After stirring at ambient temperature for 18 hours the solution was concentrated in vacuo. The residue was dissolved in water (20 mL), cooled to 0° C. and the pH of the solution adjusted to 5.5 by the addition of acetic acid. The resulting suspension was filtered to give a white solid which was dissolved in methanol (50 mL) and concentrated in vacuo to give the title compound as a white solid, 612 mg, 87% yield.

¹H NMR (400 MHz, DMSO-d₆) δ: 4.03 (s, 2H), 6.70-6.74 (m, 2H), 7.12-7.16 (m, 4H), 7.76-7.78 (m, 2H) ppm.

LRMS (ESI): m/z 261 [M+H]⁺

### Preparation 22

### 4-{[6-(benzyloxy)pyridin-3-yl]oxy}benzoic acid

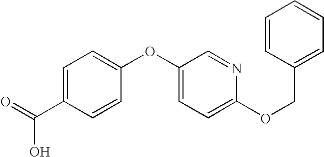

To a suspension of 4-[(6-chloropyridin-3-yl)oxy]benzonitrile as obtained in Preparation 8 (475 mg, 2.06 mmol) in toluene (10 mL) was added phenylmethanol (235 μL, 2.27 mmol), potassium hydroxide (231 mg, 4.12 mmol) and 1,4,7,10,13,16-hexaoxacyclooctadecane (27 mg, 103 μmol). The solution was heated at 100° C. for 24 hours and then at ambient temperature for 60 hours. Phenylmethanol (214 μL, 2.07 mmol) was added and the solution was heated at 100° C. for 24 hours. Potassium hydroxide (231 mg, 4.12 mmol) was added and the solution was heated at 100° C. for 24 hours. The solution was diluted with water (5 mL) and heated at 100° C. for 24 hours and then at ambient temperature for 24 hours. The solution was concentrated in vacuo and diluted with aqueous sodium hydroxide (1M, 5 ml) and heated at 100° C. for 60 hours. After cooling to ambient temperature the resulting mixture was poured into water (10 mL) and extracted with dichloromethane (2×20 mL). The combined organic extracts were dried (magnesium sulphate) and concentrated in vacuo. The residue was purified by flash column chromatography on silica gel eluting with ethyl acetate:heptane:acetic acid (10:90:1 to 80:20:1, by volume, gradient elution) to afford a white solid. This residue was crystallised from hot ethyl acetate:heptane (1:1) to give the title compound as a white crystalline solid, 56 mg, 9% yield.

¹H NMR (400 MHz, DMSO-d₆) δ: 5.35 (s, 2H), 6.97-7.03 (m, 3H), 7.30-7.42 (m, 3H), 7.45-7.49 (m, 2H), 7.63 (dd, 1H), 7.91-7.95 (m, 2H), 8.09 (d, 1H), 12.77 (br s, 1H) ppm.

LRMS (ESI): m/z 322 [M+H]⁺

### Preparation 23

### 3-methoxy-4-phenoxybenzonitrile

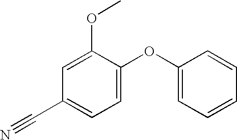

To a solution of 4-fluoro-3-methoxybenzonitrile (500 mg, 3.00 mmol) in acetonitrile (8 mL) was added phenol (305 mg, 3.24 mmol), 2-hydroxybenzaldehyde oxime (89 mg, 0.65 mmol), cesium carbonate (2.11 g, 6.49 mmol) and copper (I) oxide (23 mg, 0.16 mmol). The reaction mixture was degassed and heated at 80° C. for 24 hours before being stirred at ambient temperature for 36 hours. The solution was poured into water (100 mL) and ethyl acetate (50 mL) and was acidified to pH 2 by the addition of hydrochloric acid (0.2N aqueous solution). The aqueous phase was extracted with ethyl acetate (50 mL) and the combined organic phases were washed with saturated sodium hydrogen carbonate solution (30 ml, aqueous), dried (magnesium sulphate) and concentrated in vacuo. The residue was purified by flash column chromatography on silica gel eluting with ethyl acetate:heptane (0:1 to 2:8, by volume, gradient elution) to give the title compound as a white solid, 422 mg, 58% yield.

¹H NMR (400 MHz, CDCl₃) δ: 3.93 (s, 3H), 6.86-6.88 (m, 1H), 7.01-7.04 (m, 2H), 7.14-7.22 (m, 3H), 7.36-7.41 (m, 2H) ppm.

LRMS (ESI): m/z 226 [M+H]⁺

### Preparation 24

### 4-[(2-methoxyphenyl)thio]benzonitrile

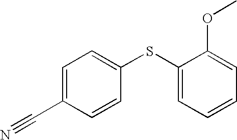

The title compound was prepared by a method similar to that described for Preparation 23 using 4-fluorobenzo-nitrile and 2-methoxybenzenethiol as starting materials, copper (I) oxide, 2-hydroxybenzaldehyde oxime and cesium carbonate in acetonitrile. The reaction was monitored by TLC or LCMS analysis. Purification was undertaken by flash column chromatography on silica gel eluting with dichloromethane:heptane 3:7 to give the title compound, 56% yield.

¹H NMR (400 MHz, CDCl₃) δ: 3.83 (s, 3H), 7.01-7.05 (m, 2H), 7.11-7.14 (m, 2H), 7.44-7.51 (m, 4H) ppm. (ESI): m/z 242 [M+H]⁺

### Preparation 25

### 4-[3-(benzyloxy)-4-chlorophenoxy]benzonitrile

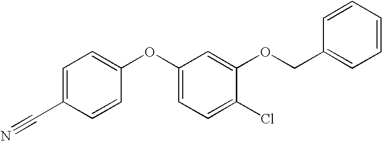

A solution of 3-(benzyloxy)-4-chlorophenol as obtained in Preparation 43 (900 mg, 3.80 mmol), 4-iodobenzonitrile (878 mg, 3.84 mmol), tripotassium phosphate (1.63 g, 7.67 mmol), copper (I) iodide (73 mg, 384 μmol) and N,N,N-tributylbutan-1-aminium bromide (124 mg, 384 μmol) in N,N-dimethylformamide (19 mL) was degassed and heated at 110° C. for 5 days. After cooling to ambient temperature the resulting solution was poured into water (200 mL), acidified to approximately pH 3 by addition of hydrochloric acid (2N aqueous solution) and extracted with ethyl acetate (3×100 mL). The combined organic extracts were washed with brine (3×300 mL), dried (magnesium sulphate) and concentrated in vacuo. The residue was purified by flash column chromatography on silica gel eluting with ethyl acetate:heptane (1:7, by volume) to afford the title compound as a colourless solid, 600 mg, 47% yield.

¹H NMR (400 MHz, CDCl₃) δ: 5.13 (s, 2H), 6.60-6.63 (m, 1H), 6.68-6.69 (m, 1H), 6.94-6.97 (m, 2H), 7.34-7.44 (m, 6H), 7.57-7.61 (m, 2H) ppm.

LRMS (ESI): m/z 334 [M−H]⁻

### Preparation 26

### 4-{[(4-acetoxyphenyl)thio]methyl}benzoic acid

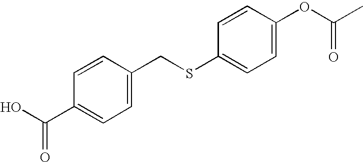

4-{[(4-hydroxyphenyl)thio]methyl}benzoic acid as obtained in Preparation 21 (140 mg, 0.54 mmol) was suspended in pyridine (209 μL, 2.58 mmol) and acetic anhydride (203 μL, 2.15 mmol). After stirring at ambient temperature for 18 hours the resulting suspension was treated with dimethylformamide (0.5 mL). After stirring for 24 hours the resulting suspension was diluted with water (2 mL), acidified to pH 2 by the addition of concentrated hydrochloric acid and extracted with ethyl acetate (4×10 mL). The combined organic extracts were dried (magnesium sulphate) and concentrated in vacuo to give the title compound as a cream solid, 192 mg, quantitative yield.

¹H NMR (400 MHz, DMSO-d₆) δ: 2.26 (s, 3H), 4.32 (s, 2H), 7.06-7.10 (m, 2H), 7.36-7.39 (m, 2H), 7.44-7.47 (m, 2H), 7.85-7.89 (m, 2H), 12.05 (br s, 1H) ppm.

LRMS (ESI): m/z 301 [M−H]⁻

### Preparations 27-33

The following compounds were prepared by a method similar to that described for Preparation 26 using the appropriate starting material and acetic anhydride in the presence of pyridine. The reactions were monitored by TLC or LCMS analysis. When stated, purification was undertaken by flash column chromatography on silica gel.

| | | | Yield | |
|---|---|---|---|---|
| No | Structure | Namd and NMR | (purified?) | LRMS |
| 27 | 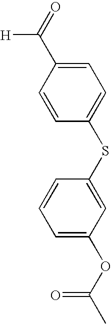 | 3-[(4-formylphenyl)thio]phenyl acetate Starting material: the compound as obtained in preparation 34 ¹H NMR (400 MHz, CDCl₃) δ: 2.31(s, 3H), 7.14-7.17(m, 1H), 7.25-7.26(m, 1H), 7.30-7.33(m, 2H), 7.36-7.39(m, 1H), 7.40-7.46(m, 1H), 7.75-7.78(m, 2H), 9.94(s, 1H) ppm. | 64% (yes) | (ESI): m/z 273 [M + H]⁺ |
| 28 | 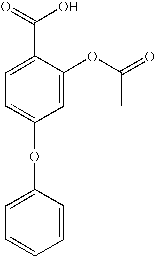 | 2-acetoxy-4-phenoxybenzoic acid Starting material: 2-acetoxy-4- phenoxybenzoic acid (Ungnade H, Ortega I, Journal of Organic Chemistry, 2002, pages 1475-1483) ¹H NMR (400 MHz, CDCl₃) δ: 2.32(s, 3H), 6.67(m, 1H), 6.88-6.91(m, 1H), 7.10-7.13(m, 2H), 7.23-7.25(m, 1H), 7.41-7.46(m, 2H), 8.09(d, 1H) ppm. | 99% (no) | (ESI): m/z 271 [M + H]⁺ |
| 29 | 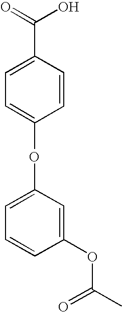 | 4-(3-acetoxyphenoxy)benzoic acid Starting material: 4-(3- hydroxyphenoxy)benzoic acid ¹H NMR (400 MHz, CDCl₃) δ: 2.30(s, 3H), 6.84-6.85(m, 1H), 6.94-6.98(m, 2H), 7.05-7.08(m, 2H), 7.38-7.42(m, 1H), 8.09-8.12(m, 2H) ppm. | 99% (no) | (ESI): m/z 271 [M − H]⁻ |
| 30 | 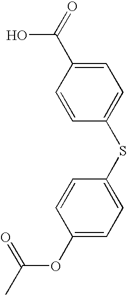 | 4-[(4-acetoxyphenyl)thio]-benzoic acid Starting material: the compound as obtained in preparation 11 ¹H NMR (400 MHz, CDCl₃) δ: 2.33(s, 3H), 7.15-7.18(m, 2H), 7.21-7.24(m, 2H), 7.51-7.55(m, 2H), 7.95-7.98(m, 2H) ppm. | 91% (no) | (ESI): m/z 287 [M − H]⁻ |
| 31 | 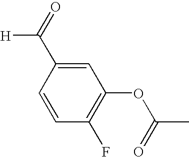 | 2-fluoro-5-formylphenyl acetate Starting material: the compound as obtained in preparation 17 ¹H NMR (400 MHz, CDCl₃) δ: 2.38(s, 3H), 7.32-7.36(m, 1H), 7.70-7.73(m, 1H), 7.77-7.81(m, 1H), 9.95(s, 1H) ppm. | 80% (yes) | (ESI): m/z 183 [M + H]⁺ |
| 32 | 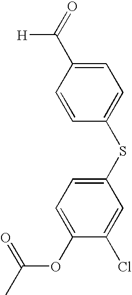 | 2-chloro-4-[(4-formylphenyl)-thio]phenyl acetate Starting material: the compound as obtained in preparation 35 ¹H NMR (400 MHz, CDCl₃) δ: 2.38(s, 3H), 7.20(d, 1H), 7.31-7.34(m, 2H), 7.40- 7.42(m, 1H), 7.59(d, 1H), 7.77-7.80(m, 2H), 9.95(s, 1H) ppm. | 100% (yes) | (ESI): m/z 307 [M + H]⁺ |
| 33 | 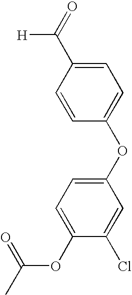 | 4-(4-acetoxy-3-chlorophenoxy)benzoic acid Starting material: the compound as obtained in preparation 20 ¹H NMR (400 MHz, CDCl₃) δ: 2.38(s, 3H), 6.99-7.02(m, 1H), 7.04-7.08(m, 2H), 7.15-7.18(m, 2H), 8.10-8.14(m, 2H) ppm. | 85% (no) | (ESI): m/z 305 [M − H]⁻ |

### Preparation 34

### 4-[(3-hydroxyphenyl)thio]benzaldehyde

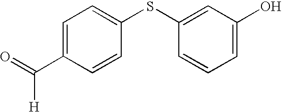

A suspension of potassium carbonate (1.31 g, 9.48 mmol) in dimethylformamide (6 mL) was degassed 3 times before the addition of 3-mercaptophenol (500 μL, 4.90 mmol) and 4-fluorobenzaldehyde (500 μL, 4.74 mmol). The reaction mixture was degassed twice before being stirred at ambient temperature for 16 hours. The resulting suspension was diluted with ethyl acetate (30 mL) and acidified to approximately pH 2 by the addition of hydrochloric acid (1N aqueous solution, 6 mL). The aqueous phase was extracted with ethyl acetate (2×30 mL) and the combined organic extracts were washed with brine (20 mL), dried (magnesium sulphate) and concentrated in vacuo. The residue was purified by flash column chromatography on silica gel eluting with heptane:ethyl acetate (5:1, by volume) to give the title compound as an oil, 747 mg, 69% yield.

¹H NMR (400 MHz, DMSO-d₆) δ: 6.87-6.92 (m, 2H), 6.96-6.98 (m, 1H), 7.30-7.41 (m, 3H), 7.83-7.85 (m, 2H), 9.84 (s, 1H), 9.94 (s, 1H) ppm.

LRMS (ESI): m/z 231 [M+H]⁺

### Preparation 35

### 4-[(3-chloro-4-hydroxyphenyl)thio]benzaldehyde

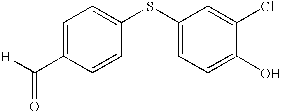

The title compound was prepared by a method similar to that described for Preparation 34 using p-fluorobenzaldehyde and 2-chloro-4-mercaptophenol as obtained in preparation 68 as starting materials in the presence of potassium carbonate. The reaction was monitored by TLC or LCMS analysis. The title compound was obtained with a yield of 72%.

¹H NMR (400 MHz, CDCl₃) δ: 5.83 (br s, 1H), 7.11 (d, 1H), 7.17-7.20 (m, 2H), 7.40 (dd, 1H), 7.57 (d, 1H), 7.72-7.75 (m, 2H), 9.92 (s, 1H) ppm.

(ESI): m/z 263 [M−H]⁻ 265 [M+H]⁺

### Preparation 36

### 5-formyl-2-(phenylthio)phenyl acetate

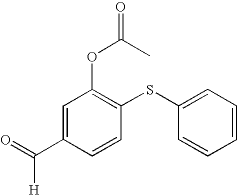

Potassium carbonate (1.58 g, 11.40 mmol) was added to DMF (7 mL) and degassed 3 times before the addition of thiophenol (800 μL, 7.82 mmol). The mixture was subsequently degassed for one minute prior to the addition of 2-fluoro-5-formylphenyl acetate as obtained in Preparation 31 (1.30 g, 5.70 mmol). The mixture was degassed 3 times and stirred at ambient temperature for 48 hours. The resulting suspension was diluted with ethyl acetate (50 mL) and water (100 mL) and acidified to pH 2 by addition of hydrochloric acid (0.2N aqueous solution). The aqueous phase was extracted with ethyl acetate (2×50 mL) and the combined organic extracts were washed with water (2×100 mL) and concentrated in vacuo. The residue was dissolved in pyridine (1 mL, 3.00 mmol) and dichloromethane (5 mL) and stirred at ambient temperature under nitrogen. Acetic anhydride (443 μL, 4.70 mmol) was added dropwise and the reaction was stirred at ambient temperature for 2.5 hours. The suspension was diluted with water (50 mL) and acidified to pH 2 by addition of hydrochloric acid (2N aqueous solution). The aqueous phase was extracted with ethyl acetate (3×50 mL) and the combined organic extracts were washed with brine (50 mL), dried (magnesium sulphate) and concentrated in vacuo to give the title compound as an oil, 480 mg, 31% yield.

¹H NMR (400 MHz, CDCl₃) δ: 2.37 (s, 3H), 7.02 (d, 1H), 7.42-7.46 (m, 3H), 7.50-7.59 (m, 4H), 9.91 (s, 1H) ppm.

LRMS (ESI): m/z 273 [M+H]⁺

### Preparation 37

### 4-[(3-acetoxyphenyl)thio]benzoic acid

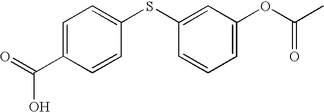

A solution of 3-[(4-formylphenyl)thio]phenyl acetate as obtained in Preparation 27 (300 mg, 1.10 mmol) in acetonitrile (11 mL) was treated with copper(I) chloride (6.5 mg, 66 μmol) and cooled to 0° C. The resulting solution was treated with tert-butyl hydroperoxide (70% solution in water, 0.2 mL, 1.54 mmol). After stirring for 24 hours the resulting solution was concentrated in vacuo and the residue was treated with saturated aqueous sodium hydrogen carbonate solution (50 mL, aqueous) then acidified to pH 2 by the addition of hydrochloric acid (2N aqueous solution) and extracted with ethyl acetate (3×50 mL). The combined organic extracts were dried (magnesium sulphate) and concentrated in vacuo. The residue was purified by flash column chromatography on silica gel eluting with heptane:ethyl acetate:acetic acid (140:60:1, by volume) to give the title compound as an oil, 200 mg, 63% yield.

¹H NMR (400 MHz, CDCl₃) δ: 2.30 (s, 3H), 7.11-7.14 (m, 1H), 7.23-7.24 (m, 1H), 7.27-7.29 (m, 2H), 7.33-7.38 (m, 1H), 7.39-7.43 (m, 1H), 7.97-8.00 (m, 2H) ppm.

LRMS (ESI): m/z 287 [M−H]⁻

### Preparations 38-39

The following compounds were prepared by a method similar to that described for Preparation 37 using the appropriate starting material and tert-butyl hydroperoxide in the presence of copper (I) chloride. The reactions were monitored by TLC or LCMS analysis.

| No | Structure | Name and NMR | Yield | LRMS |
|---|---|---|---|---|
| 38 | 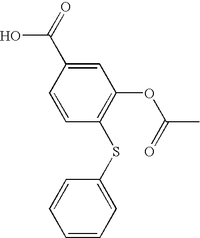 | 3-acetoxy-4-(phenylthio)-benzoic acid Starting material: the compound as obtained in preparation 36 ¹H NMR (400 MHz, CDCl₃) δ: 2.35(s, 3H), 6.98-7.01(m, 1H), 7.40-7.45(m, 3H), 7.48-7.54(m, 2H), 7.77-7.80(m, 2H) ppm. | 45% | (ESI): m/z 287 [M − H]⁻ |
| 39 | 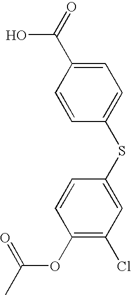 | 4-[(4-acetoxy-3-chlorophenyl): thio]benzoic acid Starting material: the compound as obtained in preparation 32 ¹H NMR (400 MHz, CDCl₃) δ: 2.38(s, 3H), 7.17-7.19(m, 1H), 7.28-7.31(m, 2H), 7.38-7.40(m, 1H), 7.57(d, 1H), 7.99-8.03(m, 2H) ppm. | 73% | (ESI): m/z 321 [M − H]⁻ |

### Preparation 40

### methyl 4-{[3-(methylthio)phenyl]thio}benzoate

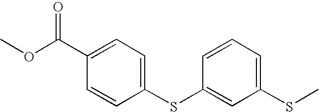

A suspension of [4-(methoxycarbonyl)phenyl]boronic acid (2.30 g, 12.80 mmol) and copper (I) iodide (61 mg, 0.32 mmol) in dimethylsulphoxide (30 mL) was treated with 2,2′-bipyridine (51 mg, 0.32 mmol) followed by 3-(methylthio)benzenethiol [Rumpf P., Bulletin de la societe Chimique de France, 1940, pages 632-634] (1.00 g, 6.40 mmol) and water (7 mL). The resulting solution was stirred whilst open to air and heated to 100° C. After heating for 16 hours the resulting solution was allowed to cooled to ambient temperature and diluted with hydrochloric acid (1N aqueous solution, 75 mL) and extracted with ethyl acetate (3×50 mL). The combined organic extracts were washed with water (50 mL), brine (50 mL), dried (sodium sulphate) and concentrated in vacuo. The residue was purified by flash column chromatography on silica gel eluting with heptane:ethyl acetate (19:1 to 1:1, by volume, gradient elution) to give the title compound as a white solid, 1.25 g, 67% yield.

¹H NMR (400 MHz, DMSO-d₆) δ: 2.49 (s, 3H), 3.85 (s, 3H), 7.24-7.27 (m, 1H), 7.30-7.36 (m, 4H), 7.39-7.44 (m, 1H), 7.89-7.92 (m, 2H) ppm.

LRMS (ESI): m/z 291 [M+H]⁺

### Preparation 41

### 4-{[3-(methylthio)phenyl]thio}benzoic acid

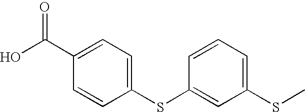

A solution of methyl 4-{[3-(methylthio)phenyl]thio}benzoate as obtained in Preparation 40 (1.25 g, 4.30 mmol) in tetrahydrofuran (50 mL) and 1,4-dioxane (15 mL) was treated with water (15 mL) followed by lithium hydroxide monohydrate (3.50 g, 81.80 mmol). The resulting suspension was stirred and heated to 60° C. for 1.5 hours and after cooling to ambient temperature concentrated in vacuo. The residue was suspended in water (40 mL) and cooled to 0° C. and the pH adjusted to 5.5 by the addition of acetic acid. The resulting suspension was filtered to collect a white solid which was washed with water (40 mL), suspended in methanol (40 mL) and concentrated in vacuo to give the title compound as a white solid, 1.19 g, 100% yield.

¹H NMR (400 MHz, DMSO-d₆) δ: 2.49 (s, 3H), 7.22-7.25 (m, 1H), 7.29-7.34 (m, 4H), 7.39-7.43 (m, 1H), 7.88-7.91 (m, 2H), 13.03 (br s, 1H) ppm.

LRMS (ESI): m/z 275 [M−H]⁻

### Preparation 42

### benzyl 3-(benzyloxy)-4-phenoxybenzoate

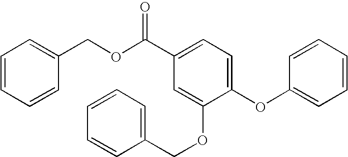

To a solution of 3-hydroxy-4-phenoxybenzoic acid as obtained in Preparation 18 (400 mg, 1.74 mmol) in anhydrous dimethylformamide (8 mL) was added N-ethyl-N-isopropylpropan-2-amine (666 μL, 3.82 mmol) and bromomethylbenzene (217 μL, 1.82 mmol). After stirring at ambient temperature for 20 hours the solution was treated with N-ethyl-N-isopropylpropan-2-amine (666 μL, 3.82 mmol) and bromomethylbenzene (217 μL, 1.82 mmol) and stirred at ambient temperature for 20 hours. Further bromomethylbenzene (413 μL, 3.46 mmol) was added and reaction mixture was stirred at ambient temperature for 60 hours. N-ethyl-N-isopropylpropan-2-amine (605 μL, 3.47 mmol) and bromomethylbenzene (413 μL, 3.46 mmol) were added and the solution was stirred at ambient temperature for 24 hours. The reaction was diluted with water (20 mL) before being extracted with ethyl acetate (20 mL). The aqueous phase was extracted with ethyl acetate (3×20 mL) and the combined organic extracts were washed with water (3×80 mL), dried (magnesium sulphate) and concentrated in vacuo. The residue was purified by flash column chromatography on silica gel eluting with heptane:ethyl:acetate (95:5, by volume) to give the title compound as a colourless solid, 700 mg, 98% yield.

¹H NMR (400 MHz, CDCl₃) δ: 5.19 (s, 2H), 5.39 (s, 2H), 7.01-7.04 (m, 3H), 7.14-7.18 (m, 1H), 7.28-7.49 (m, 12H), 7.72 (dd, 1H), 7.80 (d, 1H) ppm.

LRMS (ESI): m/z 409 [M−H]⁻

### Preparation 43

### 3-(benzyloxy)-4-chlorophenol

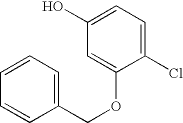

To an ice cooled solution of 4-chlorobenzene-1,3-diol (6.00 g, 20.80 mmol) in 2-propanone (25 mL) was added potassium carbonate (11.50 g, 83.00 mmol) portionwise. Bromomethylbenzene (4.04 mL, 34.00 mmol) was added dropwise before the reaction mixture was warmed to ambient temperature under nitrogen. After stirring at ambient temperature for 84 hours water (100 mL) and ethyl acetate (100 mL) were added. The aqueous phase was extracted with ethyl acetate (100 mL) and the combined organic extracts were washed with brine (200 mL), dried (magnesium sulphate) and concentrated in vacuo. The residue was purified by flash column chromatography on silica gel eluting with heptane:ethyl acetate (6:1, by volume) to give the title compound as a pale orange oil, 5.43 g, 55% yield.

¹H NMR (400 MHz, CDCl₃) δ: 5.12 (s, 2H), 6.38 (dd, 1H), 6.51 (d, 1H), 7.22 (d, 1H), 7.31-7.36 (m, 1H), 7.38-7.42 (m, 2H), 7.46-7.48 (m, 2H) ppm.

LRMS (ESI): m/z 233 [M−H]⁻

### Preparations 44-45

The following compounds were prepared by a method similar to that described for Preparation 43 using the appropriate starting material and bromomethylbenzene heated at reflux in 2-propanone in the presence of potassium carbonate. The reactions were monitored by TLC or LCMS analysis.

| No | Structure | Name and NMR | Yield | LRMS |
|---|---|---|---|---|
| 44 | 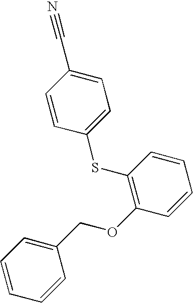 | 4-{[2-(benzyloxy)phenyl]thio} benzonitrile Starting material: the compound as obtained in preparation 19 ¹H NMR (400 MHz, CDCl₃) δ: 5.07 (s, 2H), 7.00-7.05(m, 2H), 7.12- 7.29(m, 7H), 7.39-7.46(m, 3H), 7.52-7.54(m, 1H) ppm. | 73% (no purification undertaken) | |
| 45 | 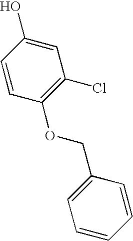 | 4-(benzyloxy)-3-chlorophenol Starting material: 2-chlorobenzene- 1,4-diol ¹H NMR (400 MHz, DMSO-d₆) δ: 5.09(s, 2H), 6.69(dd, 1H), 6.85(d, 1H), 7.06(d, 1H), 7.32-7.36(m, 1H), 7.39-7.43(m, 2H), 7.45-7.48 (m, 2H), 9.40(br s, 1H) ppm. | 35% | (ESI): m/z 233 [M − H]⁻ |

### Preparation 46

### 3-(benzyloxy)-4-phenoxybenzoic acid

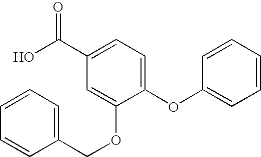

To a suspension of benzyl 3-(benzyloxy)-4-phenoxybenzoate as obtained in Preparation 42 (700 mg, 1.70 mmol) in ethanol (15 mL) and water (15 mL) was added sodium hydroxide (614 mg, 15.30 mmol). The resulting suspension was heated at reflux for 4 hours before cooling to ambient temperature. Water (50 mL) was added and the mixture was acidified by addition of hydrochloric acid (2N aqueous solution, 25 mL) before being extracted with ethyl acetate (50 mL). The aqueous phase was extracted with ethyl acetate (3×50 mL) and the combined organic extracts were washed with water (150 mL), dried (magnesium sulphate) and concentrated in vacuo. The residue was purified by flash column chromatography on silica gel eluting with heptane:ethyl acetate (9:1, by volume) to give the title compound as a colourless solid, 533 mg, 98% yield.

¹H NMR (400 MHz, CDCl₃) δ: 5.19 (s, 2H), 6.99-7.04 (m, 3H), 7.13-7.18 (m, 1H), 7.28-7.39 (m, 7H), 7.71-7.74 (m, 1H), 7.79-7.80 (m, 1H) ppm.

LRMS (ESI): m/z 319 [M−H]⁻

### Preparation 47

### (11beta,17alpha)-17-{[4-(4-acetoxyphenoxy)benzoyl]oxy}-9-fluoro-11-hydroxy-3-oxoandrosta-1,4-diene-17-carboxylic acid

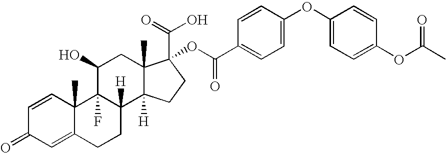

A solution of 4-(4-hydroxyphenoxy)benzoic acid (240 mg, 1.04 mmol) in pyridine (404 μL, 4.99 mmol) was treated with dropwise addition of acetic anhydride (394 μL, 4.17 mmol). After stirring at ambient temperature for 45 minutes the solution was diluted with water (40 mL), acidified to pH 2 by addition of concentrated hydrochloric acid and extracted with ethyl acetate (2×50 mL). The combined organic extracts were washed with brine (20 mL), dried (magnesium sulphate) and concentrated in vacuo. The residue was dissolved in dichloromethane (5.2 mL), treated with dimethylformamide (4 μL) and cooled to 0° C. The resulting solution was treated with oxalyl chloride (305 μL, 3.50 mmol) and allowed to warm to ambient temperature. After stirring for 12 hours at ambient temperature the solution was concentrated in vacuo before being diluted with acetone (4 mL). This solution was added dropwise over 2 minutes to a cooled suspension (0° C.) of (11beta,17alpha)-9-fluoro-11,17-dihydroxy-3-oxoandrosta-1,4-diene-17-carboxylic acid [Phillipps et al, Journal of Medicinal Chemistry, 1994, pages 3717-3729] (303 mg, 0.83 mmol) and pyridine (84 μL, 1.04 mmol) in acetone (6 mL). The reaction was stirred at ambient temperature for 6 hours then cooled to 0° C. before the addition of diethylamine (258 μL, 2.50 mmol). The reaction was stirred at ambient temperature for 12 hours and concentrated in vacuo before being diluted with ethyl acetate (20 mL). The organic extract was washed with water (30 mL). The aqueous extract was acidified to pH 1 by the addition of hydrochloric acid (2N aqueous solution) and extracted with ethyl acetate (2×50 mL). The combined organic extracts were dried (sodium sulphate) and concentrated in vacuo. The residue was purified by flash column chromatography on silica gel eluting with heptane:dichloromethane:ethyl acetate:acetic acid (80:20:100:1, by volume) to afford the title compound as a white solid, 313 mg, 49% yield.

¹H NMR (400 MHz, CDCl₃) δ: 1.16 (s, 3H), 1.47-1.68 (m, 2H), 1.58 (s, 3H), 1.75-1.82 (m, 2H), 1.90-1.96 (m, 1H), 2.01-2.12 (m, 1H), 2.24-2.32 (m, 1H), 2.31 (s, 3H), 2.40-2.57 (m, 3H), 2.62-2.70 (m, 1H), 2.97-3.05 (m, 1H), 4.46-4.49 (m, 1H), 6.16 (m, 1H), 6.38 (dd, 1H), 6.95-6.99 (m, 2H), 7.02-7.05 (m, 2H), 7.08-7.12 (m, 2H), 7.24 (d, 1H), 7.89-7.93 (m, 2H) ppm.

LRMS (ESI) 619 [M+H]⁺617 [M−H]⁻

### Preparation 48

### cyanomethyl(11beta,17alpha)-17-{[4-(4-acetoxyphenoxy)benzoyl]oxy}-9-fluoro-11-hydroxy-3-oxoandrosta-1,4-diene-17-carboxylate

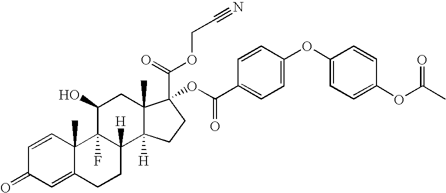

To a solution of (11beta,17alpha)-17-{[4-(4-acetoxyphenoxy)benzoyl]oxy}-9-fluoro-11-hydroxy-3-oxoandrosta-1,4-diene-17-carboxylic acid as obtained in Preparation 47 (313 mg, 0.51 mmol) in dimethylformamide (3 mL) was added sodium hydrogen carbonate (50 mg, 0.59 mmol) and bromoacetonitrile (36 μL, 0.54 mmol). After stirring at ambient temperature for 12 hours, the solution was treated with sodium hydrogen carbonate (30 mg, 0.36 mmol) and bromoacetonitrile (18 μL, 0.27 mmol). The resulting mixture was stirred at ambient temperature for 7 hours, before water (5 mL) and hydrochloric acid (2N aqueous solution, 50 μL) were added and then extracted with ethyl acetate (3×15 mL). The combined organic fractions were washed with water (50 mL), dried (magnesium sulphate) and concentrated in vacuo to afford the title compound as an oil, 402 mg, quantitative yield.

¹H NMR (400 MHz, CDCl₃) δ: 1.13 (s, 3H), 1.51-1.84 (m, 3H), 1.62 (s, 3H), 1.91-2.07 (m, 3H), 2.32 (s, 3H), 2.25-2.32 (m, 1H), 2.39-2.72 (m, 4H), 3.01-3.08 (m, 1H), 4.48-4.52 (m, 1H), 4.63-4.67 (m, 1H), 4.90-4.94 (m, 1H), 6.16 (s, 1H), 6.36-6.39 (m, 1H), 6.97-7.01 (m, 2H), 7.02-7.07 (m, 2H), 7.09-7.13 (m, 2H), 7.22-7.25 (m, 1H), 7.88-7.92 (m, 2H) ppm.

LRMS (ESI) 658 [M+H]⁺

### Preparations 49-52

The following compounds of the general formula shown below were prepared by a method similar to that described for Preparation 48 using the appropriate starting material and bromoacetonitrile in the presence of sodium hydrogen carbonate. The reactions were monitored by TLC or LCMS analysis. When stated, purification was undertaken by flash column chromatography on silica gel.

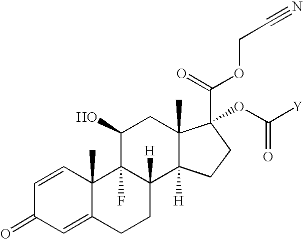

| | | | Yield | |
|---|---|---|---|---|
| No | Y | Name and NMR | (purified) | LRMS |
| 49 | 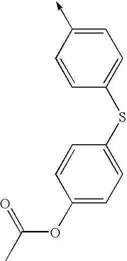 | cyanomethyl(11beta, 17alpha)-17-({4-[(4- acetoxyphenyl)thio]-benzyloxy}oxy)-9-fluoro- 11-hydroxy-3-oxoandrosta-1,4-diene-17- carboxylate Starting material: the compound as obtained in preparation 59 ¹H NMR (400 MHz, CDCl₃) δ: 1.12(s, 3H), 1.58(s, 3H), 1.51-1.65(m, 2H), 1.68-1.72 (m, 1H), 1.75-1.83(m, 1H), 1.90-2.03(m, 2H), 2.23-2.31(m, 1H), 2.33(s, 3H), 2.39- 2.58(m, 3H), 2.62-2.71(m, 1H), 3.00-3.08 (m, 1H), 4.48-4.52(m, 1H), 4.64(d, 1H), 4.91(d, 1H), 6.16(m, 1H), 6.37-6.40(m, 1H), 7.14-7.17(m, 4H), 7.21(d, 1H), 7.49- 7.52(m, 2H), 7.76-7.80(m, 2H) ppm. | 75% (yes) | (ESI) m/z 674 [M + H]⁺ |
| 50 | 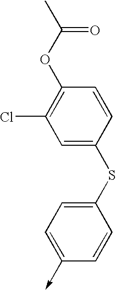 | cyanomethyl(11beta, 17alpha)-17-({4-[(4- acetoxy-3-chlorophenyl)thio]-benzoyl}oxy)- 9-fluoro-11-hydroxy-3-oxoandrosta-1,4- diene-17-carboxylate Starting material: the compound as obtained in preparation 62 ¹H NMR (400 MHz, CDCl₃) δ: 1.12(s, 3H), 1.47-1.67(m, 2H), 1.58(s, 3H), 1.72-1.83 (m, 2H), 1.89-2.04(m, 2H), 2.20-2.31(m, 1H), 2.36(s, 3H), 2.36-2.56(m, 3H), 2.62- 2.70(m, 1H), 2.99-3.07(m, 1H), 4.47-4.50 (m, 1H), 4.64(d, 1H), 4.88(d, 1H), 6.14(m, 1H), 6.35-6.38(m, 1H), 7.16(d, 1H), 7.21- 7.24(m, 2H), 7.26(d, 1H), 7.35-7.37(m, 1H), 7.52-7.53(m, 1H), 7.80-7.83(m, 2H) ppm. | 80% (no) | (ESI) m/z 708 [M + H]⁺ |
| 51 | 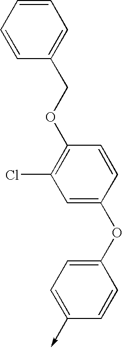 | cyanomethyl(11beta, 17alpha)-17-({4-[(4- (benzyloxy)-3-chlorophenoxy]benzoyl}oxy)- 9-fluoro-11-hydroxy-3-oxoandrosta-1,4- diene-17-carboxylate Starting material: the compound as obtained in preparation 60 ¹H NMR (400 MHz, CDCl₃) δ: 1.13(s, 3H), 1.48-1.73(m, 3H), 1.59(s, 3H), 1.76-1.84 (m, 1H), 1.91-2.07(m, 2H), 2.25-2.33(m, 1H), 2.40-2.71(m, 4H), 3.01-3.08(m, 1H), 4.49-4.53(m, 1H), 4.65(d, 1H), 4.92(d, 1H), 5.16(s, 2H), 6.16(m, 1H), 6.39(dd, 1H), 6.88-6.91(m, 1H), 6.93-6.99(m, 3H), 7.12(d, 1H), 7.22(d, 1H), 7.33-7.37(m, 1H), 7.39-7.43(m, 2H), 7.46-7.49(m, 2H), 7.87-7.91(m, 2H) ppm. | 57% (yes) | (ESI): m/z 740 [M + H]⁺ |
| 52 | 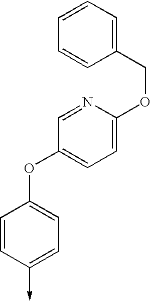 | cyanomethyl(11beta, 17alpha)-17-[(4-{[6- (benzyloxy)pyridin-3-yl]oxy}benzoyl)oxy]-9- fluoro-11-hydroxy-3-oxoandrosta-1,4-diene- 17-carboxylate Starting material: the compound as obtained in preparation 61 ¹H NMR (400 MHz, CDCl₃) δ: 1.13(s, 3H), 1.59(s, 3H), 1.49-2.07(m, 6H), 2.25-2.33 (m, 1H), 2.40-2.57(m, 3H), 2.63-2.71(m, 1H), 3.01-3.08(m, 1h), 4.49-4.51(m, 1H), 4.66(d, 1H), 4.88-4.92(m, 1H), 5.37(s, 2H), 6.16(m, 1H), 6.37-6.40(m, 1H), 6.85 (d, 1H), 6.93-6.97(m, 2H), 7.22-7.25(m, 1H), 7.32-7.42(m, 4H), 7.46-7.48(m, 2H), 7.88-7.92(m, 2H), 7.98(d, 1H) ppm. | 55% (no) | (ESI): m/z 707 [M + H]⁺ |

### Preparation 53

### cyanomethyl(11beta,17alpha)-17-{[4-(3-acetoxyphenoxy)benzoyl]oxy}-9-fluoro-11-hydroxy-3-oxoandrosta-1,4-diene-17-carboxylate

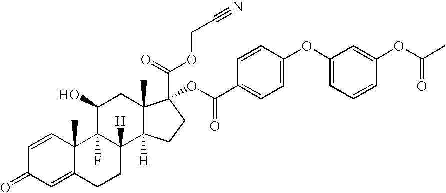

To a solution of 4-(3-acetoxyphenoxy)benzoic acid as obtained in Preparation 29 (355 mg, 1.30 mmol) in dichloromethane (5 mL) was added oxalyl chloride (250 μL, 2.90 mmol) and a drop of dimethylformamide. After stirring at ambient temperature for 3 hours the solution was concentrated in vacuo. The residue was dissolved in acetone (3 mL) and added dropwise to a cooled suspension (0° C.) of (11beta,17alpha)-9-fluoro-11,17-dihydroxy-3-oxoandrosta-1,4-diene-17-carboxylic acid [Phillipps et al, Journal of Medicinal Chemistry, 1994, pages 3717-3729] (360 mg, 0.99 mmol) in acetone (3 mL). The resulting suspension was treated with the dropwise addition of pyridine (96 μL, 1.19 mmol) and was stirred for 17 hours at ambient temperature before being diluted with ethyl acetate (50 mL). The organic extract was washed with water (2×50 mL), brine (2×50 mL), dried (sodium sulphate) and concentrated in vacuo. The residue was suspended in acetone (10 mL) and diethylamine (511 μL, 4.94 mmol) was added dropwise. The solution was left to stir at ambient temperature for 17 hours before being concentrated in vacuo. The residue was dissolved in dimethylformamide (2 mL) and treated with sodium hydrogen carbonate (300 mg, 3.57 mmol) and dropwise addition of bromoacetonitrile (354 μL, 5.08 mmol). After stirring at ambient temperature for 20 hours the solution was diluted with ethyl acetate (50 mL) and saturated sodium hydrogen carbonate solution (50 mL, aqueous). The organic fraction was washed with water (50 mL), brine (50 mL), dried (sodium sulphate) and concentrated in vacuo. The residue was purified by flash column chromatography on silica gel eluting with heptane:ethyl acetate (3:1, by volume) to afford the title compound as a white solid, 440 mg, 68% yield.

¹H NMR (400 MHz, CDCl₃) δ: 1.13 (s, 3H), 1.59 (s, 3H), 1.49-1.84 (m, 4H), 1.89-2.07 (m, 2H), 2.28 (s, 3H), 2.25-2.33 (m, 1H), 2.40-2.71 (m, 4H), 3.01-3.08 (m, 1H), 4.49-4.52 (m, 1H), 4.65 (d, 1H), 4.92 (d, 1H), 6.16 (m, 1H), 6.38 (dd, 1H), 6.81-6.82 (m, 1H), 6.90-6.95 (m, 2H), 7.00-7.03 (m, 2H), 7.23 (d, 1H), 7.35-7.39 (m, 1H), 7.89-7.93 (m, 2H) ppm.

LRMS (ESI) 658 [M+H]⁺

### Preparations 54-55

The following compounds of the general formula shown below were prepared by a method similar to that described for Preparation 53 using the appropriate starting material and (11beta,17alpha)-9-fluoro-11,17-dihydroxy-3-oxoandrosta-1,4-diene-17-carboxylic acid [Phillipps et al, Journal of Medicinal Chemistry, 1994, pages 3717-3729] followed by reaction with bromoacetonitrile. The reactions were monitored by TLC or LCMS analysis.

Acid chlorides were prepared from the corresponding carboxylic acid precursors by treatment with oxalyl chloride in dichloromethane in the presence of a catalytic amount of dimethylformamide and were used without isolation or purification.

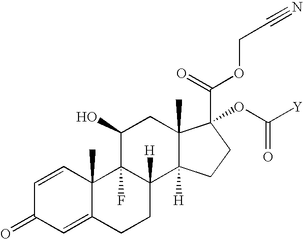

| No | Y | Name and NMR | Yield | LRMS |
|---|---|---|---|---|
| 54 | 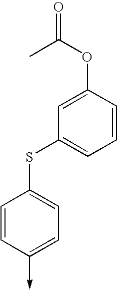 | cyanomethyl (11beta,17alpha)-17-({4-[(3- acetoxyphenyl)thio]-benzoyl}oxy)-9-fluoro- 11-hydroxy-3-oxoandrosta-1,4-diene-17- carboxylate Starting material: the compound as obtained in preparation 37. ¹H NMR (400 MHz, CDCl₃) δ: 1.12 (s, 3H), 1.48-1.85 (m, 4H), 1.59 (s, 3H), 1.90-2.05 (m, 2H), 2.23-2.32 (m, 1H), 2.30 (s, 3H), 2.39-2.72 (m, 4H), 3.01-3.08 (m, 1H), 4.48-4.53 (m, 1H), 4.62-4.66 (d, 1H), 4.90-4.94 (d, 1H), 6.16 (m, 1H), 6.39-6.40 (m, 1H), 7.12-7.15 (m, 1H), 7.21-7.27 (m, 4H), 7.32-7.35 (m, 1H), 7.39-7.43 (m, 1H), 7.79-7.81 (m, 2H) ppm. | 28% | (ESI): m/z 674 [M + H]⁺ |
| 55 | 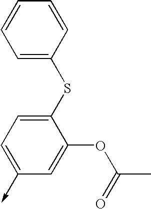 | cyanomethyl (11beta,17alpha)-17-{[3- acetoxy-4-(phenylthio)benzoyl]-oxy}-9- fluoro-11-hydroxy-3-oxoandrosta-1,4-diene- 17-carboxylate Starting material: the compound as obtained in preparation 38. ¹H NMR (400 MHz, CDCl₃) δ: 1.10 (s, 3H), 1.57 (s, 3H), 1.46-1.66 (m, 1H), 1.69-1.80 (m, 2H), 1.84-2.02 (m, 2H), 2.17-2.26 (m, 1H), 2.33 (s, 3H), 2.30-2.55 (m, 3H), 2.61-2.70 (m, 1H), 2.98-3.05 (m, 1H), 4.45-4.48 (m, 1H), 4.62 (d, 1H), 4.87 (d, 1H), 6.15 (s, 1H), 6.35-6.38 (m, 1H), 6.92 (d, 1H), 7.22-7.25 (m, 1H), 7.37-7.60 (m, 7H) ppm. | 17% | (ESI): m/z 674 [M + H]⁺ |

### Preparation 56

### (11beta,17alpha)-9-fluoro-11-hydroxy-3-oxo-17-{[4-(phenylthio)benzoyl]oxy}androsta-1,4-diene-17-carbothioic S-acid

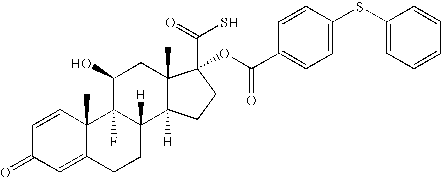

A solution of 4-(phenylthio)benzoic acid (605 mg, 2.63 mmol) in dimethylformamide (4 mL) was treated with ethyldiisopropylamine (1.12 mL, 6.40 mmol) followed by o-(7-azabenzotriazol-1-yl)-N,N,N′,N′-tetramethyluronium hexafluorophosphate (1.12 g, 2.94 mmol). After stirring at ambient temperature for 10 minutes the resulting solution was treated with (11beta,17alpha)-9-fluoro-11,17-dihydroxy-3-oxoandrosta-1,4-diene-17-carbothioic S-acid as obtained in Preparation 3 (1.00 g, 2.63 mmol). After stirring at ambient temperature for 18 hours the resulting solution was diluted with hydrochloric acid (2N aqueous solution, 20 mL) and extracted with ethyl acetate (2×20 mL). The combined organic extracts were washed with hydrochloric acid (2N aqueous solution, 10 mL), brine (10 mL), dried (magnesium sulphate) and concentrated in vacuo. The residue was purified by flash column chromatography on silica gel eluting with heptane:ethyl acetate:methanol:acetic acid (1:0:0:0 to 100:90:10:1, by volume, gradient elution) to give the title compound as a white solid, 300 mg, 19% yield.

¹H NMR (400 MHz, DMSO-d₆) δ: 1.02 (s, 3H), 1.36-1.48 (m, 2H), 1.54 (s, 3H), 1.63-1.72 (m, 1H), 1.83-1.90 (m, 1H), 1.95-2.11 (m, 3H), 2.29-2.38 (m, 2H), 2.43-2.58 (m, 1H), 2.62-2.71 (m, 1H), 2.89-2.96 (m, 1H), 4.28-4.33 (m, 1H), 5.48-5.49 (m, 1H), 6.04 (m, 1H), 6.27 (dd, 1H), 7.28-2.36 (m, 3H), 7.44-7.53 (m, 5H), 7.79-7.82 (m, 2H) ppm.

LRMS (ESI): m/z 593 [M+H]⁺

### Preparation 57

### (11beta,17alpha)-17-{[4-(3-chloro-4-hydroxyphenoxy)benzoyl]oxy}-9-fluoro-11-hydroxy-3-oxoandrosta-1,4-diene-17-carbothioic S-acid

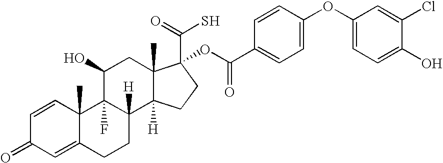

A solution of 4-(4-acetoxy-3-chlorophenoxy)benzoic acid as obtained in Preparation 33 (330 mg, 0.97 mmol) in dimethylformamide (6 mL) was cooled to 0° C. and treated with o-(7-azabenzotriazol-1-yl)-N,N,N′,N′-tetramethyluronium hexafluorophosphate (388 mg, 1.02 mmol) and ethyldiisopropylamine (372 μL, 2.14 mmol). The suspension was warmed to ambient temperature over 1 hour and treated with a solution of (11beta,17alpha)-9-fluoro-11,17-dihydroxy-3-oxoandrosta-1,4-diene-17-carbothioic S-acid as obtained in Preparation 3 (370 mg, 0.97 mmol) in N,N-dimethylformamide (6 mL). After stirring at ambient temperature for 18 hours, brine (15 mL) and ethyl acetate (20 mL) were added. The aqueous phase was acidified to pH 4 by the addition of hydrochloric acid (2N aqueous solution) and extracted with ethyl acetate (2×20 mL). The combined organic extracts were dried (sodium sulphate) and concentrated in vacuo by azeotroping with xylene. The residue was purified by flash column chromatography on silica gel eluting with heptane:ethyl acetate:dichloromethane:acetic acid (40:50:10:1, by volume) changing to dichloromethane:ethyl acetate (1:1, by volume) and then ethyl acetate:propan-2-ol:acetic acid (1:0:0 to 85:10:5, by volume, gradient elution). The residue was dissolved in methanol (7.80 mL) and water (1.75 mL) and treated with sodium hydrogen carbonate (70 mg, 0.83 mmol). After stirring at ambient temperature for 4 hours the resulting solution was treated with sodium hydrogen carbonate (10 mg, 0.12 mmol) and stirred at ambient temperature for 3 hours. The reaction mixture was neutralised to pH 7 by the addition of hydrochloric acid (0.2N aqueous solution), dried (magnesium and sodium sulphate) and concentrated in vacuo. The residue was purified by flash column chromatography on silica gel eluting with heptane:dichloromethane:ethyl acetate:acetic acid (80:20:100:1, by volume) to give the title compound as a solid, 62 mg, 20% yield.

¹H NMR (400 MHz, MeOD-d₄) δ: 1.15 (s, 3H), 1.65 (s, 3H), 1.51-1.65 (m, 2H), 1.73-1.81 (m, 1H), 1.95-2.13 (m, 3H), 2.20-2.37 (m, 1H), 2.42-2.69 (m, 3H), 2.75-2.83 (m, 1H), 2.96-3.04 (m, 1H), 4.42-4.46 (m, 1H), 6.14 (m, 1H), 6.36 (dd, 1H), 6.89-6.92 (m, 1H), 6.97-7.02 (m, 3H), 7.09 (d, 1H), 7.46 (d, 1H), 7.94-7.98 (m, 2H) ppm.

LRMS (ESI): m/z 625 [M−H]⁻

### Preparation 58

### (11beta,17alpha)-9-fluoro-11-hydroxy-3-oxo-17-[(4-phenoxybenzoyl)oxy]androsta-1,4-diene-17-carbothioic-S-acid

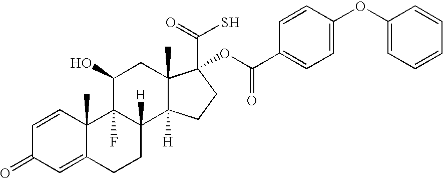

A solution of 4-phenoxybenzoic acid (352 mg, 1.61 mmol) in N,N-dimethylacetamide (8 mL, anhydrous) was treated with 4-methylmorpholine (271 μL, 2.41 mmol) followed by 1,1′-carbonylbis(1H-imidazole) (391 mg, 2.41 mmol). After stirring under nitrogen at ambient temperature for 2.5 hours further 4-methylmorpholine (271 μL, 2.41 mmol) and 1,1′-carbonylbis(1H-imidazole) (391 mg, 2.41 mmol) was added. After stirring under nitrogen at ambient temperature for 2 hours the resulting solution was treated with (11beta,17alpha)-9-fluoro-11,17-dihydroxy-3-oxoandrosta-1,4-diene-17-carbothioic S-acid as obtained in Preparation 3 (612 mg, 1.61 mmol). After stirring at ambient temperature for 5 days the resulting solution was diluted with hydrochloric acid (0.5N aqueous solution, 25 mL) and extracted with ethyl acetate (3×20 mL). The combined organic extracts were washed with water (20 mL), brine (20 mL), dried (sodium sulphate) and concentrated in vacuo. The residue was purified by flash column chromatography on silica gel eluting with (methanol:ethyl acetate:acetic acid 20:80:1):hexane (1:9 changing to 7:3, by volume) to give the title compound as a white solid, 477 mg, 52% yield.

¹H NMR (400 MHz, DMSO-d₆) δ: 1.02 (s, 3H), 1.33-1.57 (m, 2H), 1.54 (s, 3H), 1.62-1.71 (m, 1H), 1.83-1.93 (m, 1H), 1.96-2.06 (m, 2H), 2.07-2.16 (m, 1H), 2.30-2.39 (m, 2H), 2.42-2.58 (m, 1H), 2.62-2.72 (m, 1H), 2.90-2.99 (m, 1H), 4.28-4.34 (m, 1H), 5.46-5.48 (m, 1H), 6.04 (m, 1H), 6.26 (dd, 1H), 7.09-7.13 (m, 4H), 7.23-7.27 (m, 1H), 7.32-7.35 (m, 1H), 7.43-7.49 (m, 2H), 7.88-7.92 (m, 2H) ppm.

LRMS (ESI): m/z 577 [M+H]⁺575 [M−H]⁻

### Preparation 59

### (11beta,17alpha)-17-({4-[(4-acetoxyphenyl)thio]benzoyl}oxy)-9-fluoro-11-hydroxy-3-oxoandrosta-1,4-diene-17-carboxylic acid

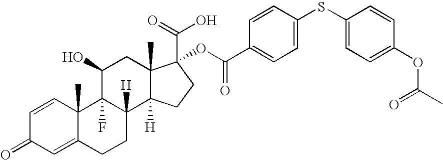

To an ice cooled solution of 4-[(4-acetoxyphenyl)thio]benzoic acid as obtained in Preparation 30 (160 mg, 698 μmol) in N,N-dimethylformamide (5 mL) was added N-ethyl-N-isopropylpropan-2-amine (292 μL, 1.68 mmol) followed by o-(7-azabenzotriazol-1-yl)-N,N,N′,N′-tetramethyluronium hexafluorophosphate (292 mg, 768 μmol). After stirring under nitrogen at ambient temperature for 1 hour the resulting solution was treated with (11beta, 17alpha)-9-fluoro-11,17-dihydroxy-3-oxoandrosta-1,4-diene-17-carboxylic acid [Phillipps et al, Journal of Medicinal Chemistry, 1994, pages 3717-3729] (254 mg, 698 μmol). After stirring at ambient temperature for 18 hours the solution was diluted with water (50 mL) and extracted with ethyl acetate (50 mL). The aqueous phase was extracted with ethyl acetate (3×50 mL). The combined organic extracts were washed with brine (3×100 mL), dried (magnesium sulphate) and concentrated in vacuo. The residue was purified by flash column chromatography on silica gel eluting with dichloromethane:ethanol:acetic acid (150:8:1, by volume) to give the title compound as a colourless solid, 153 mg, 35% yield.

¹H NMR (400 MHz, CDCl₃) δ: 1.12 (s, 3H), 1.41-1.79 (m, 4H), 1.57 (s, 3H), 1.89-2.53 (m, 6H), 2.32 (s, 3H), 2.61-2.70 (m, 1H), 2.86-3.00 (m, 1H), 4.43-4.46 (m, 1H), 6.15 (m, 1H), 6.37-6.40 (m, 1H), 7.12-7.28 (m, 5H), 7.47-7.50 (m, 2H), 7.76-7.78 (m, 2H) ppm.

LRMS (ESI): m/z 635 [M+H]⁺

### Preparations 60-61

The following compounds of the general formula shown below were prepared by a method similar to that described for Preparation 59 using the appropriate starting material in the presence of N-ethyl-N-isopropylpropan-2-amine, o-(7-azabenzotriazol-1-yl)-N,N,N′,N′-tetramethyluronium hexafluorophosphate and (11beta,17alpha)-9-fluoro-11,17-dihydroxy-3-oxoandrosta-1,4-diene-17-carboxylic acid [Phillipps et al. Journal of Medicinal Chemistry, 1994, pages 3717-3729]. The reactions were monitored by TLC or LCMS analysis.

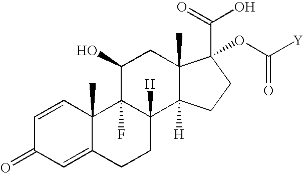

| No | R | Name and NMR | Yield | LRMS |
|---|---|---|---|---|
| 60 | 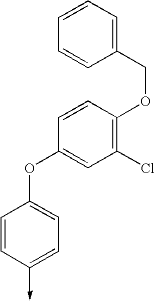 | (11beta,17alpha)-17-({4-[4-(benzyloxy)-3- chlorophenoxy]benzoyl}oxy)-9-fluoro-11- hydroxy-3-oxoandrosta-1,4-diene-17- carboxylic acid Starting material: the compound as obtained in preparation 15. ¹H NMR (400 MHz, CDCl₃) δ: 1.15 (s, 3H), 1.58 (s, 3H), 1.46-1.68 (m, 3H), 1.73-1.82 (m, 1H), 1.90-2.07 (m, 2H), 2.23-2.31 (m, 1H), 2.39-2.58 (m, 3H), 2.62-2.72 (m, 1H), 2.97-3.07 (m, 1H), 4.46-4.49 (m, 1H), 5.15 (s, 2H), 6.16 (m, 1H), 6.37-6.40 (m, 1H), 6.87-6.90 (m, 1H), 6.91-6.94 (m, 2H), 6.96 (d, 1H), 7.11 (d, 1H), 7.26 (d, 1H), 7.32-7.36 (m, 1H), 7.38-7.42 (m, 2H), 7.46-7.48 (m, 2H), 7.89-7.93 (m, 2H) ppm. | 88% | (ESI): m/z 699 [M − H]⁻ m/z 701 [M + H]⁺ |
| 61 | 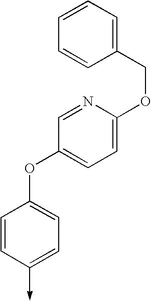 | (11beta,17alpha)-17-[(4-{[6- (benzyloxy)pyridin-3-yl]oxy}benzoyl)-oxy]-9- fluoro-11-hydroxy-3-oxoandrosta-1,4-diene- 17-carboxylic acid Starting material: the compound as obtained in preparation 22. ¹H NMR (400 MHz, CDCl₃) δ: 1.14 (s, 3H), 1.58 (s, 3H), 1.40-1.66 (m, 2H), 1.71-1.83 (m, 2H), 1.90-1.94 (m, 1H), 2.00-2.08 (m, 1H), 2.21-2.29 (m, 1H), 2.38-2.54 (m, 3H), 2.61-2.71 (m, 1H), 2.96-3.06 (m, 1H), 4.42-4.45 (m, 1H), 5.36 (s, 2H), 6.15 (m, 1H), 6.35-6.38 (m, 1H), 6.82-6.84 (m, 1H), 6.90-6.94 (m, 2H), 7.25-7.27 (m, 1H), 7.30-7.41 (m, 4H), 7.45-7.47 (m, 2H), 7.89-7.92 (m, 2H), 7.97-7.98 (m, 1H) ppm. | 70% | (ESI): m/z 668 [M + H]⁺ |

### Preparation 62

### (11beta,17alpha)-17-({4-[(4-acetoxy-3-chlorophenyl)thio]benzoyl}oxy)-9-fluoro-11-hydroxy-3-oxoandrosta-1,4-diene-17-carboxylic acid

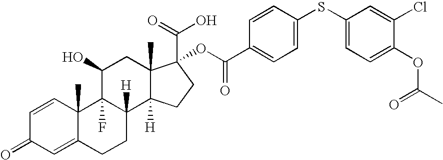

2-Chloro-4-{[4-(chlorocarbonyl)phenyl]thio}phenyl acetate was prepared from 4-[(4-acetoxy-3-chlorophenyl)thio]benzoic acid (obtained in preparation 39) following the method of Preparation 5 by treatment with oxalyl chloride in dichloromethane in the presence of a catalytic amount of N,N-dimethylformamide followed by concentration in vacuo and was used without isolation or purification. A suspension of (11beta,17alpha)-9-fluoro-11,17-dihydroxy-3-oxoandrosta-1,4-diene-17-carboxylic acid [Phillipps et al, Journal of Medicinal Chemistry, 1994, pages 3717-3729] (335 mg, 919 μmol) in acetone (6 mL) was cooled in ice and treated with pyridine (140 μL, 1.70 mmol) followed by the dropwise addition of 2-chloro-4-{[4-(chlorocarbonyl)phenyl]thio}phenyl acetate (528 mg, 1.40 mmol) in dichloromethane (4 mL). The resulting solution was allowed to warm to ambient temperature and stirred for 18 hours. The reaction mixture was cooled over ice and N-ethylethanamine (285 μL, 2.76 mmol) was added. After warming to ambient temperature the reaction mixture was stirred for 1 hour before being diluted with ethyl acetate (20 mL) and water (20 mL) and acidified to pH 4 with hydrochloric acid (2N aqueous solution). The aqueous phase was extracted with ethyl acetate (2×40 mL) and the combined organic extracts were washed with brine (50 mL), dried (sodium sulphate) and concentrated in vacuo. The residue was purified by flash column chromatography on silica gel eluting with heptane:dichloromethane:ethyl acetate:acetic acid (80:20:100:1 to 60:20:100:1, by volume, gradient elution) to give the title compound as a white solid, 225 mg, 37% yield.

¹H NMR (400 MHz, CDCl₃) δ: 1.16 (s, 3H), 1.58 (s, 3H), 1.47-1.69 (m, 2H), 1.74-1.82 (m, 2H), 1.89-1.96 (m, 1H), 2.02-2.11 (m, 1H), 2.21-2.30 (m, 1H), 2.37 (s, 3H), 2.37-2.57 (m, 3H), 2.61-2.72 (m, 1H), 2.97-3.05 (m, 1H), 4.47-4.50 (m, 1H), 6.16 (br s, 1H), 6.39 (dd, 1H), 7.16 (d, 1H), 7.22-7.24 (m, 3H), 7.36 (dd, 1H), 7.53 (d, 1H), 7.83-7.85 (m, 2H) ppm.

LRMS (ESI): m/z 669 [M+H]⁺

### Preparation 63

### cyanomethyl(11beta,17alpha)-17-{[3-(benzyloxy)-4-phenoxybenzoyl]oxy}-9-fluoro-11-hydroxy-3-oxoandrosta-1,4-diene-17-carboxylate

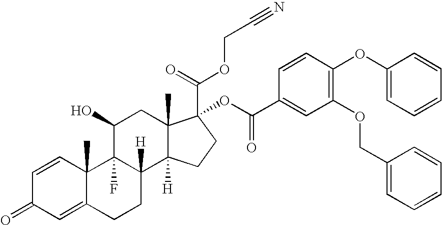

To an ice cooled solution of 3-(benzyloxy)-4-phenoxybenzoic acid as obtained in Preparation 46 (313 mg, 977 μmol) in N,N-dimethylformamide (5 mL) was added o-(7-azabenzotriazol-1-yl)-N,N,N′,N′-tetramethyluronium hexafluorophosphate (409 mg, 1.08 mmol) followed by N-ethyl-N-isopropylpropan-2-amine (408 μL, 2.34 mmol). After stirring at ambient temperature for 1 hour a solution of (11beta,17alpha)-9-fluoro-11,17-dihydroxy-3-oxoandrosta-1,4-diene-17-carboxylic acid [Phillipps et al, Journal of Medicinal Chemistry, 1994, pages 3717-3729] (356 mg, 977 μmol) in dimethylformamide (3 mL) was added over a period of 5 minutes. After stirring at ambient temperature for 18 hours the resulting solution was diluted with water (50 mL) and ethyl acetate (50 mL). The aqueous phase was extracted with ethyl acetate (3×50 mL). The combined organic extracts were washed with water (3×150 mL), dried (magnesium sulphate) and concentrated in vacuo. The residue was dissolved in dimethylformamide (4 mL) and cooled over ice before the addition of bromoacetonitrile (80 μL, 1.20 mmol) and sodium hydrogen carbonate (132 mg, 1.58 mmol). The reaction mixture was warmed to ambient temperature and stirred under nitrogen for 18 hours before being diluted with water (20 mL) and ethyl acetate (20 mL). The aqueous phase was extracted with ethyl acetate (3×20 mL) and the combined organic extracts were washed with brine (3×80 mL), dried (magnesium sulphate) and concentrated in vacuo. The residue was purified by flash column chromatography on silica gel eluting with ethyl acetate:heptane (1:1, by volume) to give the title compound as a colourless solid, 351 mg, 51% yield.

¹H NMR (400 MHz, CDCl₃) δ: 1.13 (s, 3H), 1.59 (s, 3H), 1.50-1.86 (m, 4H), 1.92-2.07 (m, 2H), 2.27-2.35 (m, 1H), 2.40-2.59 (m, 3H), 2.64-2.72 (m, 1H), 3.00-3.07 (m, 1H), 4.50-4.52 (m, 1H), 4.65 (d, 1H), 4.92 (d, 1H), 5.11 (s, 2H), 6.16 (m, 1H), 6.38 (dd, 1H), 6.96 (d, 1H), 6.98-7.01 (m, 2H), 7.12-7.38 (m, 9H), 7.52-7.54 (m, 1H), 7.64 (m, 1H) ppm.

LRMS (ESI): m/z 706 [M+H]⁺

### Preparations 64-65

The following compounds of the general formula shown below were prepared by a method similar to that described for Preparation 63 using the appropriate starting material and (11beta,17alpha)-9-fluoro-11,17-dihydroxy-3-oxoandrosta-1,4-diene-17-carboxylic acid [Phillipps et al, Journal of Medicinal Chemistry, 1994, pages 3717-3729] followed by reaction with bromoacetonitrile. The reactions were monitored by TLC or LCMS analysis.

Activated acids were prepared from the corresponding carboxylic acid precursors by treatment with N-ethyl-N-isopropylpropan-2-amine and o-(7-azabenzotriazol-1-yl)-N,N,N′,N′-tetramethyluronium hexafluorophosphate in dimethylformamide and were used without isolation or purification.

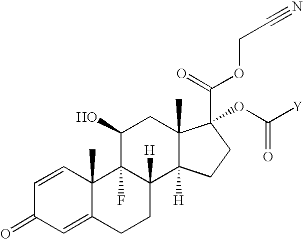

| No | Y | Name and NMR | Yield | LRMS |
|---|---|---|---|---|
| 64 | 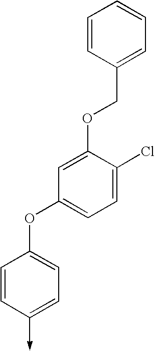 | cyanomethyl (11beta,17alpha)-17-({4-[3- (benzyloxy)-4- chlorophenoxy]benzoyl}oxy)-9-fluoro-11- hydroxy-3-oxoandrosta-1,4-diene-17- carboxylate Starting material: the compound as obtained in preparation 13. ¹H NMR (400 MHz, CDCl₃) δ: 1.13 (s, 3H), 1.59 (s, 3H), 1.50-1.85 (m, 4H), 1.92-2.08 (m, 2H), 2.26-2.34 (m, 1H), 2.40-2.61 (m, 3H), 2.68-2.71 (m, 1H), 3.02-3.09 (m, 1H), 4.50-4.53 (m, 1H), 4.67 (d, 1H), 4.91 (d, 1H), 5.11 (s, 2H), 6.16 (m, 1H), 6.37-6.40 (m, 1H), 6.58 (dd, 1H), 6.68 (d, 1H), 6.90-6.94 (m, 2H), 7.23 (d, 1H), 7.31-7.42 (m, 6H), 7.87-7.90 (m, 2H) ppm. | 49% | (ESI): m/z 738 [M − H]⁻ 740 [M + H]⁺ |
| 65 | 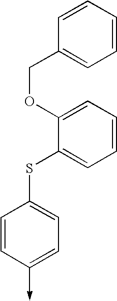 | cyanomethyl (11beta,17alpha)-17-[(4-{[2- (benzyloxy)phenyl]lthio}benzoyl)oxy]-9- fluoro-11-hydroxy-3-oxoandrosta-1,4- diene-17-carboxylate Starting material: the compound as obtained in preparation 14. ¹H NMR (400 MHz, CDCl₃) δ: 1.12 (s, 3H), 1.59 (s, 3H), 1.48-1.83 (m, 4H), 1.91-2.05 (m, 2H), 2.23-2.31 (m, 1H), 2.39-2.56 (m, 3H), 2.62-2.71 (m, 1H), 3.00-3.07 (m, 1H), 4.48-4.50 (m, 1H), 4.62 (d, 1H), 4.90 (d, 1H), 5.09 (s, 2H), 6.16 (m, 1H), 6.37-6.40 (m, 1H), 6.97-7.02 (m, 2H), 7.13-7.18 (m, 4H), 7.22-7.28 (m, 4H), 7.36-7.40 (m, 1H), 7.48-7.50 (m, 1H), 7.73-7.77 (m, 2H) ppm. | Quantitative (no purify- cation) | |

### Preparation 66

### 1-(benzyloxy)-4-(trimethoxymethyl)benzene

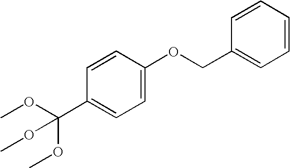

A solution of 4-(trimethoxymethyl)phenol [Ramig K, Englander M, Kallashi F, Livchits L, Zhou J, Tetrahedron Letters, 2002, pages 7731-7734] (950 mg, 4.79 mmol) in dimethylformamide (10 mL) was treated with cesium carbonate (4.69 g, 14.40 mmol) followed by (bromomethyl)benzene (570 μL, 4.79 mmol). After stirring at ambient temperature for 2 hours the resulting suspension was diluted with saturated sodium hydrogen carbonate solution (50 mL, aqueous) and extracted with ethyl acetate (2×50 mL). The combined organic extracts were washed with brine (50 mL), dried (magnesium sulphate) and concentrated in vacuo. The residue was purified by flash column chromatography on silica gel eluting with heptane:ethyl acetate (9:1 to 4:6, by volume, gradient elution) to give the title compound as a white solid, 512 mg, 37% yield.

¹H NMR (400 MHz, DMSO-d₆) δ: 3.03 (s, 9H), 5.13 (s, 2H), 7.04-7.08 (m, 2H), 7.34-7.44 (m, 5H), 7.47-7.50 (m, 2H) ppm.

LRMS (ESI): m/z 257 [M-OCH₃]⁺

### Preparation 67

### 3-chloro-4-hydroxyphenyl thiocyanate

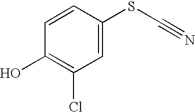

A solution of bromine (900 μL, 18.00 mmol) in acetic acid (7 mL) was added dropwise over 30 minutes to a suspension of 2-chlorophenol (1.80 mL, 17.40 mmol) and sodium thiocyanate (5.00 g, 62.00 mmol) in acetic acid (7 mL) cooled in a water bath (to 23° C.). The reaction was stirred at ambient temperature for 1 hour 45 minutes before the addition of water (75 mL) and ethyl acetate (75 mL). The resulting solid suspension was filtered through celite and washed with ethyl acetate:water (1:1, 150 mL). The organic extract was dried (magnesium sulphate), filtered through a small pad of silica and concentrated in vacuo. The residue was dissolved in methanol (100 mL) and azeotroped with cyclohexane (50 mL) before concentrating in vacuo. The residue was dissolved in tert-butyl methyl ether (100 mL) and dichloromethane was added (100 mL). The solid suspension was filtered off and filtrate concentrated in vacuo to give the title compound as a yellow solid, 2.11 g, 65% yield.

¹H NMR (400 MHz, CDCl₃) δ: 7.10 (d, 1H), 7.42 (dd, 1H), 7.60 (d, 1H) ppm.

LRMS (ESI): m/z 183 [M−H]⁻

### Preparation 68

### 2-chloro-4-mercaptophenol

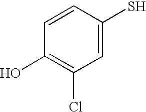

To an ice cooled solution of 3-chloro-4-hydroxyphenyl thiocyanate as obtained in Preparation 67 (2.11 g, 11.40 mmol) in tetrahydrofuran (30 mL) was added lithium aluminium hydride in tetrahydrofuran (1M, 60 mL, 60 mmol) dropwise. The reaction mixture was slowly warmed to ambient temperature over 1 hour and stirred for a further 4 hours at ambient temperature. The reaction mixture was cooled over ice, treated with the dropwise addition of water:tetrahydrofuran (1:1, 20 mL) and acidified to pH1 by the addition of hydrochloric acid (1N aqueous solution, 35 mL). The resulting solution was extracted with ethyl acetate (3×70 mL). The combined organic extracts were dried (sodium sulphate) and concentrated in vacuo to give the title compound as an orange oil, 1.61 g, quantitative yield.

¹H NMR (400 MHz, CDCl₃) δ: 6.91 (d, 1H), 7.16 (dd, 1H), 7.33 (d, 1H) ppm.

LRMS (ESI): m/z 159 [M−H]⁻

### Preparation 69

### (11beta,16alpha,17alpha)-9-fluoro-11,17-dihydroxy-16-methyl-3-oxoandrosta-1,4-diene-17-carboxylic acid

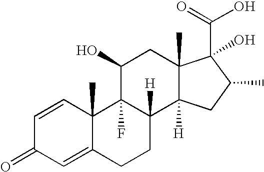

A suspension of dexamethasone (5.50 g, 14.01 mmol) in methanol (200 mL) was treated with potassium carbonate (4.30 g, 31.10 mmol). The resulting suspension was stirred at ambient temperature for 3 hours whilst bubbling through air and the suspension slowly became a pale yellow solution. The solution was stirred for a further 15 hours open to the atmosphere. It was then concentrated to low volume in vacuo and hydrochloric acid (2N aqueous solution) (50 mL) followed by water (200 mL) were slowly added. The precipitated solid was stirred at ambient temperature for 2 hours. The solid was then collected by filtration and sucked dry, affording the title compound as a white solid, 5.30 g, 99% yield.

¹H NMR (400 MHz, DMSO-d₆) δ: 0.88 (d, 3H), 1.02 (s, 3H), 1.01-1.10 (m, 2H) 1.31-1.42 (m, 1H), 1.51 (s, 3H), 1.52-1.57 (m, 1H), 1.59-1.70 (m, 1H), 1.75-1.83 (m, 1H), 1.97-2.07 (m, 2H), 2.26-2.43 (m, 2H), 2.59-2.69 (m, 1H), 2.79-2.89 (m, 1H), 4.11-4.19 (m, 1H), 5.24 (bs, 1H), 6.02 (s, 1H), 6.23 (dd, 1H), 7.31 (d, 1H), 12.30 (br s, 1H) ppm.

LRMS (ESI): m/z 377 [M−H]⁻

### Preparation 70

### (11beta,16alpha,17alpha)-9-fluoro-11,17-dihydroxy-16-methyl-3-oxoandrosta-1,4-diene-17-carbothioic S-acid

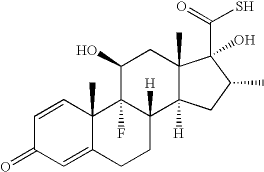

A solution of (11beta, 16alpha,17alpha)-9-fluoro-11,17-dihydroxy-16-methyl-3-oxoandrosta-1,4-diene-17-carboxylic acid as obtained in Preparation 69 (1.00 g, 2.64 mmol) in N,N-dimethylformamide (10 mL) was treated with 1,1′-carbonyl diimidazole (857 mg, 5.28 mmol). The reaction mixture was stirred at ambient temperature for 2 hours. Solid lithium sulfide was then added portionwise and after completion of the addition the reaction mixture was stirred for a further 30 minutes at ambient temperature. The reaction mixture was poured onto a mixture of ice/hydrochloric acid (2N aqueous solution) (100 mL) and the precipitated solid was collected by filtration and sucked dry, affording the title compound as a white solid, 980 mg, 91% yield.

¹H NMR (400 MHz, DMSO-d₆) δ: 0.83 (d, 3H), 0.98 (s, 3H), 1.01-1.18 (m, 2H), 1.31-1.46 (m, 1H), 1.51 (s, 3H), 1.60-1.81 (m, 3H), 1.98-2.14 (m, 2H), 2.29-2.45 (m, 2H), 2.59-2.69 (m, 1H), 2.87-2.96 (m, 1H), 4.16 (bs, 1H), 6.02 (s, 1H), 6.24 (dd, 1H), 7.31 (d, 1H) ppm.

LRMS (ESI): m/z 393 [M−H]⁻

### Preparation 71

### 4-chloro-3-methoxybenzenethiol

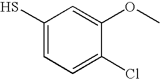

To a room temperature solution of magnesium (366 mg, 15 mmol) in anhydrous tetrahydrofuran (2 mL) under a nitrogen atmosphere was added a crystal of iodine. This was followed by the drop-wise addition of 3-bromo-5-chloroanisole (3.00 g, 13.5 mmol) in anhydrous tetrahydrofuran (9 mL) over 30 minutes. The resultant black solution was then heated to reflux for 90 minutes. The reaction mixture was cooled to room temperature and stirred at ambient temperature for 1 hour. Sulphur (360 mg, 0.83 mmol) was then added. An exotherm was noted along with a colour change from a black to a grey solution. The reaction mixture was stirred for an hour then left to stand for 16 hours (convenience). The reaction mixture was heated to reflux for an hour then cooled back to room temperature before being poured into an ice (20 g)/water (20 mL)/hydrogen chloride (37% aqueous solution) (5 mL) solution. The aqueous solution was extracted with tert-butyl methyl ether (2×50 mL), then the organic layers were combined and washed with sodium hydroxide (10% aqueous solution) (3×30 mL). The combined aqueous extracts were treated with hydrogen chloride (37% aqueous solution) (5-10 mL) and a white solid appeared. The aqueous was then re-extracted with tert-butyl methyl ether (3×50 mL), and the combined organics were dried (sodium sulphate), and evaporated in vacuo to afford the title compound as a light yellow oil, 1.19 g, 60% yield.

¹H NMR (DMSO-d₆) δ: 3.82 (s, 3H), 5.62 (br s, 1H), 6.86 (dd, 1H), 7.10 (d, 1H), 7.26 (d, 1H) ppm.

LRMS (ESI): m/z 172.79 [M{³⁵Cl}−H]⁻, 174.76 [M{³⁷Cl}−H]⁻

### Preparation 72

### 4-[(4-chloro-3-methoxyphenyl)thio]benzonitrile

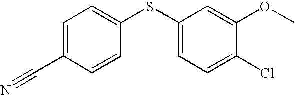

To a room temperature solution of 4-fluorobenzonitrile (820.0 mg, 6.8 mmol) in anhydrous acetonitrile (14 mL) was added cesium carbonate (4440 mg, 13.6 mmol), salox (196 mg, 1.43 mmol), copper (1) oxide (53.7 mg, 0.37 mmol) and 4-chloro-3-methoxybenzenethiol as obtained in Preparation 71 (1190 mg, 6.81 mmol and the resultant suspension was heated to reflux under a nitrogen atmosphere, for 16 hours. The reaction mixture changed colour from red to orange over time. The reaction mixture was then cooled to room temperature and a solid was noted. The reaction mixture was diluted with water (50 mL) and ethyl acetate (30 mL). Hydrogen chloride (0.2N aqueous solution) was added until the pH rendered 5-6. And the layers were separated. The aqueous was re-extracted with ethyl acetate (2×50 mL). The organic layers were combined, dried (magnesium sulphate) and evaporated in vacuo to give a white solid. This was dissolved in hot methanol (500 mL) and any in-soluble material was removed by filtration. After cooling the title compound crystallized as a white solid (685 mg). The mother liqueurs were evaporated in vacuo then purified by column chromatography on silica gel eluting with heptanes to heptanes:ethyl acetate 95:5 to afford the title compound as a white solid, 1.28 g in total, 93% yield.

¹H NMR (400 MHz, CDCl₃) δ: 3.90 (s, 3H), 7.05-7.08 (m, 2H), 7.18-7.21 (m, 2H), 7.42 (d, 1H), 7.50-7.54 (m, 2H) ppm.

LRMS (ESI): m/z 275.8 [M+H]⁺

### Preparation 73

### 4-[(4-chloro-3-methoxyphenyl)thio]benzoic acid

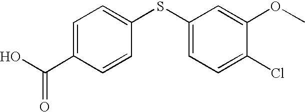

To a room temperature solution of 4-[(4-chloro-3-methoxyphenyl)thio]benzonitrile as obtained in Preparation 72, (1.28 g, 4.64 mmol) in ethanol/water (5 mL/25 mL) was added sodium hydroxide (1.80 g, 45 mmol) and the resultant solution was heated to reflux for 16 hours. The reaction mixture was cooled to room temperature then hydrochloric acid (concentrated solution) (3.8 mL) was added drop-wise to neutralize the solution giving a milky white suspension. This solution was extracted with ethyl acetate (3×200 mL). The organic layers were combined, dried (magnesium sulphate), filtered and evaporated in vacuo to afford the title compound as a yellow powder, 1.25 g, 91% yield.

¹H NMR (400 MHz, DMSO-d₆) δ: 3.85 (s, 3H), 7.03 (dd, 1H), 7.26 (d, 1H), 7.28-7.32 (m, 2H), 7.51 (d, 1H), 7.86-7.89 (m, 2H), 12.96 (br s, 1H) ppm.

LRMS (ESI): m/z 293 [M{³⁵Cl}−H]⁻, 295 [M{³⁷Cl}−H]⁻

### Preparation 74

### 4-[(4-chloro-3-hydroxyphenyl)thio]benzoic acid

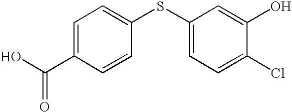

To a room temperature solution of hydrogen bromide (33% in acetic acid solution) (100 mL) acetic acid (10 mL) and water (10 mL) was added 4-[(4-chloro-3-methoxyphenyl)thio]benzoic acid as obtained in Preparation 73, (4.00 g, 14 mmol) and the resultant solution was heated at 135° C. for 16 hours. The solution was now dark brown in colour. Heating was stopped and the reaction mixture was cooled to room temperature before evaporating the solvents in vacuo to give a brown solid. This was dissolved in methanol (10 mL) and water (100 mL) was added drop-wise to give a purple precipitate. This was filtered and dried under air to afford the title compound as a purple solid, 3.57 g, 94% yield.

¹H NMR (400 MHz, DMSO-d₆) δ: 6.91 (dd, 1H), 7.01 (d, 1H), 7.28-7.33 (m, 2H), 7.41 (d, 1H), 7.85-7.91 (m, 2H), 10.55 (br s, 1H), 12.98 (br s, 1H) ppm.

LRMS (ESI): m/z 278.73 [M{³⁵Cl}−H]⁻, 280.66 [M{³⁷Cl}−H]⁻

### Preparation 75

### 4-[(3-acetoxy-4-chlorophenyl)thio]benzoic acid

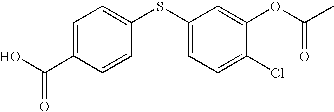

A suspension of 4-[(4-chloro-3-hydroxyphenyl)thio]benzoic acid as obtained in Preparation 74 (2.09 g, 7.45 mmol) in dichloromethane (25 mL) was cooled to 5° C. and treated with pyridine (3.01 mL, 37.20 mmol) followed by acetic anhydride (1.05 mL, 11.20 mmol) at. The dark purple solution was stirred and allowed to warm to ambient temperature over 6 hours. The solvent was removed in vacuo and the residue was partitioned between ethyl acetate (50 mL) and hydrochloric acid (2N aqueous solution) (50 mL). The aqueous layer was extracted with ethyl acetate (1×30 mL). The combined organic extracts were dried (magnesium sulphate) and the solvent removed in vacuo to afford the title compound as a purple solid, 2.32 g, 96% yield.

¹H NMR (400 MHz, DMSO-d₆) δ: 2.33 (s, 3H), 7.36-7.41 (m, 3H), 7.45 (d, 1H), 7.66 (d, 1H), 7.90-7.94 (m, 2H), 12.95 (br s, 1H) ppm.

LRMS (ESI): m/z 321 [M−H]⁻

### Preparation 76

### 4-(4-chloro-3-iodophenoxy)benzonitrile

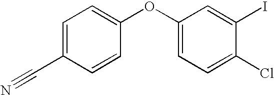

To a room temperature solution of 4-chloro-3-iodo-phenol (1.00 g, 3.93 mmol) and 4-fluorobenzonitrile (428 mg, 3.94 mmol) in anhydrous N,N-dimethylformamide (30 mL) was added cesium carbonate (1.54 g, 4.72 mmol) and the resultant solution was stirred at 80° C. under nitrogen gas for 2 hours. The reaction mixture was then cooled to room temperature and stirred at ambient temperature for 16 hours. The reaction mixture was partitioned between ethyl acetate (50 mL) and water (50 mL). The aqueous layer was extracted with ethyl acetate (2×40 mL), and the combined organics dried (magnesium sulphate), filtered and evaporated in vacuo. The crude material was purified by ISCO chromatography (80 g silica cartridge), eluting with 100% heptanes to heptanes/ethyl acetate 4:1 to afford the title compound as a white solid, 610 mg, 44% yield.

¹H NMR (400 MHz, CDCl₃) δ: 7.00-7.05 (m, 3H), 7.47 (d, 1H), 7.57 (d, 1H), 7.63-7.67 (m, 2H) ppm.

LRMS (ESI): m/z 356 [M+H]⁺

### Preparation 77

### 4-(4-chloro-3-hydroxyphenoxy)benzoic acid

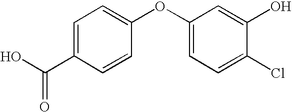

4-(4-chloro-3-iodophenoxy)benzonitrile as obtained in Preparation 76 (5.13 g, 14.4 mmol), potassium hydroxide (1.62 g, 28.9 mmol), 2-di-tert-butylphosphino-2′,4′,6′-triisopropylbiphenyl (1.23 g, 2.89 mmol) and bis(dibenzylidine acetone) palladium(0) (0.83 g, 1.44 mmol) were stirred in 1,4-dioxane/water (3:1, 40 mL) at 90° C. for 16 hours. The reaction mixture was then cooled to room temperature then diluted with water (30 mL) followed by the addition of sodium hydroxide (11.5 g, 289 mmol). The resultant solution was then heated at reflux for 16 hours. The reaction mixture was then allowed to cool to room temperature, then was filtered through celite and washed with ethyl acetate (2×500 mL). The aqueous layer was then acidified by the addition of hydrochloric acid (2N aqueous solution) (to pH1 by universal indicator paper) resulting in formation of light brown suspension. The aqueous layer was then extracted into ethyl acetate (2×300 mL), and the organic extracts were combined, dried (magnesium sulphate), filtered and evaporated in vacuo to leave a brown solid. TLC indicated a mixture of acid and primary amide. The solid was then taken up into ethanol (50 mL) and water (100 mL), followed by the addition of sodium hydroxide (11.5 g, 289 mmol) and the reaction mixture was again heated at reflux 16 hours. The reaction mixture was then allowed to cool to room temperature, then was washed with ethyl acetate (2×500 mL). The aqueous layer was then acidified by the addition of hydrochloric acid (2N aqueous solution) (to pH1 by universal indicator paper) resulting in formation of a cloudy precipitate. The aqueous layer was then extracted into ethyl acetate (2×300 mL), and the organic extracts were combined, washed with brine (1×600 mL), dried (magnesium sulphate), filtered and evaporated in vacuo to leave a brown solid. The crude solid was then purified by silica gel flash chromatography (eluent: ethyl acetate) to give a light brown solid that was then triturated from ethyl acetate/heptanes to afford the title target as an off-white microcrystalline solid, 2.80 g, 73% yield.

¹H NMR (400 MHz, DMSO-d₆) δ: 6.55 (dd, 1H), 6.65 (d, 1H), 7.05-7.10 (m, 2H), 7.37 (d, 1H), 7.91-7.98 (m, 2H), 10.48 (s, 1H), 12.85 (s, 1H) ppm.

LRMS (ESI): m/z 265 [M+H]⁺

### Preparation 78

### 4-(3-acetoxy-4-chlorophenoxy)benzoic acid

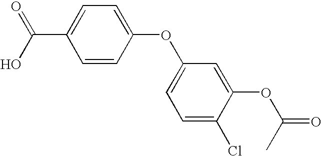

The following compound was prepared by a method similar to that described for Preparation 75 using 4-(4-chloro-3-hydroxyphenoxy)benzoic acid as obtained in Preparation 77 as starting material and acetic anhydride in the presence of pyridine to afford a beige solid, 68% yield. The reaction was monitored by TLC or LCMS analysis.

¹H NMR (400 MHz, CDCl₃) δ: 2.35 (s, 3H), 6.89-6.95 (m, 2H), 7.05-7.09 (m, 2H), 7.45 (d, 1H), 8.09-8.13 (m, 2H) ppm

LRMS (ESI): m/z 305 [M−H]⁻

### Preparation 79

### 4-[4-(methylthio)phenoxy]benzonitrile

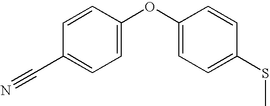

To a room temperature solution of 4-(methylthio)phenol (1.74 g, 12.4 mmol) and 4-fluorobenzonitrile (1.5 g, 12.4 mmol) in N,N-dimethylformamide (25 mL) was added cesium carbonate (4.2 g, 13 mmol) and the resultant suspension was degassed for 10 mins. The reaction was then heated to 80° C. overnight under nitrogen. The reaction mixture was acidified (pH 2/3) with drop-wise addition of hydrochloric acid (2N aqueous solution) before being poured reaction into water (20 mL) and extracting with ethyl acetate (3×20 mL). Organics were combined, washed with water (2×50 mL), dried (magnesium sulphate), filtered and concentrated in vacuo to afford the crude material as a pale orange oil, 2.98 g, 100% yield. This was taken on with no further purification.

¹H NMR (400 MHz, CDCl₃) δ: 2.50 (s, 3H), 6.97-7.02 (m, 4H), 7.30 (d, 2H), 7.59 (d, 2H) ppm.

LRMS (ESI): m/z 242 [M+H]⁺

### Preparations 80-81

The following compounds were prepared by a method similar to that described for Preparation 79 using the appropriate starting material and 4-fluorobenzonitrile in the presence of cesium carbonate. The reactions were monitored by TLC or LCMS analysis.

| No | Structure | Name and NMR | Yield | LRMS |
|---|---|---|---|---|
| 80 | 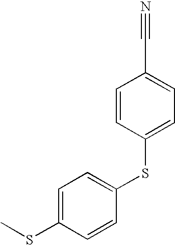 | 4-{[4-(methylthio)phenyl]thio} benzonitrile Starting material: 4- (methylmercapto)phenol | White solid 65% (purified by column chromatography on silica gel eluting with Heptanes ethyl acetate 3:1) | |
| 81 | 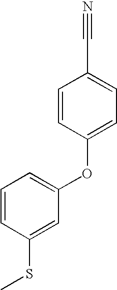 | 4-{[3-(methylthio)phenoxy] benzonitrile Starting material: 3- (methylmercapto)phenol ¹H NMR (400 MHz, DMSO-d₆) δ: 2.49 (s, 3H), 6.88 (2 × dd, 1H), 7.03 (t, 1H), 7.09-7.13 (m, 2H), 7.15 (2 × dd, 1H), 7.38 (t, 1H), 7.82-7.86 (m, 2H) ppm. | White solid 71% (purified by column chromatography on silica gel eluting with Heptanes:dichloromethane 1:1) | (ESI): m/z 242 [M + H]⁺ |

### Preparation 82

### 4-[4-(methylthio)phenoxy]benzoic acid

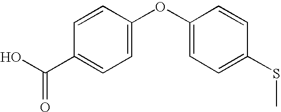

To a room temperature suspension of 4-[4-(methylthio)phenoxy]benzonitrile as obtained in Preparation 79 (2.98 g, 12.4 mmol) in ethanol (20 mL) and water (20 mL) was added sodium hydroxide (4.46 g, 111 mmol). The resulting mixture was heated to 100° C. under nitrogen for 72 hours. A white milky suspension was observed that cleared on heating. The reaction mixture was diluted with water (50 mL), acidified to pH 2/3 by addition of hydrochloric acid (2N aqueous solution). The solution was then transferred to a separating funnel and extracted with ethyl acetate (3×100 mL). The organic extracts were combined, washed with water (20 mL), then dried (magnesium sulphate), filtered and concentrated in vacuo to afford the title compound as a pale yellow solid, 3.2 g, 99% yield.

¹H NMR (400 MHz, DMSO-d₆) δ: 2.42 (s, 3H), 6.99-7.03 (m, 2H), 7.05-7.09 (m, 2H), 7.33-7.38 (m, 2H), 7.90-7.95 (m, 2H) ppm. No acid proton visible

LRMS (ESI): m/z 259 [M−H]⁻

### Preparations 83-84

The following compounds were prepared by a method similar to that described for Preparation 82 using the appropriate starting material in the presence of sodium hydroxide. The reactions were monitored by TLC or LCMS analysis.

| No | Structure | Name and NMR | Yield | LRMS |
|---|---|---|---|---|
| 83 | 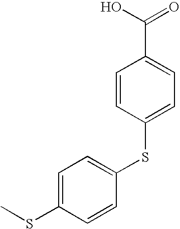 | 4-{(4-(methylthio)phenyl]thio} benzoic acid Starting material: 4-{[4- (methylthio)phenyl]thio}benzonitrile as obtained in Preparation 80. ¹H NMR (400 MHz, DMSO-d₆) δ: 2.49 (s, 3H), 7.15-7.19 (m, 2H), 7.31-7.34 (m, 2H), 7.41-7.45 (m, 2H), 7.81-7.84 (m, 2H), 12.82 (brs, 1H) ppm. | White solid 68% (Crude compound was trituated with dichloromethane) | (ESI): m/z 275 [M − H]⁻ |
| 84 | 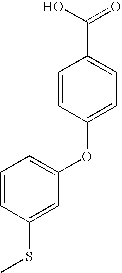 | 4-[3-(methylthio)phenoxy]benzoic acid Starting material: 4-[3- (methylthio)phenoxy]benzonitrile as obtained in Preparation 81. ¹H NMR (400 MHz, DMSO-d₆) δ: 2.48 (s, 3H), 6.85 (2 × dd, 1H), 6.99 (t, 1H), 7.02-7.06 (m, 2H), 7.11 (2 × dd, 1H), 7.37 (t, 1H), 7.93-7.96 (m, 2H), 12.83 (s, 1H) ppm. | White solid 96% (Crude compound was trituated with dichloromethane) | (ESI): m/z 261 [M + H]⁺ |

### Preparation 85

### (11beta,16alpha,17alpha)-9-fluoro-17-{[(fluoromethyl)thio}carbonyl]-11-hydroxy-16-methyl-3-oxoandrosta-1,4-dien-17-yl 4-(4-acetoxy-3-chlorophenoxy)benzoate

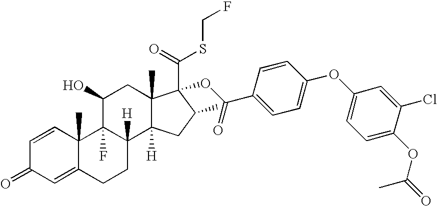

A solution of 4-(4-acetoxy-3-chlorophenoxy)benzoic acid from preparation 33 (472 mg, 1.54 mmol) in N,N-dimethylformamide (6 mL) was cooled to 5° C. and treated with N-ethyl-N-isopropylpropan-2-amine (0.59 mL, 3.41 mmol) followed by o-(7-azabenzotriazol-1-yl)-N,N,N′,N′-tetramethyluronium hexafluorophosphate (673 mg, 1.77 mmol) portion wise. The solution was stirred under a nitrogen atmosphere for 30 minutes then (11beta,16alpha,17alpha)-9-fluoro-11,17-dihydroxy-16-methyl-3-oxoandrosta-1,4-diene-17-carboxylic acid as obtained in Preparation 69 (607 mg, 1.54 mmol) was added portion wise. The reaction mixture was allowed to warm to ambient temperature and stirred for 1 hour and then treated with a solution of bromofluoromethane (33% w/v solution in 2-butanone, 2.40 mL, 3.84 mmol). The reaction mixture was stirred at ambient temperature for 15 hours then partitioned between ethyl acetate (60 mL) and hydrochloric acid (2N aqueous solution) (50 mL). The aqueous layer was extracted with ethyl acetate (2×30 mL) and the combined organic extracts dried (magnesium sulphate) and concentrated to dryness in vacuo. The residue was purified by flash column chromatography on silica gel eluting with dichloromethane:ethyl acetate (100:0 to 80:20, by volume, gradient elution). The afforded product was purified a second time by flash column chromatography on silica gel eluting with dichloromethane: ethyl acetate (100:0 to 85:15, by volume, gradient elution) to afford the title compound as a white foam, 238 mg, 22% yield.

¹H NMR (400 MHz, DMSO-d₆) δ: 0.95 (d, 3H), 1.11 (s, 3H), 1.26-1.35 (m, 1H), 1.42-1.52 (m, 1H), 1.53 (s, 3H), 1.83-2.00 (m, 3H), 2.17-2.30 (m, 2H), 2.36 (s, 3H), 2.35-2.41 (m, 1H), 2.42-2.58 (m, 1H), 2.62-2.73 (m, 1H), 3.36-3.46 (m, 1H), 4.29 (bs, 1H), 5.55 (d, 1H), 5.92 (s, 1H), 6.05 (d, 2H), 6.27 (dd, 1H), 7.18 (dd, 1H), 7.20 (dt, 2H), 7.33 (d, 1H), 7.39 (d, 1H), 7.44 (d, 1H) 7.93 (dt, 2H) ppm.

LRMS (ESI): m/z 715 [M+H]⁺

### Preparation 86

### Fluoromethyl (6alpha,11beta,16alpha,17alpha)-17-{[4-(4-acetoxy-3-chlorophenoxy)benzoyl]oxy}-6,9-difluoro-11-hydroxy-16-methyl-3-oxoandrosta-1,4-diene-17-carboxylate

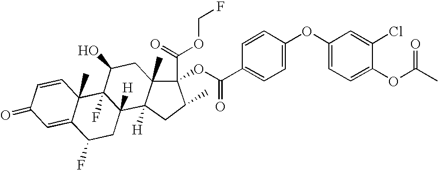

The title compound was prepared by a similar method to that described for Preparation 85 using (6alpha,11beta, 16alpha, 17alpha)-6,9-difluoro-11,17-dihydroxy-16-methyl-3-oxoandrosta-1,4-diene-17-carboxylic acid (Journal of Organic Chemistry (1986), 51(12), 2315-28) and 4-(4-acetoxy-3-chlorophenoxy)benzoic acid as obtained in Preparation 33 as starting material in the presence of o-(7-azabenzotriazol-1-yl)-N,N,N′,N′-tetramethyluronium hexafluorophosphate. The reaction was monitored by TLC or LCMS analysis to afford white foam, 32% yield. Purification was undertaken by flash chromatography on silica gel.

¹H NMR (400 MHz, DMSO-d₆) δ: 0.91 (d, 3H), 1.09 (s, 3H), 1.28-1.37 (m, 1H), 1.53 (s, 3H), 1.53-1.70 (m, 1H), 1.76-1.83 (m, 1H), 1.87-1.98 (m, 1H), 2.17-2.25 (m, 1H), 2.26-2.37 (m, 2H), 2.35 (s, 3H), 2.52-2.71 (m, 1H), 3.30-3.40 (m, 1H), 4.23-4.31 (m, 1H), 5.58-5.77 (m, 1H), 5.64 (d, 1H), 5.74 (dd, 1H), 5.91 (dd, 1H), 6.15 (s, 1H), 6.33 (dd, 1H), 7.17 (dd, 1H), 7.20 (dt, 2H), 7.30 (dd, 1H), 7.38 (d, 1H), 7.43 (d, 1H), 7.94 (dt, 2H) ppm.

LRMS (ESI): m/z 717 [M+H]⁺

### Example 1

### cyanomethyl(6alpha,11beta,17alpha)-17-[(4-benzylbenzoyl)oxy]-6,9-difluoro-11-hydroxy-3-oxoandrosta-1,4-diene-17-carboxylate

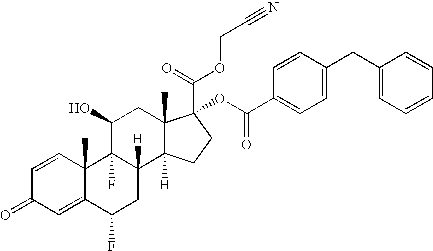

A suspension of (6alpha,11beta,17alpha)-17-[(4-benzylbenzoyl)oxy]-6,9-difluoro-11-hydroxy-3-oxoandrosta-1,4-diene-17-carboxylic acid as obtained in Preparation 2 (366 mg, 0.64 mmol) and sodium hydrogen carbonate (86 mg, 1.00 mmol) in N,N-dimethylformamide (2.5 mL) was cooled to 0° C. and treated with bromoacetonitrile (221 μL, 3.17 mmol). The resulting suspension was allowed to warm to room temperature. After stirring for 18 hours the suspension was treated with bromoacetonitrile (200 μL, 2.87 mmol). After stirring for a further 24 hours the suspension was treated with bromoacetonitrile (200 μL, 2.87 mmol). After stirring for a further 48 hours the suspension was poured into saturated sodium hydrogen carbonate solution (10 mL, aqueous) and extracted with ethyl acetate (3×10 mL). The combined organic fractions were acidified to pH 4 by addition of hydrochloric acid (2N aqueous solution) and washed with water (50 mL) and brine (50 mL) and dried (magnesium sulphate) and concentrated in vacuo. The residue was purified by flash column chromatography on silica gel eluting with heptane:ethyl acetate (1:0 to 1:1, by volume, gradient elution) to give the title compound as a white solid, 52 mg, 13% yield.

¹H NMR (400 MHz, DMSO-d₆) δ: 1.02 (s, 3H), 1.54 (s, 3H), 1.48-1.64 (m, 2H), 1.69-1.80 (m, 2H), 1.93-2.01 (m, 1H), 2.18-2.36 (m, 3H), 2.59-2.74 (m, 1H), 2.86-2.93 (m, 1H), 4.01 (s, 2H), 4.28-4.33 (m, 1H), 5.06 (d, 2H), 5.67-5.69 (m, 1H), 5.60-5.77 (m, 1H), 6.15 (s, 1H), 6.35 (dd, 1H), 7.18-7.33 (m, 6H), 7.42-7.45 (m, 2H), 7.78-7.81 (m, 2H) ppm.

LRMS (ESI): m/z 616 [M+H]⁺

### Example 2

### Cyanomethyl(6alpha,11beta,17alpha)-17-[(biphenyl-4-ylcarbonyl)oxy]-6,9-difluoro-11-hydroxy-3-oxoandrosta-1,4-diene-17-carboxylate

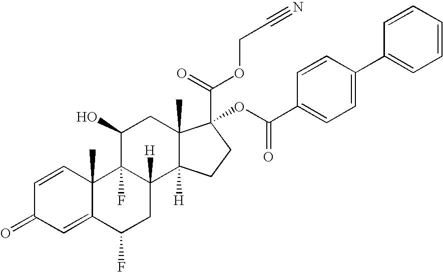

A suspension of (6alpha, 11beta, 17alpha)-6,9-difluoro-11,17-dihydroxy-3-oxoandrosta-1,4-diene-17-carboxylic acid as obtained in Preparation 1 (259 mg, 0.68 mmol) in acetone (5 mL) was cooled over ice and treated with biphenyl-4-carbonyl chloride (151 mg, 0.70 mmol) and pyridine (55 μL, 0.68 mmol). The resulting solution was allowed to warm to ambient temperature over 4 hours with stirring before being acidified to pH 2 by addition of hydrochloric acid (2N aqueous solution) and extracted with ethyl acetate (4×10 mL). The combined organic extracts were washed with hydrochloric acid (2N aqueous solution, 15 mL) and brine (50 mL), dried (magnesium sulphate) and concentrated in vacuo. An ice cooled solution of the residue in N,N-dimethylformamide (22 mL) was treated with sodium hydrogen carbonate (58 mg, 0.69 mmol) and bromoacetonitrile (110 μL, 1.58 mmol). The resulting suspension was allowed to warm to ambient temperature and was stirred for 3 days. The suspension was treated with sodium hydrogen carbonate (55 mg, 0.65 mmol) and bromoacetonitrile (110 μL, 1.58 mmol) and stirred at ambient temperature for 4.5 hours after which time further bromoacetonitrile (110 μL, 1.58 mmol) was added. After stirring at ambient temperature for 18 hours the suspension was treated with hydrochloric acid (2N aqueous solution, 7 mL) and water (8 mL) and extracted with ethyl acetate (3×20 mL). The combined organic layers were washed with saturated sodium hydrogen carbonate solution (50 mL, aqueous), brine (50 mL), dried (magnesium sulphate) and concentrated in vacuo. The residue was purified by flash column chromatography on silica gel eluting with heptane:ethyl acetate (1:0 to 0:1, by volume, gradient elution) to give the title compound as a white solid, 86 mg, 21% yield.

¹H NMR (400 MHz, DMSO-d₆) δ: 1.05 (s, 3H), 1.55 (s, 3H), 1.51-1.66 (m, 2H), 1.73-1.85 (m, 2H), 1.99-2.07 (m, 1H), 2.24-2.37 (m, 3H), 2.62-2.77 (m, 1H), 2.89-2.97 (m, 1H), 4.32-4.37 (m, 1H), 5.10 (d, 2H), 5.70-5.72 (m, 1H), 5.61-5.78 (m, 1H), 6.16 (m, 1H), 6.37 (dd, 1H), 7.32-7.35 (m, 1H), 7.43-7.54 (m, 3H), 7.73-7.76 (m, 2H), 7.88-7.91 (m, 2H), 7.95-7.97 (m, 2H) ppm.

LRMS (ESI): m/z 602 [M+H]⁺

### Examples 3-6

The following compounds of the general formula shown below were prepared by a method similar to that described for Example 2 using the appropriate starting material and (6alpha,11beta,17alpha)-6,9-difluoro-11,17-dihydroxy-3-oxoandrosta-1,4-diene-17-carboxylic acid as obtained in Preparation 1 followed by reaction with bromoacetonitrile. The reactions were monitored by TLC or LCMS analysis. Acid chlorides were commercially available or prepared by a method similar to that described for Preparation 5 (from the corresponding carboxylic acid precursors by treatment with oxalyl chloride in dichloromethane in the presence of a catalytic amount of dimethylformamide followed by concentration in vacuo and used without isolation or purification). Carboxylic acid precursor in Example 6 was as obtained in Preparation 16.

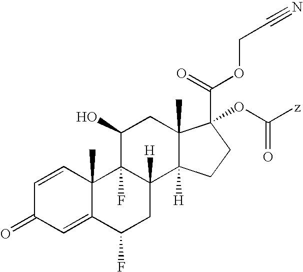

| No | Z | Name and NMR | Yield | LRMS |
|---|---|---|---|---|
| 3 | 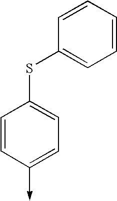 | cyanomethyl (6alpha,11beta,17alpha)-6,9- difluoro-11-hydroxy-3-oxo-17-{[4- (phenylthio)benzoyl]oxy}androsta-1,4-diene- 17-carboxylate Starting material: 4-(phenylthio)benzoic acid ¹H NMR (400 MHz, DMSO-d₆) δ: 1.02 (s, 3H), 1.54 (s, 3H), 1.48-1.64 (m, 2H), 1.68-1.81 (m, 2H), 1.93-2.01 (m, 1H), 2.16-2.36 (m, 3H), 2.59-2.73 (m, 1H), 2.86-2.93 (m, 1H), 4.27-4.33 (m, 1H), 5.07 (d, 2H), 5.67-5.68 (m, 1H), 5.59-5.76 (m, 1H), 6.14 (m, 1H), 6.34 (dd, 1H), 7.29-7.32 (m, 3H), 7.47-7.53 (m, 5H), 7.76-7.80 (m, 2H) ppm. | 67% | (ESI): m/z 634 [M + H]⁺ 678 [M + formate]⁺ |
| 4 | 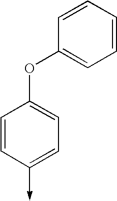 | cyanomethyl (6alpha,11beta,17alpha)-6,9- difluoro-11-hydroxy-3-oxo-17-[(4- phenoxybenzoyl)oxy]androsta-1,4-diene-17- carboxylate Starting material: 4-phenoxybenzoic acid ¹H NMR (400 MHz, DMSO-d₆) δ: 1.03 (s, 3H), 1.54 (s, 3H), 1.49-1.61 (m, 2H), 1.70-1.82 (m, 2H), 1.93-2.02 (m, 1H), 2.18-2.37 (m, 3H), 2.60-2.75 (m, 1H), 2.87-2.95 (m, 1H), 4.27-4.33 (m, 1H), 5.08 (d, 2H), 5.68-5.69 (m, 1H), 5.60-5.78 (m, 1H), 6.14 (m, 1H), 6.34 (dd, 1H), 7.09-7.14 (m, 4H), 7.23-7.33 (m, 2H), 7.44-7.49 (m, 2H), 7.86-7.90 (m, 2H) ppm. | 48% | (ESI): m/z 618 [M + H]⁺ |
| 5 | 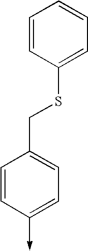 | cyanomethyl (6alpha,11beta,17alpha)-6,9- difluoro-11-hydroxy-3-oxo-17-({4- [(phenylthio)methyl]benzoyl}oxy)androsta- 1,4-diene-17-carboxylate Starting material: 4- [(phenylthio)methyl]benzoic acid₋[DeGraw, Journal of Medicinal Chemistry, 1984, 376-380] ¹H NMR (400 MHz, DMSO-d₆) δ: 1.02 (s, 3H), 1.54 (s, 3H), 1.49-1.65 (m, 2H), 1.69-1.82 (m, 2H), 1.93-2.02 (m, 1H), 2.17-2.36 (m, 3H), 2.57-2.75 (m, 1H), 2.85-2.94 (m, 1H), 4.32 (s, 2H), 4.28-4.34 (m, 1H), 5.07 (d, 2H), 5.68-5.69 (m, 1H), 5.59-5.78 (m, 1H), 6.15 (m, 1H), 6.35 (dd, 1H), 7.16-7.20 (m, 1H), 7.26-7.34 (m, 5H), 7.52-7.54 (m, 2H), 7.77-7.79 (m, 2H) ppm. | 64% | (ESI): m/z 648 [M + H]⁺ |
| 6 | 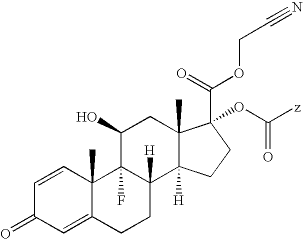 | cyanomethyl (6alpha,11beta,17alpha)-6,9- difluoro-11-hydroxy-17-({4-[(4- hydroxybenzyl)thio]benzoyl}oxy)-3- oxoandrosta-1,4-diene-17-carboxylate Starting material: the compound as obtained in preparation 16 ¹H NMR (400 MHz, DMSO-d₆) δ: 1.03 (s, 3H), 1.54 (s, 3H), 1.48-1.62 (m, 2H), 1.68-1.83 (m. 2H), 1.93-1.99 (m, 1H), 2.17-2.36 (m, 3H), 2.56-2.75 (m, 1H), 2.85-2.94 (m, 1H), 4.24 (s, 2H), 4.29-4.34 (m, 1H), 5.07 (d, 2H), 5.68-5.69 (m, 1H), 5.60-5.78 (m, 1H), 6.15 (m, 1H), 6.36 (dd, 1H), 6.68-6.72 (m, 2H), 7.19-7.23 (m, 2H), 7.31-7.34 (m, 1H), 7.46-7.49 (m, 2H), 7.73-7.76 (m, 2H), 9.39 (s, 1H) ppm. | 39% | (ESI): m/z 664 [M + H]⁺ (API): m/z 662 [M − H]⁻ |

### Examples 7-10

The following compounds of the general formula shown below were prepared by a method similar to that described for Example 2 using the appropriate starting material and (11beta,17alpha)-9-fluoro-11,17-dihydroxy-3-oxoandrosta-1,4-diene-17-carboxylic acid [Phillipps et al. Journal of Medicinal Chemistry, 1994, pages 3717-3729] followed by reaction with bromoacetonitrile. The reactions were monitored by TLC or LCMS analysis.

Acid chlorides were commercially available or prepared by a method similar to that described for Preparation 5 (from the corresponding carboxylic acid precursors by treatment with oxalyl chloride in dichloromethane in the presence of a catalytic amount of dimethylformamide followed by concentration in vacuo and were used without isolation or purification). Carboxylic acid precursor in Example 10 was as obtained in Preparation 41.

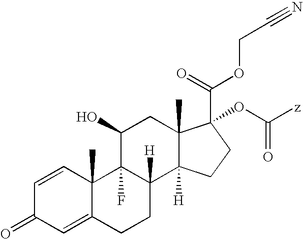

| No | Z | Name and NMR | Yield | LRMS |
|---|---|---|---|---|
| 7 | 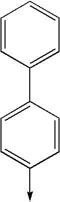 | cyanomethyl (11beta,17alpha)-17-[(biphenyl-4- ylcarbonyl)oxy]-9-fluoro-11-hydroxy-3- oxoandrosta-1,4-diene-17-carboxylate Starting material: Biphenyl-4-carboxylic acid ¹H NMR (400 MHz, CDCl₃) δ: 1.15 (s, 3H), 1.61 (s, 3H), 1.51-1.87 (m, 4H), 1.93-1.99 (m, 1H), 2.03-2.11 (m, 1H), 2.31-2.60 (m, 3H), 2.62-2.73 (m, 2H), 3.04-3.12 (m, 1H), 4.53-4.57 (m, 1H), 4.67 (d, 1H), 4.94 (d, 1H), 6.18 (m, 1H), 6.41 (dd, 1H), 7.24-7.27 (m, 1H), 7.39-7.43 (m, 1H), 7.45-7.50 (m, 2H), 7.59-7.62 (m, 2H), 7.66-7.69 (m, 2H), 8.00-8.03 (m, 2H) ppm. | 33% | (ESI): m/z 584 [M + H]⁺ |
| 8 | 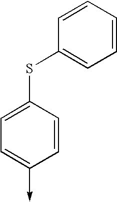 | cyanomethyl (11beta,17alpha)-9-fluoro-11- hydroxy-3-oxo-17-{[4- (phenylthio)benzoyl]oxy}androsta-1,4-diene-17- carboxylate Starting material: 4-(phenylthio)benzoic acid ¹H NMR (400 MHz, CDCl₃) δ: 1.12 (s, 3H), 1.58 (s, 3H), 1.48-1.83 (m, 4H), 1.90-2.03 (m, 2H), 2.21-2.31 (m, 1H), 2.39-2.58 (m, 3H), 2.62-2.71 (m, 1H), 3.00-3.07 (m, 1H), 4.47-4.52 (m, 1H), 4.64 (d, 1H), 4.90 (d, 1H), 6.16 (m, 1H), 6.38 (dd, 1H), 7.14-7.17 (m, 2H), 7.20-7.23 (m, 1H), 7.39-7.43 (m, 3H), 7.48-7.51 (m, 2H), 7.75-7.79 (m, 2H) ppm. | 15% | (ESI): m/z 616 [M + H]⁺ |
| 9 | 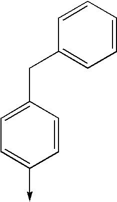 | cyanomethyl (11beta,17alpha)-17-[4- benzylbenzoyl)oxy]-9-fluoro-11-hydroxy-3- oxoandrosta-1,4-diene-17-carboxylate Starting material: 4-benzylbenzoic acid ¹H NMR (400 MHz, CDCl₃) δ: 1.12 (s, 3H), 1.56 (s, 3H), 1.50-1.83 (m, 4H), 1.90-2.06 (m, 2H), 2.26-2.34 (m, 1H), 2.40-2.71 (m, 4H), 3.00-3.08 (m, 1H), 4.02 (s, 2H), 4.49-4.53 (m, 1H), 4.63 (d, 1H), 4.92 (d, 1H), 6.16 (m, 1H), 6.40 (dd, 1H), 7.15-7.31 (m, 8H), 7.84-7.87 (m, 2H) ppm. | 3% | (ESI): m/z 598 [M + H]⁺ |
| 10 | 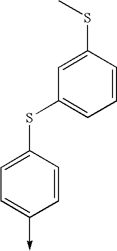 | cyanomethyl (11beta,17alpha)-9-fluoro-11- hydroxy-17-[(4-{[3-(methylthio)- phenyl]thio}benzoyl)oxy]-3-oxoandrosta-1,4- diene-17-carboxylate Starting material: the compound as obtained in preparation 41 ¹H NMR (400 MHz, CDCl₃) δ: 1.12 (s, 3H), 1.59 (s, 3H), 1.48-1.84 (m, 4H), 1.90-2.06 (m, 2H), 2.23-2.31 (m, 1H), 2.48 (s, 3H), 2.40-2.57 (m, 3H), 2.63-2.71 (m, 1H), 3.00-3.07 (m, 1H), 4.48-4.51 (m, 1H), 4.64 (d, 1H), 4.91 (d, 1H), 6.16 (m, 1H), 6.39 (dd, 1H), 7.16-7.19 (m, 2H), 7.21-7.28 (m, 3H), 7.30-7.35 (m, 2H), 7.77-7.80 (m, 2H) ppm. | 30% | (ESI): m/z 662 [M + H]⁺ |

### Example 11

### cyanomethyl(6alpha,11beta,17alpha)-6,9-difluoro-11-hydroxy-17-[(4-{[(4-hydroxyphenyl)thio]methyl}benzoyl)oxy]-3-oxoandrosta-1,4-diene-17-carboxylate

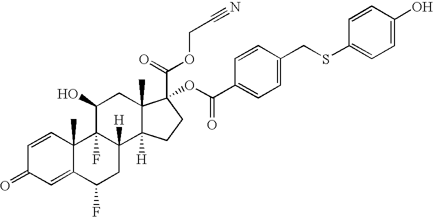

A solution of 4-{[(4-acetoxyphenyl)thio]methyl}benzoic acid as obtained in Preparation 26 (77 mg, 0.26 mmol) in N,N-dimethylformamide (1 mL) was cooled to 0° C. and treated with N-ethyl-N-isopropylpropan-2-amine (108 μL, 0.62 mmol) followed by o-(7-azabenzotriazol-1-yl)-N,N,N′,N′-tetramethyluronium hexafluorophosphate (108 mg, 0.28 mmol). After stirring for 1 hour, the resulting suspension was treated with (6alpha,11beta,17alpha)-6,9-difluoro-11,17-dihydroxy-3-oxoandrosta-1,4-diene-17-carboxylic acid as obtained in Preparation 1 (97 mg, 0.25 mmol) and allowed to warm to ambient temperature. After stirring for 18 hours the resulting suspension was treated with hydrochloric acid (1N aqueous solution, 10 mL). The resulting solid was collected by filtration and washed with water (4×5 mL), suspended in toluene and concentrated in vacuo. The residue was dissolved in N,N-dimethylformamide (0.75 mL), cooled to 0° C. and treated with N-ethyl-N-isopropylpropan-2-amine (31 μL, 0.18 mmol) followed by bromoacetonitrile (13 μL, 0.18 mmol). After stirring for 18 hours the resulting solution was treated with sodium hydrogen carbonate (31 mg, 0.37 mmol), water (0.1 mL) and methanol (1 mL). After stirring for 4 days the suspension was diluted with water (10 mL) and extracted with ethyl acetate (4×5 mL). The combined organic phases were dried (magnesium sulphate) and concentrated in vacuo. The residue was purified by flash column chromatography on silica gel eluting with ethyl acetate:heptane (1:1, by volume) to give the title compound as a white solid, 72 mg, 43% yield.

¹H NMR (400 MHz, DMSO-d₆) δ: 1.03 (s, 3H), 1.54 (s, 3H), 1.48-1.65 (m, 2H), 1.70-1.82 (m, 2H), 1.94-2.02 (m, 1H), 2.18-2.36 (m, 3H), 2.48-2.82 (m, 1H), 2.86-2.93 (m, 1H), 4.11 (s, 2H), 4.30-4.34 (m, 1H), 5.07 (d, 2H), 5.68-5.69 (m, 1H), 5.60-5.78 (m, 1H), 6.15 (s, 1H), 6.34-6.37 (m, 1H), 6.67-6.70 (m, 2H), 7.14-7.18 (m, 2H), 7.31-7.34 (m, 1H), 7.38-7.40 (m, 2H), 7.74-7.77 (m, 2H), 9.56 (s, 1H) ppm.

LRMS (ESI): m/z 664 [M+H]⁺

### Example 12

### (11beta,17alpha)-17-{[(cyanomethyl)thio]-carbonyl}-9-fluoro-11-hydroxy-3-oxoandrosta-1,4-dien-17-yl 4-(3-chloro-4-hydroxyphenoxy)benzoate

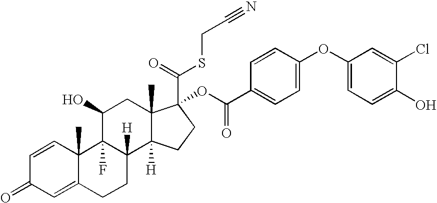

A solution of 4-(4-acetoxy-3-chlorophenoxy)benzoic acid as obtained in Preparation 33 (330 mg, 0.97 mmol) in N,N-dimethylformamide (6 mL) was cooled to 0° C. and treated with o-(7-azabenzotriazol-1-yl)-N,N,N′,N′-tetramethyluronium hexafluorophosphate (388 mg, 1.02 mmol) followed by N-ethyl-N-isopropylpropan-2-amine (372 μL, 2.14 mmol). The suspension was warmed to ambient temperature over 1 hour before being treated with a solution of (11beta,17alpha)-9-fluoro-11,17-dihydroxy-3-oxoandrosta-1,4-diene-17-carbothioic S-acid as obtained in Preparation 3 (370 mg, 0.97 mmol) in N,N-dimethylformamide (6 mL). After the reaction mixture was stirred for 18 hours brine (15 mL) and ethyl acetate (20 mL) were added. The aqueous phase was acidified to pH 4 by the addition of hydrochloric acid (2N aqueous solution) and extracted with ethyl acetate (2×20 mL). The combined organic extracts were dried (sodium sulphate), azeotroped with xylene and concentrated in vacuo. The residue was dissolved in N,N-dimethylformamide (1.0 mL), cooled to 0° C. and treated with N-ethyl-N-isopropylpropan-2-amine (40 μL, 0.23 mmol) followed by bromoacetonitrile (17 μL, 0.26 mmol). After stirring under nitrogen for 24 hours the resulting solution was treated with sodium hydrogen carbonate (90 mg, 1.10 mmol), water (0.33 mL) and methanol (1.4 mL). After stirring for 54 hours the reaction mixture was diluted with water (50 mL), acidified to pH 7 by the addition of hydrochloric acid (2N aqueous solution) and extracted with ethyl acetate (4×50 mL). The combined organics were dried (magnesium sulphate) and concentrated in vacuo co-evaporating with xylene. The residue was purified by flash column chromatography on silica gel eluting with heptane:ethyl acetate (7:3 to 1:1, by volume, gradient elution) to give the title compound as a solid, 16 mg, 5% yield.

¹H NMR (400 MHz, CDCl₃) δ: 1.09 (s, 3H), 1.59 (s, 3H), 1.47-1.66 (m, 2H), 1.75-1.82 (m, 1H), 1.88-1.98 (m, 2H), 2.12-2.28 (m, 2H), 2.39-2.71 (m, 4H), 3.03-3.10 (m, 1H), 3.52 (d, 1H), 3.83 (d, 1H), 4.51-4.55 (m, 1H), 5.82-5.88 (m, 1H), 6.16-6.17 (m, 1H), 6.39 (dd, 1H), 6.90-6.93 (m, 1H), 6.94-6.97 (m, 2H), 7.04-7.07 (m, 2H), 7.24 (d, 1H), 7.89-7.93 (m, 2H) ppm.

LRMS (ESI): m/z 666 [M+H]⁺664 [M−H]⁻

### Example 13

### (11beta,17alpha)-17-{[(cyanomethyl)thio]carbonyl}-9-fluoro-11-hydroxy-3-oxoandrosta-1,4-dien-17-yl 4-[(3-chloro-4-hydroxyphenyl)thio]benzoate

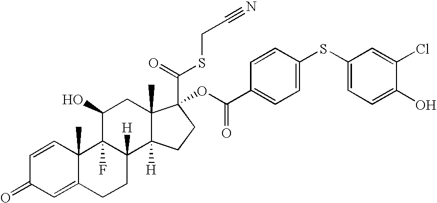

A solution of 4-[(4-acetoxy-3-chlorophenyl)thio]benzoic acid as obtained in Preparation 39 (800 mg, 2.48 mmol) in N,N-dimethylformamide (8 mL) was cooled to 5° C. and stirred under nitrogen before treating with N-ethyl-N-isopropylpropan-2-amine (950 μL, 5.45 mmol) followed by a solution of o-(7-azabenzotriazol-1-yl)-N,N,N′,N′-tetramethyluronium hexafluorophosphate (1.05 g, 2.77 mmol) in N,N-dimethylformamide (2 mL). After stirring at 5° C. for 30 minutes the resulting solution was treated with a solution of (11beta,17alpha)-9-fluoro-11,17-dihydroxy-3-oxoandrosta-1,4-diene-17-carbothioic S-acid as obtained in Preparation 3 (943 mg, 2.48 mmol) in N,N-dimethylformamide (1.5 mL). After stirring for 3 hours at ambient temperature the resulting solution was diluted with hydrochloric acid (2N aqueous solution, 50 mL) and extracted with ethyl acetate (50 mL). The aqueous extract was extracted with ethyl acetate (2×20 mL) and the combined organic extracts were washed with brine (30 mL), dried (magnesium sulphate) and concentrated in vacuo to obtain a yellow oil. This was taken up in ethyl acetate (24 mL) and the resulting white solid precipitate was filtered off. The organic filtrate was concentrated in vacuo to obtain a yellow foam (1.25 g, 1.82 mmol) which was dissolved in N,N-dimethylformamide (6 mL) and N-ethyl-N-isopropylpropan-2-amine (318 μL, 1.82 mmol). The resulting solution was cooled to 5° C. under nitrogen, treated with bromoacetonitrile (127 μL, 1.82 mmol) and stirred at ambient temperature for 20 minutes before the addition of methanol (15 mL) and saturated sodium hydrogen carbonate solution (aqueous, 15 mL). The thick suspension was stirred at ambient temperature for 1.5 hours before being diluted with hydrochloric acid (1N aqueous solution, 100 mL) and ethyl acetate (100 mL). The layers were separated and the aqueous was extracted with ethyl acetate (2×50 mL). The combined organic phases were washed with brine (100 mL), dried (magnesium sulphate) and concentrated in vacuo. The residue was purified by flash column chromatography on silica gel eluting with heptane:ethyl acetate (1:0 to 0:1, by volume, gradient elution) to obtain a yellow foam. This was purified by flash column chromatography on silica gel eluting with toluene:acetone (1:0 to 1:1, by volume, gradient elution) to give an off white foam. Further separation was undertaken (Chiralpak IA column (250×20 mm i.d.), Flow 18 mL/min, Ambient temperature, Eluent: MeOH/EtOH 1:1, Sample dissolution: 200 mg in 5 mL MeOH/EtOH (1:1), Maximum injection volume: 500 μL) to give the title compound as an off white solid, 81 mg, 10% yield.

¹H NMR (400 MHz, DMSO-d₆) δ: 0.98 (s, 3H), 1.36-1.53 (m, 2H), 1.53 (s, 3H), 1.67-1.76 (m, 1H), 1.84-1.98 (m, 2H), 2.02-2.13 (m, 2H), 2.30-2.38 (m, 2H), 2.44-2.60 (m, 1H), 2.62-2.71 (m, 1H), 2.85-2.93 (m, 1H), 3.96 (d, 1H), 4.05 (d, 1H), 4.31-4.34 (m, 1H), 5.60-5.61 (m, 1H), 6.05 (m, 1H), 6.27 (dd, 1H), 7.08 (d, 1H), 7.19-7.28 (m, 2H), 7.31-7.36 (m, 2H), 7.54 (d, 1H), 7.76-7.79 (m, 2H) 10.80 (br s, 1H) ppm.

LRMS (ESI): m/z 682 [M+H]⁺

### Example 14

### cyanomethyl(11beta,17alpha)-9-fluoro-11-hydroxy-3-oxo-17-[(4-phenoxybenzoyl)oxy]androsta-1,4-diene-17-carboxylate

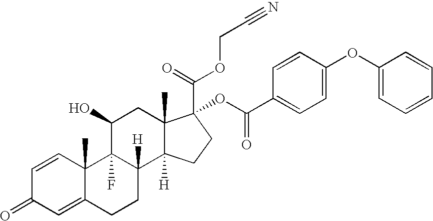

The title compound was prepared by a method similar to that described for Example 13 using 4-phenoxybenzoic acid as starting material and (11beta,17alpha)-9-fluoro-11,17-dihydroxy-3-oxoandrosta-1,4-diene-17-carboxylic acid [Phillipps et al, Journal of Medicinal Chemistry, 1994, pages 3717-3729] followed by reaction with bromoacetonitrile. The reactions were monitored by TLC or LCMS analysis. The title compound was obtained with a yield of 67%.

¹H NMR (400 MHz, DMSO-d₆) δ: 1.04 (s, 3H), 1.54 (s, 3H), 1.39-1.58 (m, 2H), 1.69-1.79 (m, 2H), 1.86-2.01 (m, 2H), 2.10-2.18 (m, 1H), 2.27-2.40 (m, 2H), 2.47-2.73 (m, 2H), 2.85-2.93 (m, 1H), 4.27-4.33 (m, 1H), 5.07 (m, 2H), 5.58-5.59 (m, 1H), 6.05 (m, 1H), 6.27 (dd, 1H), 7.09-7.14 (m, 4H), 7.23-7.28 (m, 1H), 7.32-7.35 (m, 1H), 7.44-7.49 (m, 2H), 7.86-7.90 (m, 2H) ppm.

(ESI): m/z 600 [M+H]⁺

### Example 15

### cyanomethyl(11beta,17alpha)-9-fluoro-11-hydroxy-17-({4-[(3-hydroxyphenyl)thio]-benzoyl}oxy)-3-oxoandrosta-1,4-diene-17-carboxylate

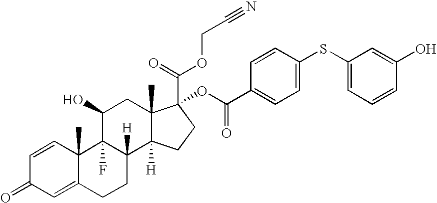

A solution of cyanomethyl(11beta,17alpha)-17-({4-[(3-acetoxyphenyl)thio]benzoyl}oxy)-9-fluoro-11-hydroxy-3-oxoandrosta-1,4-diene-17-carboxylate as obtained in Preparation 54 (129 mg, 0.19 mmol) in methanol (10 mL) and water (0.5 mL) was treated with sodium hydrogen carbonate (70 mg, 0.83 mmol) and stirred at ambient temperature for 20 hours. The resulting suspension was diluted with brine (5 mL) and extracted with ethyl acetate (30 mL). The aqueous layer was acidified to pH 4 by the addition of hydrochloric acid (2N aqueous solution) and extracted with ethyl acetate (2×20 mL). The combined organic extracts were dried (magnesium sulphate) and concentrated in vacuo. The residue was purified by flash column chromatography on silica gel eluting with dichloromethane:ethyl acetate:acetic acid (80:20:0 to 280:120:1 by volume, gradient elution) to give the title compound as a white solid, 27 mg, 22% yield.

¹H NMR (400 MHz, CDCl₃) δ: 1.12 (s, 3H), 1.48-1.82 (m, 3H), 1.57 (s, 3H), 1.91-2.07 (m, 2H), 2.21-2.29 (m, 1H), 2.36-2.56 (m, 4H), 2.62-2.70 (m, 1H), 2.99-3.06 (m, 1H), 4.43-4.45 (m, 1H), 4.66 (d, 1H), 4.89 (d, 1H), 6.18 (s, 1H), 6.35-6.38 (m, 1H), 6.89-6.92 (m, 1H), 7.00-7.03 (m, 2H), 7.16-7.25 (m, 4H), 7.76-7.79 (m, 2H) ppm.

LRMS (ESI): m/z 632 [M+H]⁺

### Examples 16-20

The following compounds of the general formula shown below were prepared by a method similar to that described for Example 15 by treatment of the appropriate starting material with sodium hydrogen carbonate and water in methanol solution (tetrahydrofuran added as a co-solvent in Examples 18-20). The reactions were monitored by TLC or LCMS analysis.

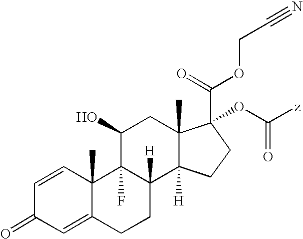

| No | Z | Name and NMR | Yield | LRMS |
|---|---|---|---|---|
| 16 | 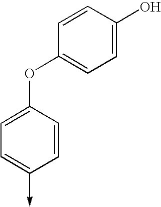 | cyanomethyl (11beta,17alpha)-9-fluoro-11- hydroxy-17-{[4-(4- hydroxyphenoxy)benzoyl]oxy}-3- oxoandrosta-1,4-diene-17-carboxylate Starting material: the compound as obtained in preparation 48. ¹H NMR (400 MHz, CDCl₃) δ: 1.19 (s, 3H), 1.48-1.67 (m, 2H), 1.60 (s, 3H), 1.72-1.80 (m, 1H), 1.91-2.10 (m, 2H), 2.25-2.33 (m, 1H), 2.39-2.58 (m, 4H), 2.63-2.73 (m, 1H), 2.95-3.07 (m, 1H), 4.38 (d, 1H), 4.45-4.47 (m, 1H), 4.95 (d, 1H), 6.18 (m, 1H), 6.38-6.41 (m, 1H), 6.86-6.92 (m, 6H), 7.27-7.30 (m, 1H), 7.86-7.89 (m, 2H) ppm. | 29% | (ESI): m/z 616 [M + H]⁺ |
| 17 | 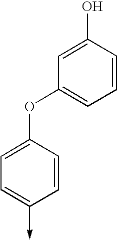 | cyanomethyl (11beta,17alpha)-9-fluoro-11- hydroxy-17-{[4-(3- hydroxyphenoxy)benzoyl]oxy}-3- oxoandrosta-1,4-diene-17-carboxylate Starting material: the compound as obtained in preparation 53. ¹H NMR (400 MHz, CDCl₃) δ: 1.13 (s, 3H), 1.58 (s, 3H), 1.49-1.84 (m, 4H), 1.91-2.07 (m, 2H), 2.25-2.33 (m, 1H), 2.40-2.58 (m, 3H), 2.63-2.71 (m, 1H), 3.00-3.08 (m, 1H), 4.46-4.49 (m, 1H), 4.66 (d, 1H), 4.92 (d, 1H), 6.12 (s, 1H), 6.17 (m, 1H), 6.36-6.39 (m, 1H), 6.56-6.60 (m, 2H), 6.68-6.71 (m, 1H), 6.98-7.01 (m, 2H), 7.20-7.24 (m, 2H), 7.88-7.91 (m, 2H) ppm. | 24% | (ESI): m/z 616 [M + H]⁺ |
| 18 | 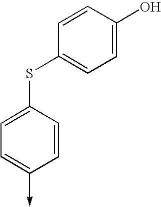 | cyanomethyl (11beta,17alpha)-9-fluoro-11- hydroxy-17-({4-[(4- hydroxyphenyl)thio]benzoyl}oxy)-3- oxoandrosta-1,4-d iene-17-carboxylate Starting material: the compound as obtained in preparation 49. ¹H NMR (400 MHz, CDCl₃) δ: 1.12 (s, 3H), 1.47-1.67 (m, 1H), 1.59 (s, 3H), 1.69-1.73 (m, 1H), 1.74-1.82 (m, 1H), 1.90-2.05 (m, 3H), 2.22-2.30 (m, 1H), 2.41-2.56 (m, 3H), 2.63-2.71 (m, 1H), 2.99-3.07 (m, 1H), 4.46-4.50 (m, 1H), 4.63 (d, 1H), 4.91 (d, 1H), 6.19 (m, 1H), 6.40-6.43 (m, 1H), 6.90-6.94 (m, 2H), 7.03-7.07 (m, 2H), 7.26-7.28 (m, 1H), 7.38-7.42 (m, 2H), 7.73-7.76 (m, 2H) ppm. | 75% | (ESI): m/z 632 [M + H]⁺ |
| 19 | 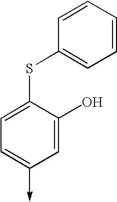 | cyanomethyl (11beta,17alpha)-9-fluoro-11- hydroxy-17-{[3-hydroxy-4- (phenylthio)benzoyl]oxy}-3-oxoandrosta- 1,4-diene-17-carboxylate Starting material: the compound as obtained in preparation 55. ¹H NMR (400 MHz, CDCl₃) δ: 1.13 (s, 3H), 1.59 (s, 3H), 1.49-1.85 (m, 4H), 1.91-2.07 (m, 2H), 2.24-2.34 (m, 1H), 2.40-2.57 (m, 3H), 2.63-2.71 (m, 1H), 3.01-3.12 (m, 1H), 4.49-4.51 (m, 1H), 4.66 (d, 1H), 4.90 (d, 1H), 6.16 (m, 1H), 6.36-6.39 (m, 1H), 6.98 (br s, 1H), 7.20-7.31 (m, 6H), 7.38-7.46 (m, 2H), 7.55-7.56 (m, 1H) ppm. | 47% | (ESI): m/z 632 [M + H]⁺ |
| 20 | 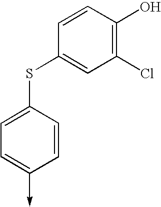 | cyanomethyl (11beta,17alpha)-17-({4-[(3- chloro-4-hydroxyphenyl)thio]-benzoyl}oxy)- 9-fluoro-11-hydroxy-3-oxoandrosta-1,4- diene-17-carboxylate Starting material: the compound as obtained in preparation 50. ¹H NMR (400 MHz, CDCl₃) δ: 1.12 (s, 3H), 1.59 (s, 3H), 1.47-1.83 (m, 3H), 1.90-2.05 (m, 3H), 2.22-2.30 (m, 1H), 2.41-2.57 (m, 3H), 2.63-2.72 (m, 1H), 3.00-3.07 (m, 1H), 4.48-4.51 (m, 1H), 4.64 (d, 1H), 4.90 (d, 1H), 6.18 (s, 1H), 6.34 (br s, 1H), 6.39-6.42 (m, 1H), 7.07-7.10 (m, 3H), 7.24-7.27 (m, 1H), 7.33-7.35 (m, 1H), 7.51-7.52 (m, 1H), 7.76-7.79 (m, 2H) ppm. | 57% | (ESI): m/z 666 [M + H]⁺ |

### Example 21

### cyanomethyl(11beta,17alpha)-9-fluoro-11-hydroxy-17-[(2-hydroxy-4-phenoxybenzoyl)oxy]-3-oxoandrosta-1,4-diene-17-carboxylate

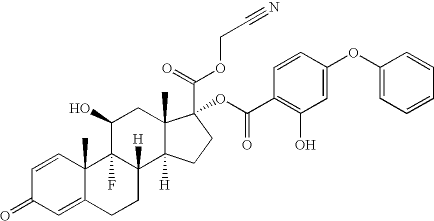

A solution of 2-acetoxy-4-phenoxybenzoic acid as obtained in Preparation 28 (350 mg, 1.29 mmol) in dichloromethane (5 mL) was treated with oxalyl chloride (233 μL, 2.76 mmol) and dimethylformamide (50 μL). After stirring at ambient temperature for 1 hour the solution was concentrated in vacuo. The residue was dissolved in acetone (3 mL) and added dropwise to a cooled suspension (0° C.) of (11beta,17alpha)-9-fluoro-11,17-dihydroxy-3-oxoandrosta-1,4-diene-17-carboxylic acid [Phillipps et al, Journal of Medicinal Chemistry, 1994, pages 3717-3729] (335 mg, 0.92 mmol) in acetone (3 mL). The resulting suspension was treated with the dropwise addition of pyridine (89 μL, 1.10 mmol) and stirred for 20 hours before the dropwise addition of diethylamine (475 μL, 4.60 mmol). The solution was stirred at ambient temperature for 17 hours before being diluted with ethyl acetate (50 mL) and water (50 mL). The organic extract was washed with water (50 mL), brine (2×50 mL), dried (sodium sulphate) and concentrated in vacuo. The residue was dissolved in dimethylformamide (4 mL) and treated with sodium hydrogen carbonate (82 mg, 978 μmol) and the dropwise addition of bromoacetonitrile (68 μL, 978 μmol). After stirring at ambient temperature for 20 hours the solution was diluted with ethyl acetate (50 mL) and saturated sodium hydrogen carbonate solution (50 mL, aqueous). The organic extract was washed with water (50 mL), brine (50 mL), dried (sodium sulphate) and concentrated in vacuo. The residue was dissolved in methanol (2 mL) and water (250 μL) and treated with sodium hydrogen carbonate (252 mg, 3.00 mmol). After stirring at ambient temperature for 20 hours the solution was diluted with ethyl acetate (50 mL) and water (50 mL). Organic extract washed with water (50 mL), brine (50 mL), dried (sodium sulphate) and concentrated in vacuo. The residue was recrystallised from methanol (10 ml/g) to afford the title compound as a colourless crystalline solid, 170 mg, 30% yield.

¹H NMR (400 MHz, CDCl₃) δ: 1.13 (s, 3H), 1.59 (s, 3H), 1.50-1.74 (m, 3H), 1.78-1.86 (m, 1H), 1.91-1.97 (m, 1H), 2.01-2.09 (m, 1H), 2.23-2.31 (m, 1H), 2.39-2.58 (m, 3H), 2.63-2.71 (m, 1H), 3.01-3.09 (m, 1H), 4.50-4.52 (m, 1H), 4.67 (d, 1H), 4.92 (d, 1H), 6.16 (m, 1H), 6.37-6.40 (m, 1H), 6.45-6.51 (m, 2H), 7.04-7.07 (m, 2H), 7.20-7.26 (m, 2H), 7.37-7.42 (m, 2H), 7.64 (d, 1H), 10.40 (s, 1H) ppm.

LRMS (ESI) 616 [M+H]⁺

### Example 22

### (11beta,17alpha)-9-fluoro-17-{[(fluoromethyl)thio]carbonyl}-11-hydroxy-3-oxoandrosta-1,4-dien-17-yl 4-benzylbenzoate

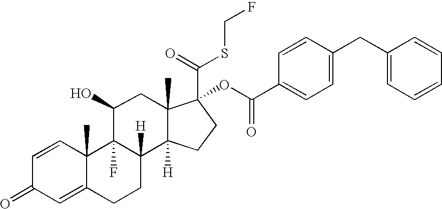

A solution of (11beta,17alpha)-17-[(4-benzylbenzoyl)oxy]-9-fluoro-11-hydroxy-3-oxoandrosta-1,4-diene-17-carbothioic S-acid as obtained in Preparation 4 (129 mg, 0.22 mmol) in acetonitrile (4 mL) was treated with N-ethyl-N-isopropylpropan-2-amine (98 μL, 0.56 mmol) and water (0.3 mL) and cooled to 0° C. Bromofluoromethane was bubbled through the resulting suspension for 7 minutes. The resulting suspension was transferred to a sealed tube and heated to 50° C. for 18 hours before being diluted with hydrochloric acid (0.5N aqueous solution, 15 mL) and extracted with ethyl acetate (2×15 mL). The combined organic extracts were washed with brine (15 mL), dried (sodium sulphate) and concentrated in vacuo to yield the title compound as a white solid, 122 mg, 90% yield.

¹H NMR (400 MHz, CDCl₃) δ: 1.07 (s, 3H), 1.44-1.80 (m, 3H), 1.58 (s, 3H), 1.89-2.00 (m, 2H), 2.08-2.18 (m, 1H), 2.22-2.31 (m, 1H), 2.39-2.56 (m, 2H), 2.62-2.70 (m, 2H), 3.04-3.12 (m, 1H), 4.03 (s, 2H), 4.51-4.55 (m, 1H), 5.64 (dd, 1H), 6.01 (dd, 1H), 6.16 (m, 1H), 6.39 (dd, 1H), 7.15-7.31 (m, 8H), 7.87-7.90 (m, 2H) ppm.

LRMS (ESI): m/z 607 [M+H]⁺

### Example 23

### (11beta,17alpha)-9-fluoro-17-{[(fluoromethyl)thio]carbonyl}-11-hydroxy-3-oxoandrosta-1,4-dien-17-yl 4-{[3-(methylthio)phenyl]thio}benzoate

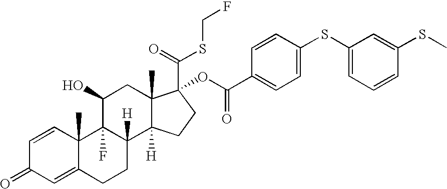

A solution of 4-{[3-(methylthio)phenyl]thio}benzoic acid as obtained in Preparation 41 (145 mg, 0.53 mmol) in dimethylformamide (3 mL) was treated with N-ethyl-N-isopropylpropan-2-amine (224 μL, 1.28 mmol) followed by o-(7-azabenzotriazol-1-yl)-N,N,N′,N′-tetramethyluronium hexafluorophosphate (224 mg, 0.59 mmol) and stirred at ambient temperature for 10 minutes. The resulting solution was treated with (11beta,17alpha)-9-fluoro-11,17-dihydroxy-3-oxoandrosta-1,4-diene-17-carbothioic S-acid as obtained in Preparation 3 (200 mg, 0.53 mmol) and stirred for 18 hours at ambient temperature. Hydrochloric acid (2N aqueous solution, 10 mL) and water (10 mL) were added and the resulting solid was collected by filtration and washed with water (10 mL). The crude solid was dissolved in methanol (20 mL), concentrated in vacuo and purified by flash column chromatography on silica gel eluting with heptane:ethyl acetate:methanol:acetic acid (400:80:20:1 to 80:20:1 by volume, gradient elution) giving the intermediate as a white solid. This solid (120 mg) was dissolved in acetonitrile (3 mL) and water (250 μL) and cooled to 0° C. before being treated with N-ethyl-N-isopropylpropan-2-amine (98 μL, 0.56 mmol). Bromo(fluoro)methane was bubbled through the solution for 4 minutes before the reaction mixture was transferred to a sealed tube and heated to 75° C. for 1 hour. The reaction mixture was allowed to cool to ambient temperature, diluted with hydrochloric acid (0.5M aqueous solution, 15 mL) and extracted with ethyl acetate (2×15 mL). The combined organic extracts were washed with brine (30 mL), dried (sodium sulphate) and concentrated in vacuo. The residue was purified by flash column chromatography on silica gel eluting with heptane:ethyl acetate (9:1 changing to 0:1, by volume, gradient elution) and then recrystallised from a mixture of heptane and ethyl acetate (4:1 ratio) to give the title compound as a white solid, 61 mg, 17% yield, (4:3 solvate with ethyl acetate).

¹H NMR (400 MHz, DMSO-d₆) δ: 0.97 (s, 3H), 1.16 (t, ethyl acetate), 1.36-1.53 (m, 2H), 1.53 (s, 3H), 1.68-1.76 (m, 1H), 2.01 (s, ethyl acetate), 1.84-1.95 (m, 2H), 2.04-2.12 (m, 2H), 2.32-2.38 (m, 2H), 2.45-2.71 (m, 2H), 2.49 (s, 3H), 2.86-2.93 (m, 1H), 4.05 (q, ethyl acetate), 4.30-4.34 (m, 1H), 5.57-5.59 (m, 1H), 5.81-5.88 (m, 1H), 5.93-6.00 (m, 1H), 6.05 (s, 1H), 6.27 (dd, 1H), 7.24-7.27 (m, 1H), 7.31-7.37 (m, 5H), 7.40-7.44 (m, 1H), 7.80-7.84 (m, 2H) ppm.

LRMS (ESI): m/z 671 [M+H]⁺

### Example 24

### (11beta,17alpha)-9-fluoro-17-{[(fluoromethyl)thio]carbonyl}-11-hydroxy-3-oxoandrosta-1,4-dien-17-yl 4-(phenylthio)benzoate

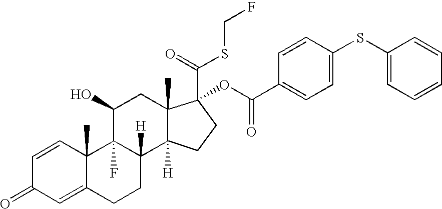

A suspension of (11beta,17alpha)-9-fluoro-11-hydroxy-3-oxo-17-{[4-(phenylthio)-benzoyl]oxy}androsta-1,4-diene-17-carbothioic S-acid as obtained in Preparation 56 (150 mg, 0.25 mmol) in 2-butanone (1 mL) was cooled to 0° C. and treated with a solution of bromo(fluoro)methane in 2-butanone (1M, 1 mL, 1 mmole) followed by sodium iodide (150 mg, 0.80 mmol) and N-ethyl-N-isopropylpropan-2-amine (50 μL, 0.38 mmol). The resulting suspension was stirred at 0° C. for 5 minutes then allowed to warm to ambient temperature. After stirring for 2 hours at ambient temperature the resulting suspension was diluted with ethyl acetate (5 mL) and washed with sodium bisulfite (10% w/v aqueous solution, 5 mL), hydrochloric acid (2M solution, 5 mL), water (5 mL) and brine (5 mL). The organic phase was dried (magnesium sulphate) and concentrated in vacuo. The residue was recrystallised from a mixture of ethyl acetate and heptane (1:1) to give the title compound as a white solid, 80 mg, 51% yield.

¹H NMR (400 MHz, DMSO-d₆) δ: 0.97 (s, 3H), 1.36-1.54 (m, 2H), 1.54 (s, 3H), 1.68-1.76 (m, 1H), 1.84-1.96 (m, 2H), 2.04-2.12 (m, 2H), 2.31-2.39 (m, 2H), 2.44-2.72 (m, 2H), 2.86-2.94 (m, 1H), 4.29-4.35 (m, 1H), 5.55-5.57 (m, 1H), 5.84 (dd, 1H), 5.97 (dd, 1H), 6.05 (m, 1H), 6.27 (dd, 1H), 7.30-7.34 (m, 3H), 7.47-7.54 (m, 5H), 7.79-7.82 (m, 2H) ppm.

LRMS (ESI): m/z 625 [M+H]⁺

### Examples 25-26

The following compounds of the general formula shown below were prepared by a method similar to that described for Example 24 using the appropriate starting material and bromo(fluoro)methane in the presence of sodium iodide. The reactions were monitored by TLC or LCMS analysis. No purification was undertaken on the title compounds.

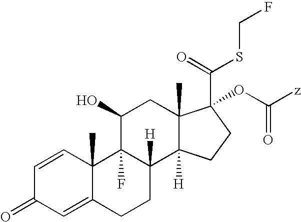

| No | Z | Name and NMR | Yield | LRMS |
|---|---|---|---|---|
| 25 | 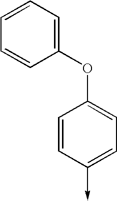 | (11beta, 17alpha)-9-fluoro-17-{[(fluoromethyl)thio]carbonyl}- 11-hydroxy-3-oxoandrosta-1,4-dien-17-yl 4-phenoxybenzoate Starting material: the compound as obtained in preparation 58. ¹H NMR (400 MHz, DMSO-d₆) δ: 0.98 (s, 3H), 1.54 (s, 3H), 1.37- 1.56 (m, 2H), 1.69-1.78 (m, 1H), 1.85-1.97 (m, 2H), 2.05-2.15 (m, 2H), 2.33-2.39 (m, 2H), 2.45-2.72 (m, 2H), 2.87-2.95 (m, 1H), 4.30-4.35 (m, 1H), 5.56-5.57 (m, 1H), 5.82-5.89 (m, 1H), 5.94-6.02 (m, 1H), 6.05 (m, 1H), 6.27 (dd, 1H), 7.10-7.15 (m, 4H), 7.24-7.28 (m, 1H), 7.33 (d, 1H), 7.44-7.50 (m, 2H), 7.89-7.93 (m, 2H) ppm. | 98% | (ESI): m/z 609 [M + H]⁺ |
| 26 | 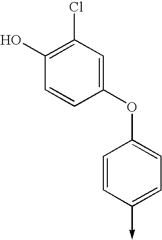 | (11beta, 17alpha)-9-fluoro-17-{[(fluoromethyl)thio]carbonyl}- 11-hydroxy-3-oxoandrosta-1,4-dien-17-y1 4-(3-chloro-4- hydroxyphenoxy)-benzoate Starting material: the compound as obtained in preparation 57. ¹H NMR (400 MHz, CDCl₃) δ: 1.07 (s, 3H), 1.58 (s, 3H), 1.46-1.68 (m, 2H), 1.73-1.83 (m, 1H), 1.89-2.03 (m, 2H), 2.10-2.30 (m, 2H), 2.35-2.57 (m, 2H), 2.58-2.72 (m, 2H), 3.06-3.13 (m, 1H), 4.48-4.57 (m, 1H), 5.58-7.78 (m, 1H), 5.71 (br s, 1H), 5.94-6.09 (m, 1H), 6.16 (m, 1H), 6.39 (dd, 1H), 6.90-6.93(m, 1H), 6.94-6.97 (m, 2H), 7.04-7.08 (m, 2H), 7.23 (d, 1H), 7.91-7.94 (m, 2H) ppm. | 15% | (ESI): m/z 657 [M - H]⁻ |

### Example 27

### (11beta,17alpha)-9-fluoro-17-[((fluoromethyl)thio]carbonyl)-11-hydroxy-3-oxoandrosta-1,4-dien-17-yl 4-[(3-chloro-4-hydroxyphenyl)thio]benzoate

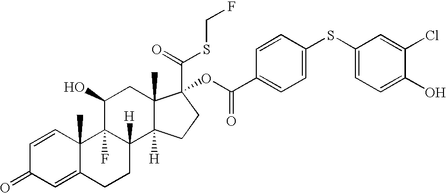

A solution of 4-[(4-acetoxy-3-chlorophenyl)thio]benzoic acid as obtained in Preparation 39 (800 mg, 2.48 mmol) in N,N-dimethylformamide (8 mL) was cooled to 5° C. and stirred under nitrogen before treating with N-ethyl-N-isopropylpropan-2-amine (950 μL, 5.45 mmol) followed by a solution of o-(7-azabenzotriazol-1-yl)-N,N,N′,N′-tetramethyluronium hexafluorophosphate (1.05 g, 2.77 mmol) in N,N-dimethylformamide (2 mL). After stirring at 5° C. for 30 minutes a solution of (11beta,17alpha)-9-fluoro-11,17-dihydroxy-3-oxoandrosta-1,4-diene-17-carbothioic S-acid as obtained in Preparation 3 (943 mg, 2.48 mmol) in N,N-dimethylformamide (1.5 mL) was added. After stirring for 3 hours at ambient temperature the resulting solution was diluted with hydrochloric acid (2N aqueous solution, 50 mL) and extracted with ethyl acetate (50 mL). The aqueous was extracted with ethyl acetate (2×20 mL) and the combined organic extracts were washed with brine (30 mL), dried (magnesium sulphate) and concentrated in vacuo to obtain a yellow oil. This was triturated with ethyl acetate (24 mL) and the resulting white solid precipitate was filtered off. The organic filtrate was concentrated in vacuo to obtain a yellow foam (1.25 g, 1.82 mmol) which was dissolved in N,N-dimethylformamide (750 μL) and N-ethyl-N-isopropylpropan-2-amine (318 μL, 1.82 mmol). The resulting solution was cooled to 5° C. under nitrogen and treated with a solution of bromofluoromethane in 2-butanone (1.42 M, 1.28 mL, 1.82 mmol). The resulting solution was stirred at ambient temperature for 30 minutes before the addition of methanol (15 mL) and saturated sodium hydrogen carbonate solution (15 mL, aqueous). The reaction was stirred at ambient temperature for 1 hour before being diluted with hydrochloric acid (1N aqueous solution, 100 mL) and ethyl acetate (100 mL). The aqueous layer was extracted with ethyl acetate (2×50 mL). The combined organic phases were washed with brine (100 mL), dried (magnesium sulphate) and concentrated in vacuo. The residue was purified by flash column chromatography on silica gel eluting with heptane:ethyl acetate (1:0 to 0:1, by volume, gradient elution) to obtain a white foam. This was purified by flash column chromatography on silica gel eluting with toluene:acetone (1:0 to 7:3, by volume, gradient elution) to give the title compound as a white foam, 70 mg, 8% yield.

¹H NMR (400 MHz, DMSO-d₆) δ: 0.97 (s, 3H), 1.36-1.51 (m, 2H), 1.53 (s, 3H), 1.67-1.76 (m, 1H), 1.83-1.94 (m, 2H), 2.03-2.14 (m, 2H), 2.31-2.38 (m, 2H), 2.44-2.59 (m, 1H), 2.62-2.71 (m, 1H), 2.86-2.92 (m, 1H), 4.29-4.34 (m, 1H), 5.55-5.56 (m, 1H), 5.80-5.87 (m, 1H), 5.92-6.00 (m, 1H), 6.05 (m, 1H), 6.27 (dd, 1H), 7.09 (d, 1H), 7.20-7.23 (m, 2H), 7.31-7.37 (m, 2H), 7.55 (d, 1H), 7.77-7.80 (m, 1H), 10.82 (br s, 1H) ppm.

LRMS (API): m/z 675 [M+H]⁺

### Example 28

### (11beta,17alpha)-17-{[(cyanomethyl)thio]carbonyl}-9-fluoro-11-hydroxy-3-oxoandrosta-1,4-dien-17-yl 4-(phenylthio)benzoate

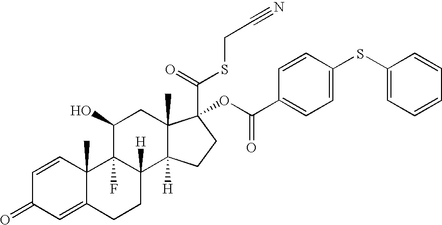

A solution of (11beta,17alpha)-9-fluoro-11-hydroxy-3-oxo-17-{[4-(phenylthio)benzoyl]-oxy}androsta-1,4-diene-17-carbothioic S-acid as obtained in Preparation 56 (78 mg, 130 μmol) in N,N-dimethylformamide (1 mL) was cooled to 0° C. and treated with sodium hydrogen carbonate (59 mg, 700 μmol) followed by bromoacetonitrile (46 μL, 660 μmol). The resulting reaction mixture was warmed to ambient temperature and stirred for 16 hours before diluting with water (10 mL) and extracting with ethyl acetate (4×10 mL). The combined organic phases were dried (magnesium sulphate) and concentrated in vacuo. The residue was purified by flash column chromatography on silica gel eluting with heptane:ethyl acetate (1:1, by volume) to give the title compound as a white solid, 13 mg, 49% yield.

¹H NMR (400 MHz, DMSO-d₆) δ: 0.99 (s, 3H), 1.36-1.53 (m, 2H), 1.53 (s, 3H), 1.68-1.77 (m, 1H), 1.84-1.94 (m, 2H), 2.03-2.15 (m, 2H), 2.30-2.38 (m, 2H), 2.45-2.60 (m, 1H), 2.62-2.71 (m, 1H), 2.86-2.94 (m, 1H), 3.95-4.08 (m, 2H), 4.30-4.36 (m, 1H), 5.60-5.61 (m, 1H), 6.05 (s, 1H), 6.27 (dd, 1H), 7.30-7.34 (m, 3H), 7.48-7.55 (m, 5H), 7.79-7.82 (m, 2H) ppm.

LRMS (ESI): m/z 632 [M+H]⁺

### Example 29

### (11beta,17alpha)-17-{[(cyanomethyl)-thio]carbonyl}-9-fluoro-11-hydroxy-3-oxoandrosta-1,4-dien-17-yl 4-phenoxybenzoate

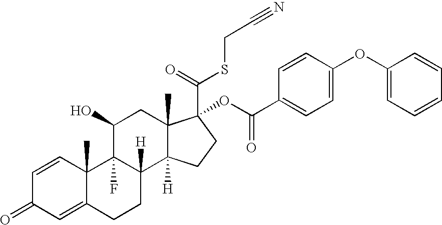

The title compound was prepared by a method similar to that described for Example 28 using the compound as obtained in preparation 58 as starting material in the presence of sodium hydrogen carbonate and bromoacetonitrile. The reaction was monitored by TLC or LCMS analysis. The title compound was obtained with a yield of 72% and no purification was undertaken.

¹H NMR (400 MHz, DMSO-d₆) δ: 1.00 (s, 3H), 1.54 (s, 3H), 1.37-1.56 (m, 2H), 1.70-1.77 (m, 1H), 1.86-1.96 (m, 2H), 2.05-2.15 (m, 2H), 2.33-2.39 (m, 2H), 2.46-2.61 (m, 1H), 2.63-2.75 (m, 1H), 2.88-2.95 (m, 1H), 3.96-4.09 (m, 2H), 4.30-4.36 (m, 1H), 5.61-5.62 (m, 1H), 6.05 (m, 1H), 6.27 (dd, 1H), 7.09-7.15 (m, 4H), 7.24-7.28 (m, 1H), 7.33 (d, 1H), 7.44-7.50 (m, 2H), 7.89-7.93 (m, 2H) ppm.

(ESI): m/z 616 [M+H]⁺

### Example 30

### (11beta,17alpha)-17-{[(chloromethyl)thio]carbonyl}-9-fluoro-11-hydroxy-3-oxoandrosta-1,4-dien-17-yl 4-(phenylthio)benzoate

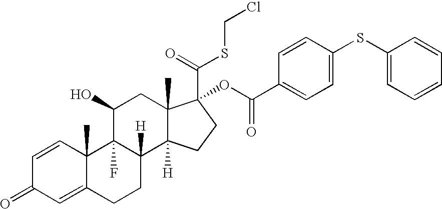

4-(Phenylthio)benzoyl chloride was prepared from 4-(phenylthio)benzoic acid following Preparation 5 by treatment with oxalyl chloride in dichloromethane in the presence of a catalytic amount of N,N-dimethylformamide followed by concentration in vacuo and used without isolation or purification.

A suspension of (11beta,17alpha)-9-fluoro-11,17-dihydroxy-3-oxoandrosta-1,4-diene-17-carbothioic S-acid as obtained in Preparation 3 (612 mg, 1.61 mmol) and 4-(Phenylthio)benzoyl chloride (600 mg, 2.40 mmol) in dichloromethane (25 mL) was treated with triethylamine (674 μL, 4.82 mmol). The solution was stirred at ambient temperature for 2 hours before being diluted with dichloromethane (25 mL) and saturated sodium hydrogen carbonate solution (20 mL, aqueous solution). The organic phase was washed with brine (20 mL) and dried (sodium sulphate) over 4 days before concentrating in vacuo. The residue was purified by flash column chromatography on silica gel eluting with hexane:(methanol:ethyl acetate:acetic acid 20:80:1) (2:8 to 7:3, by volume, gradient elution) to give the title compound as a off-white solid, 844 mg, 82% yield.

¹H NMR (400 MHz, DMSO-d₆) δ: 0.98 (s, 3H), 1.35-1.55 (m, 2H), 1.53 (s, 3H), 1.68-1.76 (m, 1H), 1.82-2.14 (m, 4H), 2.30-2.38 (m, 2H), 2.44-2.59 (m, 1H), 2.62-2.70 (m, 1H), 2.87-2.94 (m, 1H), 4.31-4.34 (m, 1H), 5.14 (s, 2H), 5.58-5.59 (m, 1H), 6.04 (m, 1H), 6.27 (dd, 1H), 7.29-7.33 (m, 3H), 7.47-7.55 (m, 5H), 7.78-7.82 (m, 2H) ppm.

LRMS (ESI): m/z 641 [M+H]⁺

### Example 31

### (6alpha,11beta)-6,9-difluoro-11,21-dihydroxy-3,20-dioxopregna-1,4-dien-17-yl 4-(benzyloxy)benzoate

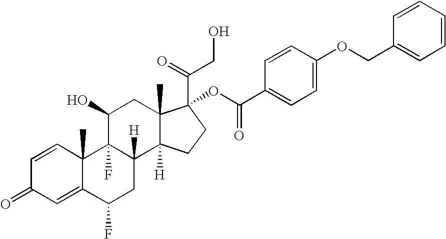

A suspension of (6alpha,11beta)-6,9-difluoro-11,17,21-trihydroxypregna-1,4-diene-3,20-dione (69 mg, 0.17 mmol) and 1-(benzyloxy)-4-(trimethoxymethyl)benzene as obtained in Preparation 66 (250 mg, 0.87 mmol) in toluene (2 mL) and 1,4-dioxane (1 mL) was treated with 4-methylbenzenesulfonic acid hydrate (10 mg, 50 μmol) and heated to 80° C. After 18 hours the solution was cooled to ambient temperature, diluted with water (10 mL) and extracted with ethyl acetate (10 mL). The organic phase was washed with brine (10 mL), dried (magnesium sulphate) and concentrated in vacuo. The residue was suspended in acetic acid (4 mL) and treated with water (100 μL). After stirring for 18 hours at ambient temperature the solution was diluted with water (10 mL) and extracted with ethyl acetate (10 mL). The organic phase was washed with brine (10 mL), dried (magnesium sulphate) and concentrated in vacuo. The residue was purified by flash column chromatography on silica gel eluting with heptane:ethyl acetate (19:1 to 1:4, by volume, gradient elution) to give the title compound as a white solid, 10 mg, 10% yield.

¹H NMR (400 MHz, DMSO-d₆) δ: 0.94 (s, 3H), 1.54 (s, 3H), 1.42-1.77 (m, 4H), 1.80-1.91 (m, 1H), 2.17-2.38 (m, 3H), 2.58-2.72 (m, 1H), 2.84-2.92 (m, 1H), 4.20-4.21 (m, 2H), 4.28-4.34 (m, 1H), 5.10 (t, 1H), 5.21 (s, 2H), 5.56-5.58 (m, 1H), 5.60-5.77 (m, 1H), 6.15 (m, 1H), 6.33-6.36 (m, 1H), 7.16-7.19 (m, 2H), 7.31-7.47 (m, 6H), 7.80-7.84 (m, 2H) ppm.

LRMS (ESI): m/z=607 [M+H]⁺

### Example 32

### (11beta)-9-fluoro-11,21-dihydroxy-3,20-dioxopregna-1,4-dien-17-yl 4-(benzyloxy)benzoate

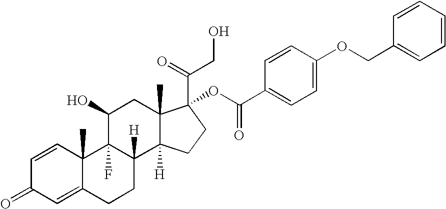

A suspension of (11beta)-9-fluoro-11,17,21-trihydroxypregna-1,4-diene-3,20-dione (181 mg, 0.48 mmol) and sodium sulphate (340 mg) in N,N-dimethylformamide (2 mL) was treated with 1-(benzyloxy)-4-(trimethoxymethyl)benzene as obtained in Preparation 66 (100 mg, 0.25 mmol) and 4-methylbenzenesulfonic acid hydrate (27 mg, 140 μmol) and heated to 80° C. Further 1-(benzyloxy)-4-(trimethoxymethyl)benzene (50 mg, 0.13 mmol) was added every 2 hours over a period of 8 hours. After 18 hours 4-methylbenzenesulfonic acid hydrate (5 mg, 26 μmol) was added and the solution was heated at 80° C. for 14 hours before the addition of 1-(benzyloxy)-4-(trimethoxymethyl)benzene (400 mg, 1.00 mmol). After heating at 80° C. for 13 hours 4-methylbenzenesulfonic acid hydrate (27 mg, 140 μmol) was added and the solution was heated at 80° C. for 15 hours. The Solution was cooled to ambient temperature, diluted with sodium hydrogen carbonate solution (2 mL, aqueous) and extracted with ethyl acetate (10 mL). The organic phase was dried (magnesium sulphate) and concentrated in vacuo. The residue was suspended in acetic acid (8 mL) and treated with water (400 μL). After stirring for 18 hours at ambient temperature the solution was diluted with water (20 mL) and extracted with ethyl acetate (3×20 mL). The organic phase was dried (magnesium sulphate) and concentrated in vacuo. The residue was purified by flash column chromatography on silica gel eluting with toluene:ethyl acetate (7:14, by volume) to give the title compound as a glass, 20 mg, 7% yield.

¹H NMR (400 MHz, CDCl₃) δ: 1.02 (s, 3H), 1.46-2.07 (m, 6H), 1.59 (s, 3H), 2.28-2.36 (m, 1H), 2.41-2.56 (m, 2H), 2.63-2.71 (m, 2H), 2.91-2.98 (m, 1H), 4.33-4.35 (m, 2H), 4.51-4.53 (m, 1H), 5.13 (s, 2H), 6.17 (m, 1H), 6.38-6.41 (m, 1H), 6.97-7.01 (m, 2H), 7.15-7.43 (m, 6H), 7.87-7.90 (m, 2H) ppm.

LRMS (ESI): m/z=589 [M+H]⁺

### Example 33

### cyanomethyl(11beta,17alpha)-9-fluoro-11-hydroxy-17-[(3-hydroxy-4-phenoxybenzoyl)oxy]-3-oxoandrosta-1,4-diene-17-carboxylate

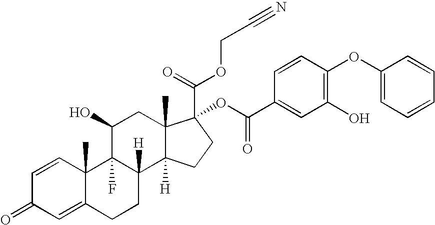

A solution of cyanomethyl(11beta,17alpha)-17-{[3-(benzyloxy)-4-phenoxybenzoyl]oxy}-9-fluoro-11-hydroxy-3-oxoandrosta-1,4-diene-17-carboxylate as obtained in Preparation 63 (50 mg, 71 μmol) in dichloromethane (5 mL, anhydrous) was cooled to −40° C. (acetonitrile/solid CO₂) and stirred under nitrogen before the dropwise addition of boron tribromide (1M solution in dichloromethane, 80 μL, 80 μmol). The reaction temperature was maintained at −40° C. for 3 hours before the reaction was quenched with methanol (5 mL) at −40° C. The reaction mixture was warmed to ambient temperature and diluted with dichloromethane (20 mL) and brine (20 mL). The aqueous phase was extracted with dichloromethane (2×20 mL). The combined organic extracts were dried (magnesium sulphate) and concentrated in vacuo. The residue was purified by flash column chromatography on silica gel eluting with dichloromethane:ethyl acetate (7:1, by volume) to give the title compound as a colourless solid, 27 mg, 62% yield.

¹H NMR (400 MHz, MeOD) δ: 1.18 (s, 3H), 1.67 (s, 3H), 1.57-1.70 (m, 2H), 1.81-1.89 (m, 2H), 2.00-2.08 (m, 2H), 2.28-2.36 (m, 1H), 2.45-2.51 (m, 2H), 2.57-2.72 (m, 1H), 2.77-2.86 (m, 1H), 3.02-3.09 (m, 1H), 4.40-4.44 (m, 1H), 4.94-4.95 (m, 2H), 6.15 (m, 1H), 6.34-6.37 (m, 1H), 6.90 (d, 1H), 7.01-7.04 (m, 2H), 7.14-7.18 (m, 1H), 7.36-7.48 (m, 4H), 7.54 (m, 1H) ppm.

LRMS (ESI): m/z 616 [M+H]⁺

### Examples 34-37

The following compounds of the general formula shown below were prepared by a method similar to that described for Example 33 by treatment of the appropriate starting material with boron tribromide in dichloromethane. The reactions were monitored by TLC or LCMS analysis.

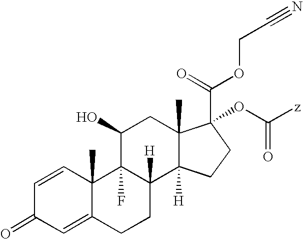

| No | Z | Name and NMR | Yield | LRMS |
|---|---|---|---|---|
| 34 | 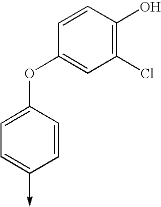 | cyanomethyl (11beta,17alpha)-17-{[4-(4- chloro-3-hydroxyphenoxy)-benzoyl]oxy}-9- fluoro-11-hydroxy-oxoandrosta-1,4-diene- 17-carboxylate Starting material: the compound as obtained in preparation 64. ¹H NMR (400 MHz, CDCl₃) δ: 1.13 (s, 3H), 1.49-1.85 (m, 4H), 1.59 (s, 3H), 1.92-2.07 (m, 2H), 2.25-2.33 (m, 1H), 2,40-2.71 (m, 4H), 3,01-3.09 (m, 1H), 4.49-4.52 (m, 1H), 4.66 (d, 1H), 4.93 (d, 1H), 6.16 (m, 1H), 6.37-6.40 (m, 1H), 6.57-6.60 (m, 1H), 6.72 (d, 1H), 6.98-7.02 (m, 2H), 7.22 (d, 1H), 7.31 (d, 1H), 7.89-7.93 (m, 2H) ppm. | 42% | (ESI): m/z 650 [M + H]⁺ |
| 35 | 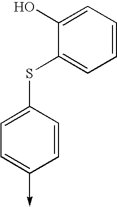 | cyanomethyl (11beta,17alpha)-9-fluoro-11- hydroxy-17-({4-[(2- hydroxyphenyl)thio]benzoyl}oxy)-3- oxoandrosta-1,4-diene-17-carboxylate Starting material: the compound as obtained in preparation 65. ¹H NMR (400 MHz, CDCl₃) δ: 1.10 (s, 3H), 1.46-1.65 (m, 2H), 1.57 (s, 3H), 1.71-1.79 (m, 2H), 1.87-2.04 (m, 2H), 2.19-2.27 (m, 1H), 2.38-2.55 (m, 3H), 2.61-2.69 (m, 1H), 2.97-3.04 (m, 1H), 4.44-4.46 (m, 1H), 4.63 (d, 1H), 4.85 (d, 1H), 6.14 (m, 1H), 6.33-6.36 (m, 1H), 6.64 (s, 1H), 6.95-6.99 (m, 1H), 7.03-7.09 (m, 3H), 7.24 (d, 1H), 7.38-7.43 (m, 1H), 7.47-7.49 (m, 1H), 7.75-7.78 (m, 2H) ppm. | 20% | (ESI): m/z 632 [M + H]⁺ |
| 36 | 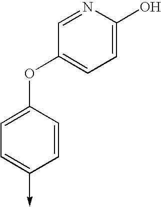 | cyanomethyl (11beta,17alpha)-9-fluoro-11- hydroxy-17-({4-[(6-hydroxypyridin-3- yl)oxy]benzoyl}-oxy)-3-oxoandrosta-1,4- diene-17-carboxylate Starting material: the compound as obtained in preparation 52. ¹H NMR (400 MHz, CDCl₃) δ: 1.13 (s, 3H), 1.49-1.68 (m, 2H), 1.59 (s, 3H), 1.74-1.84 (m, 2H), 1.91-2.07 (m, 2H), 2.24-2.32 (m, 1H), 2.40-2.57 (m, 3H), 2.63-2.71 (m, 1H), 3.01-3.08 (m, 1H), 4.48-4.52 (m, 1H), 4.67 (d, 1H), 4.90 (d, 1H), 6.16 (m, 1H), 6.37 (dd, 1H), 6.66 (d, 1H), 6.96-7.00 (m, 2H), 7.25-7.30 (m, 2H), 7.35-7.38 (m, 1H), 7.90-7.94 (m, 2H) ppm. | 29% | (ESI): m/z 617 [M + H]⁺ |
| 37 | 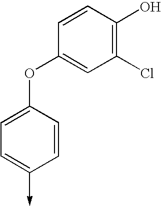 | cyanomethyl (11beta,17alpha)-17-{[4-(3- chloro-4-hydroxybhenoxy)-benzoyl]oxy}-9- fluoro-11-hydroxy-3-oxoandrosta-1,4-diene- 17-carboxylate Starting material: the compound as obtained in preparation 51. ¹H NMR (400 MHz, CDCl₃) δ: 1.13 (s, 3H), 1.49-1.73 (m, 3H), 1.59 (s, 3H), 1.76-1.84 (m, 1H), 1.91-2.07 (m, 2H), 2.24-2.33 (m, 1H), 2.39-2.71 (m, 4H), 3.01-3.08 (m, 1H), 4.48-4.53 (m, 1H), 4.65 (d, 1H), 4.92 (d, 1H), 5.61 (br s, 1H), 6.16 (m, 1H), 6.39 (dd, 1H), 6.90-6.96 (m, 3H), 7.03-7.07 (m, 2H), 7.23 (d, 1H), 7.88-7.91 (m, 2H) ppm. | 22% | (ESI): m/z 650 [M + H]⁺ |

### Example 38

### (11beta,17alpha)-17-{[(cyanomethyl)thio]carbonyl}-9-fluoro-11-hydroxy-3-oxoandrosta-1,4-dien-17-yl 4-[(4-chloro-3-hydroxyphenyl)thio]benzoate

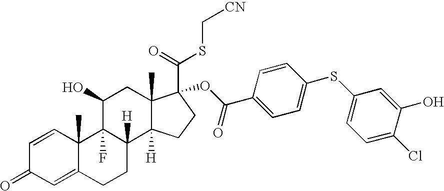

A solution of 4-[(3-acetoxy-4-chlorophenyl)thio]benzoic acid from preparation C (500 mg, 1.55 mmol) in N,N-dimethylformamide (6 mL) was cooled to 5° C. and treated with N-ethyl-N-isopropylpropan-2-amine (0.59 mL, 3.41 mmol) followed by o-(7-azabenzotriazol-1-yl)-N,N,N′,N′-tetramethyluronium hexafluorophosphate (648 mg, 1.70 mmol) portion wise. The solution was stirred under a nitrogen atmosphere for 15 minutes then (11beta,17alpha)-9-fluoro-11,17-dihydroxy-3-oxoandrosta-1,4-diene-17-carbothioic S-acid as obtained in Preparation 3 (589 mg, 1.55 mmol) was added portion wise. The reaction mixture was allowed to warm to ambient temperature and stirred for 15 hours. The mixture was then treated with N-ethyl-N-isopropylpropan-2-amine (0.30 mL, 1.70 mmol) followed by bromoacetonitrile (0.16 mL, 2.31 mmol). The reaction mixture was stirred at ambient temperature for a further 30 minutes and then treated with saturated aqueous sodium bicarbonate solution (10 mL) and methanol (20 mL). The mixture was stirred for a further 15 hours. The reaction mixture was then partitioned between ethyl acetate (100 mL) and hydrochloric acid (2N aqueous solution) (100 mL). The aqueous layer was extracted with ethyl acetate (1×50 mL) and the combined organic extracts dried (magnesium sulphate) and concentrated to dryness in vacuo. The residue was purified by flash column chromatography on silica gel eluting with heptanes:ethyl acetate (100:0 to 50:50, by volume, gradient elution). The afforded product was purified a second time by flash column chromatography on silica gel eluting with dichloromethane:ethyl acetate (100:0 to 70:30, by volume, gradient elution) to afford the title compound as a pale pink solid, 148 mg, 14% yield.

¹H NMR (400 MHz, DMSO-d₆) δ: 0.99 (s, 3H), 1.35-1.50 (m, 2H), 1.54 (s, 3H), 1.68-1.78 (m, 1H), 1.83-1.96 (m, 2H), 2.03-2.14 (m, 2H), 2.31-2.39 (m, 2H), 2.44-2.60 (m, 2H), 2.62-2.72 (m, 1H), 2.84-2.95 (m, 1H), 3.98-4.09 (m, 2H), 4.33 (bs, 1H), 5.61 (d, 1H), 6.05 (s, 1H), 6.28 (dd, 1H), 6.93 (dd, 1H), 7.05 (d, 1H), 7.31-7.38 (m, 2H), 7.44 (d, 1H), 7.82 (d, 2H), 10.54 (br s, 1H) ppm.

LRMS (ESI): m/z 682 [M+H]⁺

### Example 39

### cyanomethyl(11beta,17alpha)-17-({4-[(4-chloro-3-hydroxyphenyl)thio]benzoyl}oxy)-9-fluoro-11-hydroxy-3-oxoandrosta-1,4-diene-17-carboxylate

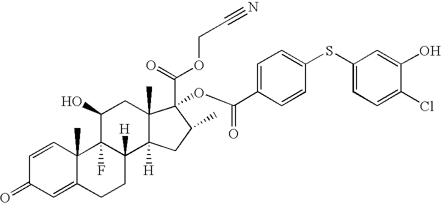

This compound of the general formula shown above was prepared by a similar method to that described for Example 38 using (11beta,17alpha)-9-fluoro-11,17-dihydroxy-3-oxoandrosta-1,4-diene-17-carboxylic acid (Phillipps et al, Journal of Medicinal Chemistry, 1994, pages 3717-3729) and 4-[(3-acetoxy-4-chlorophenyl)thio]benzoic acid as obtained in Preparation 75 as starting material in the presence of o-(7-azabenzotriazol-1-yl)-N,N,N′,N′-tetramethyluronium hexafluorophosphate. The reaction was monitored by TLC or LCMS analysis. When stated, purification was undertaken by flash chromatography on silica gel to afford the title compound as a white foam, 25% yield.

¹H NMR (400 MHz, DMSO-d₆) δ: 1.03 (s, 3H), 1.41-1.56 (m, 2H), 1.54 (s, 3H), 1.67-1.77 (m, 2H), 1.85-1.99 (m, 2H), 2.07-2.16 (m, 1H), 2.25-2.32 (m, 1H), 2.34-2.41 (m, 1H), 2.46-2.59 (m, 2H), 2.63-2.73 (m, 1H), 2.83-2.92 (m, 1H), 4.29 (bs, 1H), 5.06 (d, 2H), 5.57 (d, 1H), 6.05 (s, 1H), 6.28 (dd, 1H), 6.91 (dd, 1H), 7.03 (s, 1H), 7.33 (d, 1H), 7.36 (d, 2H), 7.43 (d, 1H), 7.80 (d, 1H), 10.52 (br s, 1H) ppm.

LRMS (ESI): m/z 666 [M+H]⁺

### Example 40

### (11beta,17alpha)-9-fluoro-17-{[(fluoromethyl)thio]carbonyl}-11-hydroxy-3-oxoandrosta-1,4-dien-17-yl 4-(4-chloro-3-hydroxyphenoxy)benzoate

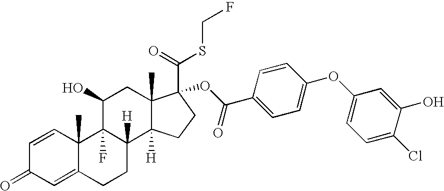

A solution of 4-(3-acetoxy-4-chlorophenoxy)benzoate as obtained in Preparation 78 (140 mg, 0.46 mmol) in N,N-dimethylformamide (2.5 mL) was cooled to 5° C. and treated with N-ethyl-N-isopropylpropan-2-amine (0.17 mL, 1.00 mmol) followed by o-(7-azabenzotriazol-1-yl)-N,N,N′,N′-tetramethyluronium hexafluorophosphate (191 mg, 0.50 mmol) portion wise. The solution was stirred under a nitrogen atmosphere for 30 minutes then (11beta,17alpha)-9-fluoro-11,17-dihydroxy-3-oxoandrosta-1,4-diene-17-carbothioic S-acid as obtained in Preparation 3 (174 mg, 0.46 mmol) was added portion wise. The reaction mixture was allowed to warm to ambient temperature and stirred for 1 hour and then treated with N-ethyl-N-isopropylpropan-2-amine (0.17 mL, 1.00 mmol) followed by a solution of bromofluoromethane (33% w/v solution in 2-butanone, 1.60 mL, 2.50 mmol). The reaction mixture was stirred at ambient temperature for 15 hours then partitioned between ethyl acetate (60 mL) and 2N hydrochloric acid (2N aqueous solution) (50 mL). The aqueous layer was extracted with ethyl acetate (2×30 mL) and the combined organic extracts dried (magnesium sulphate) and concentrated to dryness in vacuo. The residue was dissolved in methanol (5 mL) and was treated with saturated aqueous sodium bicarbonate solution (2 mL) and the reaction stirred for 45 minutes at ambient temperature. The solvents were removed in vacuo and the residue was partitioned between ethyl acetate (20 mL) and 2N hydrochloric acid (2N aqueous solution) (20 mL). The aqueous layer was extracted with ethyl acetate (2×20 mL) and the combined organic extracts dried (magnesium sulphate) and concentrated to dryness in vacuo. The residue was purified by flash column chromatography on silica gel eluting with dichloromethane:ethyl acetate (100:0 to 75:25, by volume, gradient elution) to afford the title compound as a white foam, 24 mg, 44% yield.

¹H NMR (400 MHz, DMSO-d₆) δ: 0.98 (s, 3H), 1.36-1.56 (m, 2H), 1.55 (s, 3H), 1.69-1.78 (m, 1H), 1.84-1.98 (m, 2H), 2.04-2.16 (m, 2H), 2.32-2.40 (m, 2H), 2.45-2.62 (m, 1H), 2.62-2.72 (m, 1H), 2.86-2.96 (m, 1H), 4.33 (bs, 1H), 5.55 (d, 1H), 5.86 (q, 1H), 5.98 (q, 1H), 6.05 (s, 1H), 6.28 (dd, 1H), 6.57 (dd, 1H), 6.67 (d, 1H), 7.17 (dt, 2H), 7.33 (d, 1H), 7.38 (d, 1H), 7.92 (dt, 2H), 10.46 (br s, 1H) ppm.

LRMS (ESI): m/z 659 [M+H]⁺657 [M−H]⁻

### Examples 41-42

The following compounds of the general formula shown below were prepared by a similar method to that described for Example 38 using 4-(3-acetoxy-4-chlorophenoxy)benzoic acid as obtained in Preparation 78, and the appropriate starting materials in the presence of o-(7-azabenzotriazol-1-yl)-N,N,N′,N′-tetramethyluronium hexafluorophosphate. The reactions were monitored by TLC or LCMS analysis. When stated, purification was undertaken by flash chromatography on silica gel.

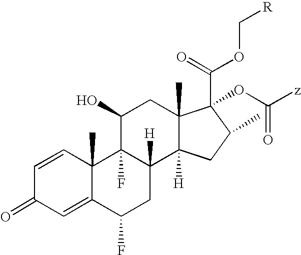

| No. | Z | Name and NMR | Yield | LRMS |
|---|---|---|---|---|
| 41 | 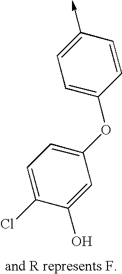 | Fluoromethyl (6alpha,11beta,16alpha,17alpha)-17- {[4-(4-chloro-3- hydroxyphenoxy)benzoyl]oxy}-6,9- difluoro-11-hydroxy-16-methyl-3- oxoandrosta-1,4-diene-17-carboxylate Starting material: (6alpha,11beta,16alpha,17alpha)-6,9- difluoro-11,17-dihydroxy-16-methyl-3- oxoandrosta-1,4-diene-17-carboxylic acid (Journal of Organic Chemistry (1986), 51(12), 2315-28). ¹H NMR (400 MHz, CDCl₃) δ: 0.99 (d, 3H), 1.16 (s, 3H), 1.25-1.39 (m, 4H), 1.55 (s, 3H), 1.77-1.95 (m, 4H), 2.31-2.51 (m, 5H), 3.39-3.48 (m, 1H), 4.39-4.47 (m, 1H), 5.32-5.49 (m, 2H), 5.58-5.80 (m, 1H), 5.98 (brd, 1H), 6.37-6.40 (m, 1H), 6.45 (s, 1H), 6.56-6.59 (m, 1H), 6.71-6.72 (m, 1H), 6.99-7.01 (m, 2H), 7.11-7.14 (m, 1H), 7.28-7.30 (m, 1H). 7.90-7.92 (m, 2H) ppm. | White solid, 10% (purified by semi-preparative HPLC) | (ESI): m/z 676 [M + H]⁺ |
| 42 | 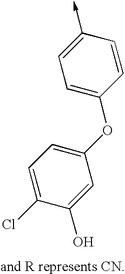 | Cyanomethyl (6alpha,11beta,16alpha,17alpha)-17- {[4-(4-chloro-3- hydroxyphenoxy)benzoyl]oxy}-6,9- difluoro-11-hydroxy-16-methyl-3- oxoandrosta-1,4-diene-17-carboxylate Starting materials: (6alpha,11beta,16alpha,17alpha)-6,9- difluoro-11,17-dihydroxy-16-methyl-3- oxoandrosta-1,4-diene-17-carboxylic acid (Journal of Organic Chemistry (1986), 51(12), 2315-28) with bromoacetonitrile as electrophile. ¹H NMR (400 MHz, CDCl₃) δ: 0.97 (d, 3H), 1.16 (s, 3H), 1.23-1.29 (m, 1H), 1.34-1.40 (m, 1H), 1.54 (s, 3H), 1.73-1.98 (m, 4H), 2.15-2.17 (m, 1H), 2.30-2.52 (m, 4H), 3.36-3.43 (m, 1H), 4.41 (d, 1H), 4.64 (d, 1H), 4.95 (d, 1H), 5.32-5.49 (m, 1H), 6.38-6.41 (m, 1H), 6.45 (s, 1H), 6.57 (dd, 1H), 6.71 (d, 1H), 7.00 (dt, 2H), 7.15 (dd, 1H), 7.29 (d, 1H), 7.89 (dt, 2H) ppm. | White solid 2% (purified by semi-preparative HPLC) | (ESI): m/z 683 [M + H]⁺ |

### Example 43

### (11beta,16alpha,17alpha)-9-fluoro-17-{[(fluoromethyl)thio]carbonyl}-11-hydroxy-16-methyl-3-oxoandrosta-1,4-dien-17-yl 4-(3-chloro-4-hydroxyphenoxy)benzoate

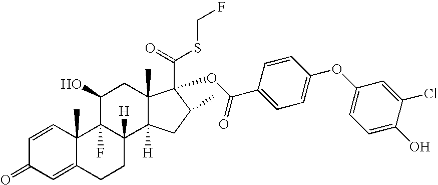

A solution of (11beta,16alpha,17alpha)-9-fluoro-17-{[(fluoromethyl)thio]carbonyl}-11-hydroxy-16-methyl-3-oxoandrosta-1,4-dien-17-yl as obtained in Preparation 70 (230 mg, 0.32 mmol) in methanol (10 mL) was treated with saturated aqueous sodium bicarbonate solution (2 mL) and the reaction stirred for 15 minutes at ambient temperature. A further 2 mL of saturated aqueous sodium bicarbonate solution was added and the mixture stirred for 15 minutes by which time the reaction had proceeded to completion. The solvents were removed in vacuo and the residue was partitioned between ethyl acetate (50 mL) and hydrochloric acid (2N aqueous solution) (50 mL). The aqueous layer was extracted with ethyl acetate (2×20 mL) and the combined organic extracts dried (magnesium sulphate) and concentrated to dryness in vacuo. The residue was purified by flash column chromatography on silica gel eluting with dichloromethane:ethyl acetate (100:0 to 80:20, by volume, gradient elution) to afford the title compound as a white foam, 187 mg, 86% yield.

¹H NMR (400 MHz, DMSO-d₆) δ: 0.94 (d, 3H), 1.10 (s, 3H), 1.26-1.35 (m, 1H), 1.42-1.52 (m, 1H), 1.53 (s, 3H), 1.83-2.00 (m, 3H), 2.17-2.30 (m, 2H), 2.34-2.42 (m, 1H), 2.42-2.58 (m, 1H), 2.62-2.73 (m, 1H), 3.36-3.45 (m, 1H), 4.29 (bs, 1H), 5.55 (d, 1H), 5.91 (s, 1H), 6.05 (d, 2H), 6.27 (dd, 1H), 6.97 (dd, 1H), 7.04 (d, 1H), 7.07 (dt, 2H), 7.20 (d, 1H), 7.32 (d, 1H) 7.87 (dt, 2H), 10.21 (br s, 1H) ppm.

LRMS (ESI): m/z 671 [M−H]⁻

### Example 44

### Fluoromethyl (6alpha,11beta,16alpha,17alpha)-17-{[4-(3-chloro-4-hydroxyphenoxy)benzoyl]oxy}-6,9-difluoro-11-hydroxy-16-methyl-3-oxoandrosta-1,4-diene-17-carboxylate

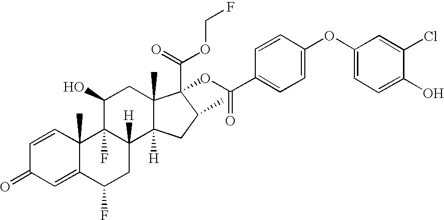

The following compound of the general formula shown below was prepared by a similar method to that described for Example 42 using fluoromethyl (6alpha,11beta,16alpha,17alpha)-17-{[4-(4-acetoxy-3-chlorophenoxy)benzoyl]oxy}-6,9-difluoro-11-hydroxy-16-methyl-3-oxoandrosta-1,4-diene-17-carboxylate as obtained in Preparation 80 as starting material in the presence of aqueous sodium bicarbonate solution. The reaction was monitored by TLC or LCMS analysis. When stated, purification was undertaken by flash chromatography on silica gel to afford the title compound as a white foam, 70% yield.

¹H NMR (400 MHz, DMSO-d₆) δ: 1.03 (s, 3H), 1.41-1.56 (m, 2H), 1.54 (s, 3H), 1.67-1.77 (m, 2H), 1.85-1.99 (m, 2H), 2.07-2.16 (m, 1H), 2.25-2.32 (m, 1H), 2.34-2.41 (m, 1H), 2.46-2.59 (m, 2H), 2.63-2.73 (m, 1H), 2.83-2.92 (m, 1H), 4.29 (bs, 1H), 5.06 (d, 2H), 5.57 (d, 1H), 6.05 (s, 1H), 6.28 (dd, 1H), 6.91 (dd, 1H), 7.03 (s, 1H), 7.33 (d, 1H), 7.36 (d, 2H), 7.43 (d, 1H), 7.80 (d, 1H), 10.52 (br s, 1H) ppm.

LRMS (ESI): m/z 673 [M−H]⁻

### Example 45

### cyanomethyl(6alpha,11beta,16alpha,17alpha)-17-{[4-(3-chloro-4-hydroxyphenoxy)benzoyl]oxy}-6,9-difluoro-11-hydroxy-16-methyl-3-oxoandrosta-1,4-diene-17-carboxylate

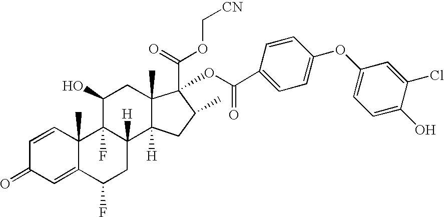

A solution of 4-(4-acetoxy-3-chlorophenoxy)benzoic acid as obtained in Preparation 33 (370 mg, 1.21 mmol) in N,N-dimethylformamide (6 mL) was cooled to 5° C. and treated with N-ethyl-N-isopropylpropan-2-amine (0.46 mL, 2.72 mmol) followed by o-(7-azabenzotriazol-1-yl)-N,N,N′,N′-tetramethyluronium hexafluorophosphate (530 mg, 1.39 mmol) portion wise. The solution was stirred under a nitrogen atmosphere for 30 minutes then (6alpha,11beta,16alpha,17alpha)-6,9-difluoro-11,17-dihydroxy-16-methyl-3-oxoandrosta-1,4-diene-17-carboxylic acid (Journal of Organic Chemistry (1986), 51(12), 2315-28) (480 mg, 1.21 mmol) was added portion wise. The reaction mixture was allowed to warm to ambient temperature and stirred for 1 hour. The mixture was then treated with N-ethyl-N-isopropylpropan-2-amine (0.46 mL, 2.72 mmol) followed by bromoacetonitrile (0.17 mL, 2.42 mmol). The reaction mixture was treated with further N-ethyl-N-isopropylpropan-2-amine (0.46 mL, 2.72 mmol) and bromoacetonitrile (0.17 mL, 2.42 mmol) and stirred at ambient temperature for a further 1 hour. The reaction mixture was then partitioned between ethyl acetate (40 mL) and hydrochloric acid (2N aqueous solution) (30 mL). The aqueous layer was extracted with ethyl acetate (2×30 mL) and the combined organic extracts dried (magnesium sulphate) and concentrated to dryness in vacuo. The residue was purified by flash column chromatography on silica gel eluting with dichloromethane:ethyl acetate (100:0 to 75:25, by volume, gradient elution) The afforded product was purified a second time by flash column chromatography on silica gel eluting with dichloromethane:ethyl acetate (100:0 to 75:25, by volume, gradient elution) to afford the title compound as a pale yellow foam, 80 mg, 5% yield.

¹H NMR (400 MHz, DMSO-d₆) δ: 0.89 (d, 3H), 1.08 (s, 3H), 1.27-1.35 (m, 1H), 1.53 (s, 3H), 1.53-1.67 (m, 1H), 1.71-1.78 (m, 1H), 1.86-1.98 (m, 1H), 2.13-2.21 (m, 1H), 2.23-2.35 (m, 2H), 2.52-2.70 (m, 1H), 3.28-3.38 (m, 1H), 4.22-4.29 (m, 1H), 5.06 (s, 2H), 5.57-5.77 (m, 1H), 5.67 (d, 1H), 6.15 (s, 1H), 6.33 (dd, 1H), 6.97 (dd, 1H), 7.01-7.09 (m, 3H), 7.19 (d, 1H), 7.30 (dd, 1H), 7.87 (dt, 2H), 10.23 (br s, 1H) ppm.

LRMS (ESI): m/z 682 [M+H] 680 [M−H]⁻

### Examples 46-49

The following compound of the general formula shown below was prepared by a similar method to that described for Example 45 using (6alpha,11beta,16alpha,17alpha)-6,9-difluoro-11,17-dihydroxy-16-methyl-3-oxoandrosta-1,4-diene-17-carboxylic acid (Journal of Organic Chemistry (1986), 51(12), 2315-28) and the appropriate starting materials in the presence of o-(7-azabenzotriazol-1-yl)-N,N,N′,N′-tetramethyluronium hexafluorophosphate. The reaction was monitored by TLC or LCMS analysis. When stated purification was undertaken by flash chromatography on silica gel.

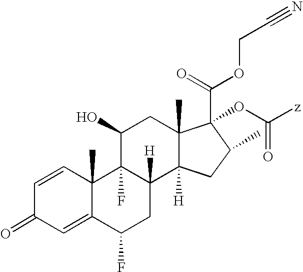

| No. | Z | Name and NMR | Yield | LRMS |
|---|---|---|---|---|
| 46 | 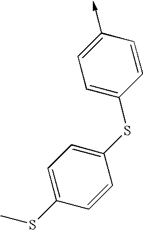 | Cyanomethyl (6alpha,11beta,16alpha,17alpha)-6,9- difluoro-11-hydroxy-16-methyl-17-[(4-{[4- (methylthio)phenyl]thio}benzoyl)oxy]-3- oxoandrosta-1,4-diene-17-carboxylate Starting material: 4-{[4- (methylthio)phenyl]thio}benzoic acid as obtained in Preparation 83 ¹H NMR (400 MHz, DMSO-d₆) δ: 0.82-0.84 (m, 3H), 1.03 (s, 3H), 1.22-1.27 (m, 3H), 1.48 (s, 3H), 1.54-1.60 (m, 1H), 1.67-1.71 (m, 1H), 1.82-1.92 (m, 1H), 2.09-2.12 (m, 1H), 2.19-2.30 (2H, m), 2.25-2.66 (m, 1H), 3.25-3.27 (m, 1H), 4.20- 4.22 (m, 1H), 5.02 (s, 2H), 5.55-5.71 (m, 2H), 6.10 (m, 1H), 6.30 (dd, 1H), 7.21-7.27 (m, 3H), 7.31-7.34 (m, 2H), 7.41-7.45 (m, 2H), 7.72-7.75 (m, 2H) ppm. | Yellow solid 6% (purified) | (ESI): m/z 694 [M + H]⁺ |
| 47 | 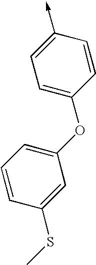 | Cyanomethyl (6alpha,11beta,16alpha, 17alpha)-6,9-difluoro-11-hydroxy-16- methyl-17-({4-[3-(methylthio)phenoxy] benzoyl}oxy)-3-oxoandrosta-1,4-diene-17- carboxylate Starting material: 4-[3-(methylthio) phenoxy]benzoic acid as obtained in Preparation 84. ¹H NMR (400 MHz, DMSO-d₆) δ: 0.85-0.87 (m, 3H), 1.04 (s, 3H), 1.22-1.31 (m, 4H), 1.49 (s, 3H), 1.57 (t, 1H), 1.71 (t, 1H), 1.88 (q, 1H), 2.12-2.16 (m, 1H), 2.22-2.30 (m, 2H), 2.44 (s, 3H), 2.54-2.60 (m,1H), 3.28-3.30 (m, 4H), 4.21-4.24 (m, 1H), 5.03 (s, 2H), 5.55-5.72 (m, 2H), 6.10 (s, 1H), 6.30 (dd, 1H), 6.82-6.85 (m, 1H), 6.98 (t, 1H), 7.07-7.11 (m, 3H), 7.26 (dd, 1H), 7.34 (t, 1H), 7.85-7.86 (m, 2H) ppm. | white solid 43% (purified) | (ESI): m/z 678 [M + H]⁺ |
| 48 | 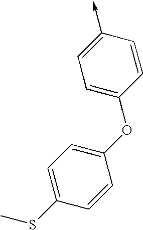 | Cyanomethyl (6alpha,11beta,16alpha, 17alpha)-6,9-difluoro-11-hydroxy-16- methyl-17-({4-[4-(methylthio)phenoxy] benzoyl}oxy)-3-oxoandrosta-1,4-diene-17- carboxylate Starting material: 4-[4-(methylthio) phenoxy]benzoic acid as obtained in Preparation 82. ¹H NMR (400 MHz, DMSO-d₆) δ: 0.86 (d, 3H), 1.04 (s, 3H), 1.22-1.30 (m, 3H), 1.49 (s, 3H),1.58 (q, 1H), 1.71 (d, 1H), 1.87 (q, 1H), 2.11-2.16 (m, 1H), 2.21-2.29 (m, 2H), 2.54 -2.64 (m, 1H), 3.26-3.31 (m, 3H), 4.19- 4.24 (m, 1H), 5.03 (d, 2H), 5.54 -5.71 (m, 2H), 6.10 (s, 1H), 6.29 (dd, 1H), 7.04-7.07 (m, 3H), 7.26 (dd, 1H), 7.29-7.33 (m, 2H), 7.83-7.86 (m, 2H) ppm. | white solid 5% (purified) | (ESI): m/z 678 [M + H]⁺ |
| 49 | 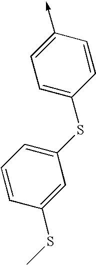 | Cyanomethyl (6alpha,11beta,16alpha, 17alpha)-6,9-difluoro-11-hydroxy-16- methyl-17-[(4-{[3-(methylthio)phenyl]thio} benzoyl)oxy]-3-oxoandrosta-1,4-diene-17- carboxylate Starting material: 4-{[3- (methylthio)phenyl]thio}benzoic acid as obtained in Preparation 41. ¹H NMR (400 MHz, DMSO-d₆) δ: 0.82-0.85 (m, 3H), 1.03 (s, 3H), 1.22-1.30 (m, 3H), 1.48 (s, 3H), 1.57 (t, 1H), 1.69 (d, 1H), 1.87 (q, 1H), 2.10-2.16 (1H, m), 2.20-2.28 (m, 2H), 2.45 (s, 3H), 2.54-2.63 (m, 1H), 3.25-3.32 (m, 1H), 4.19-4.23 (m, 1H), 5.02 (s, 2H), 5.55-5.52 (m, 2H), 6.10 (s, 1H), 6.29 (dd, 1H), 7.21-7.24 (m, 2H), 7.26-7.33 (m, 4H), 7.36-7.40 (m, 1H), 7.75-7.78 (m, 2H) ppm. | white solid 71% (purified) | (ESI): m/z 694 [M + H]⁺ |

### Example 50-53

The following compounds of the general formula shown below were prepared by a similar method to that described for Example 45 using (6alpha,11beta,16alpha,17alpha)-6,9-difluoro-11,17-dihydroxy-16-methyl-3-oxoandrosta-1,4-diene-17-carboxylic acid (Journal of Organic Chemistry (1986), 51(12), 2315-28) and the appropriate starting materials in the presence of o-(7-azabenzotriazol-1-yl)-N,N,N′,N′-tetramethyluronium hexafluorophosphate. Alkylation was achieved using a solution of bromofluoromethane (33% v/v solution in 2-butanone). The reaction was monitored by TLC or LCMS analysis. When stated, purification was undertaken by semi preparative HPLC on a small amount of the crude material, so recovered yields do not reflect crude reaction yield.

Conditions of semi-preparative HPLC: the column is a phenomenex luna 5 micrometre column. Packed with a C18 100 angstrom core. Dimensions=150×21.2. The detection is set at 254 nm and the PC is running trilution 2.1 software controlling a Gilson system (liquid handler and pump). The gradient used in all cases is as follows: 0-2.5 mins=95% Aqueous (0.05% formic acid in water). 2.5-17.5 mins=95% Aq to 95° A organic (0.05% formic acid in acetonitrile). 17.5-22.5 min=95% organic.

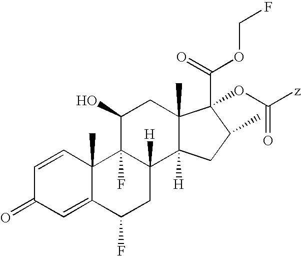

| No. | Z | Name and NMR | Yield | LRMS |
|---|---|---|---|---|
| 50 | 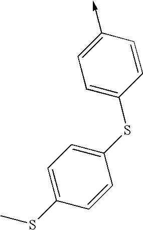 | Fluoromethyl (6alpha,11beta,16alpha, 17alpha)-6,9-difluoro-11-hydroxy-16- methyl-17-[(4-{[4-(methylthio)phenyl]thio} benzoyl)oxy]-3-oxoandrosta-1,4-diene-17- carboxylate Starting material: 4-{[4-(methylthio)phenyl] thio}benzoic acid as obtained in Prep. 83. ¹H NMR (400 MHz, CDCl₃) δ: 0.97 (d, 3H), 1.15 (s, 3H), 1.26-1.37 (m, 3H), 1.50 (t, 1H), 1.55 (s, 3H), 1.77-1.94 (m, 4H), 2.29-2.50 (m, 7H), 3.39-3.48 (m, 1H), 4.41-4.46 (m, 1H), 5.32-5.48 (m, 1H), 5.44 -6.07 (m, 3H), 6.44 (dd, 1H), 6.46 (t, 1H), 7.10-7.14 (m, 3H), 7.24-7.28 (m, 2H), 7.39-7.41 (m, 2H), 7.75-7.77 (m, 2H) ppm. | white solid 14% (purified) | (ESI): m/z 687 [M + H]⁺ |
| 51 | 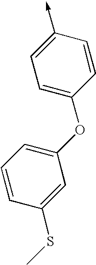 | Fluoromethyl (6alpha,11beta,16alpha, 17alpha)-6,9-difluoro-11-hydroxy-16- methyl-17-({4-[3-(methylthio) phenoxy] benzoyl}oxy)- 3-oxoandrosta-1,4-diene-17- carboxylate Starting material: 4-[3-(methylthio)phenoxy] benzoic acid as obtained in Preparation 84. ¹H NMR (400 MHz, CDCl₃) δ: 0.99 (d, 3H), 1.16 (s, 3H), 1.26-1.39 (m, 2H), 1.55 (s, 3H), 1.77-1.94 (m, 4H), 2.32-2.50 (m, 6H), 3.41-3.48 (m, 1H), 4.41-4.47 (m, 1H), 5.32-5.49 (m, 2H), 5.48-6.07 (m, 2H), 6.38 (dd, 1H), 6.43-6.45 (m, 1H), 6.87-6.80 (m, 1H), 6.91-6.92 (m, 1H), 6.97-6.99 (m, 2H), 7.04-7.06 (m, 1H), 7.13-7.16 (m, 1H), 7.25-7.29 (m, 1H), 7.89-7.91 (m, 2H) ppm. | white solid 8% (purified) | (ESI): m/z 671 [M + H]⁺ |
| 52 | 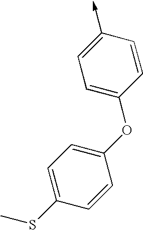 | Fluoromethyl (6alpha,11beta,16alpha, 17alpha)-6,9-difluoro-11-hydroxy-16-methyl- 17-({4-[4-(methylthio)phenoxy]benzoyl}oxy)- 3-oxoandrosta-1,4-diene-17- carboxylate Starting material: 4-[4-(methylthio)phenoxy] benzoic acid as obtained in Preparation 82. ¹H NMR (400 MHz, CDCl₃) δ: 0.99 (d, 3H), 1.16 (s, 3H), 1.26-1.33 (m, 2H), 1.54 (s, 3H), 1.76-1.94 (m, 4H), 2.29-2.50 (m, 6H), 3.40-3.48 (m, 1H), 4.40-4.46 (m, 1H), 5.32-5.49 (m, 2H), 5.44 -5.57 (d, 1H), 5.94 -6.07 (d, 1H), 6.39 (dd, 1H), 6.45 (s, 1H), 6.95-6.99 (m, 3H), 7.11-7.14 (m, 1H), 7.26 -7.29 (m, 2H), 7.88-7.90 (m, 2H) ppm. | white solid 6% (purified) | (ESI): m/z 671 [M + H]⁺ |
| 53 | 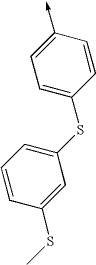 | Fluoromethyl (6alpha,11beta,16alpha, 17alpha)-6,9-difluoro-11-hydroxy-16-methyl- 17-({4-[4-(methylthio)phenoxy]benzoyl}oxy)- 3-oxoandrosta-1,4-diene-17- carboxylate Starting material: 4-{[3- (methylthio)phenyl]thio}benzoic acid as obtained in Preparation 41. ¹H NMR (400 MHz, CDCl₃) δ: 0.97 (d, 3H), 1.15 (s, 3H), 1.26-1.38 (m, 2H), 1.54 (s, 3H), 1.76-1.94 (m, 4H), 2.28-2.52 (m, 7H), 3.39-3.48 (m, 1H), 4.40-4.46 (m, 1H), 5.32-5.49 (m, 2H), 5.44-5.57 (m, 1H), 5.98 (br d, 1H), 6.37-6.40 (m, 1H), 6.45 (s, 1H), 7.13-7.16 (m, 1H), 7.17-7.20 (m, 2H), 7.21-7.26 (m, 2H), 7.28-7.34 (m, 2H), 7.77-7.80 (m, 2H) ppm. | white solid 14% (purified) | (ESI): m/z 687 [M + H]⁺ |

The following compounds may also be prepared using similar procedures as those described above:
(11beta, 17alpha)-9-fluoro-17-{[(fluoromethyl)thio]carbonyl}-11-hydroxy-3-oxoandrosta-1,4-dien-17-yl 4-[(4-chloro-3-hydroxyphenyl)thio]benzoate;
(11beta,17alpha)-17-{[(cyanomethyl)thio]carbonyl}-9-fluoro-11-hydroxy-3-oxoandrosta-1,4-dien-17-yl 4-(4-chloro-3-hydroxyphenoxy)benzoate;
cyanomethyl(11beta, 17alpha)-17-[(4-{[(3-chloro-4-hydroxyphenyl)thio]methyl}benzoyl)oxy]-9-fluoro-11-hydroxy-3-oxoandrosta-1,4-diene-17-carboxylate;
(11beta, 17alpha)-9-fluoro-17-{[(fluoromethyl)thio]carbonyl}-11-hydroxy-3-oxoandrosta-1,4-dien-17-yl 4-{[(3-chloro-4-hydroxyphenyl)thio]methyl}benzoate;
(11beta, 17alpha)-17-{[(cyanomethyl)thio]carbonyl}-9-fluoro-11-hydroxy-3-oxoandrosta-1,4-dien-17-yl 4-{[(3-chloro-4-hydroxyphenyl)thio]methyl}benzoate;
cyanomethyl(11beta, 17alpha)-9-fluoro-11-hydroxy-17-[({6-[(6-hydroxypyridin-3-yl)oxy]pyridin-3-yl}carbonyl)oxy]-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 6-[(6-hydroxypyridin-3-yl)oxy]nicotinate;
(11beta,17alpha)-9-fluoro-17-{[(fluoromethyl)thio]carbonyl}-11-hydroxy-3-oxoandrosta-1,4-dien-17-yl 6-[(6-hydroxypyridin-3-yl)oxy]nicotinate;
cyanomethyl(11beta,17alpha)-9-fluoro-11-hydroxy-17-({4-[(6-hydroxypyridazin-3-yl)oxy]benzoyl}oxy)-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-[(6-hydroxypyridazin-3-yl)oxy]benzoate;
(11beta,17alpha)-9-fluoro-17-{[(fluoromethyl)thio]carbonyl}-11-hydroxy-3-oxoandrosta-1,4-dien-17-yl 4-[(6-hydroxypyridazin-3-yl)oxy]benzoate;
cyanomethyl(11beta,17alpha)-9-fluoro-11-hydroxy-17-({4-[(5-hydroxypyrazin-2-yl)oxy]benzoyl}oxy)-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-[(5-hydroxypyrazin-2-yl)oxy]benzoate;
(11beta,17alpha)-9-fluoro-17-{[(fluoromethyl)thio]carbonyl}-11-hydroxy-3-oxoandrosta-1,4-dien-17-yl 4-[(5-hydroxypyrazin-2-yl)oxy]benzoate;
cyanomethyl(11beta,17alpha)-17-{[4-(3-cyano-4-hydroxyphenoxy)benzoyl]oxy}-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-(3-cyano-4-hydroxyphenoxy)benzoate;
(11beta,17alpha)-9-fluoro-17-{[(fluoromethyl)thio]carbonyl}-11-hydroxy-3-oxoandrosta-1,4-dien-17-yl 4-(3-cyano-4-hydroxyphenoxy)benzoate
cyanomethyl(11beta,17alpha)-17-{[4-(4-cyano-3-hydroxyphenoxy)benzoyl]oxy}-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl-4-(4-cyano-3-hydroxyphenoxy)benzoate;
(11beta,17alpha)-9-fluoro-17-{[fluoromethyl)thio]carbonyl}-11-hydroxy-3-oxoandrosta-1,4-dien-17-yl 4-(4-cyano-3-hydroxyphenoxy)benzoate;
cyanomethyl(11beta,17alpha)-17-{[4-(3-carbamoyl-4-hydroxyphenoxy)benzoyl]oxy}-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-(3-carbamoyl-4-hydroxyphenoxy)benzoate;
(11beta,17alpha)-9-fluoro-17-{[fluoromethyl)thio]carbonyl}-11-hydroxy-3-oxoandrosta-1,4-dien-17-yl 4-(3-carbamoyl-4-hydroxyphenoxy)benzoate;
cyanomethyl(11beta,17alpha)-17-{[4-(4-carbamoyl-3-hydroxyphenoxy)benzoyl]oxy}-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-(4-carbamoyl-3-hydroxyphenoxy)benzoate;
(11beta,17alpha)-9-fluoro-17-{[(fluoromethyl)thio]carbonyl}-11-hydroxy-3-oxoandrosta-1,4-dien-17-yl 4-(4-carbamoyl-3-hydroxyphenoxy)benzoate;
cyanomethyl(11beta,17alpha)-17-({4-[3-(dimethylcarbamoyl)-4-hydroxyphenoxy]benzoyl}oxy)-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-[3-(dimethylcarbamoyl)-4-hydroxyphenoxy]benzoate;
(11beta,17alpha)-9-fluoro-17-{[(fluoromethyl)thio]carbonyl}-11-hydroxy-3-oxoandrosta-1,4-dien-17-yl-4-[3-(dimethylcarbamoyl)-4-hydroxyphenoxy]benzoate,
cyanomethyl(11beta,17alpha)-17-({4-[4-(dimethylcarbamoyl)-3-hydroxyphenoxy]benzoyl}oxy)-9-fluoro-11-hydroxy-3-oxoandrosta-1,4-diene-17-carboxylate,
(11beta,17alpha)-17-{[(cyanomethyl)thio]carbonyl}-9-fluoro-11-hydroxy-3-oxoandrosta-1,4-dien-17-yl-4-[4-(dimethylcarbamoyl)-3-hydroxyphenoxy]benzoate; and
(11beta, 17alpha)-9-fluoro-17-{[(fluoromethyl)thio]carbonyl}-11-hydroxy-3-oxoandrosta-1,4-dien-17-yl 4-[4-(dimethylcarbamoyl)-3-hydroxyphenoxy]benzoate.

### In Vitro Pharmacological Activity

The pharmacological activity of the compounds of formula (I) was assessed in in vitro assays of glucocorticoid agonist activity and in isolated leukocyte TNF-α release assays which are predictive of anti-inflammatory activity in vivo.

Glucococorticoid receptor (GR) agonist potency was determined in the human chondrosarcoma cell-line SW1353 stably transfected with an MMTV-luciferase reporter construct. SW1353 naturally expresses human GR, which on binding a glucocorticoid agonist activates glucocorticoid response elements within the MMTV promoter, driving expression of the luciferase gene.

Frozen SW1353 cells were revived in DMEM medium, without sodium pyruvate or phenol red, supplemented with 2 mM L-glutamine, 1 μg/ml insulin, 2 mg/ml lactalbumin hydrosylate and 0.5 μg/ml ascorbate. Cells were seeded at approximately 5000 cells/well (35 μl/well) in 384-well clear bottom, tissue culture treated plates. Steroid dose-response dilutions were prepared in steroid diluent (PBS containing 2.5% (v/v) DMSO and 0.05% (v/v) pluronic detergent) and 5 μl added to each well. The volume was made up to 50 μl per well with steroid diluent. Positive control wells contained 1 μM dexamethasone. Plates were incubated for approximately 18 hours at 37° C. in an air/5% CO₂ atmosphere in a humidified incubator before Britelite reagent (10 μl; Perkin-Elmer) was added to each well. Each plate was incubated for 2 minutes in the dark and luminescence quantified using a LJL Biosystems Analyst luminometer. Data for test compounds (expressed as percentage of the dexamethasone positive control) were used to construct dose response curves from which EC₅₀ values were estimated. The following data have been obtained:

| | GR agonist |
|---|---|
| Example No. | EC₅₀ (nM) |
| 1 | 22.3 |
| 2 | 115 |
| 3 | 50.5 |
| 4 | 23.6 |
| 5 | 33.3 |
| 6 | 23.5 |
| 7 | 39.9 |
| 8 | 25.0 |
| 9 | n/a* |
| 10 | 62.1 |
| 11 | 30.2 |
| 12 | 117 |
| 13 | 80.9 |
| 14 | 23.9 |
| 15 | 35.1 |
| 16 | 24.0 |
| 17 | 37.9 |
| 18 | 34.9 |
| 19 | 44.8 |
| 20 | 39.2 |
| 21 | 49.0 |
| 22 | 69.9 |
| 23 | 52.5 |
| 24 | 25.8 |
| 25 | 19.8 |
| 26 | 42.6 |
| 27 | 43.3 |
| 28 | 37.3 |
| 29 | 78.4 |
| 30 | 20.6 |
| 31 | 102 |
| 32 | 36.0 |
| 33 | 68.3 |
| 34 | 100 |
| 35 | 75.2 |
| 36 | 16.3 |
| 37 | 39.7 |
| 38 | 54.7 |
| 39 | 44.1 |
| 40 | 31.2 |
| 41 | 7.73 |
| 42 | 24.4 |
| 43 | 26.4 |
| 44 | 6.70 |
| 45 | 21.3 |
| 46 | 44.3 |
| 47 | 25.2 |
| 48 | 14.2 |
| 49 | 90.8 |
| 50 | 11.5 |
| 51 | 8.70 |
| 52 | 6.05 |
| 53 | 11.8 |

*n/a = not available

The anti-inflammatory activity of the compounds against human leukocytes in vitro was also evaluated by determining inhibition of tumour necrosis factor-α (TNF-α) release from lipopolysaccharide (LPS) stimulated isolated human peripheral mononuclear cells (PBMC).

Peripheral venous blood from healthy, non-medicated donors was collected using ethylenediaminetetraacetic acid (EDTA) as the anti-coagulant. For PBMC preparation, samples of blood were diluted 1:1 with sterile phosphate buffered saline and then separated using ACCUSPIN™ System-Histopaque®-1077 tubes (Sigma-Aldrich, St Louis, Mo.), centrifuged at 400 g for 35 minutes. Buffy coat cells were removed into PBS, centrifuged at 200 g for 10 minutes and re-suspended in PBMC assay buffer (Hanks Balanced Salt Solution, 0.28% [w/v] 4-[2-hydroxyethyl]-1-piperazineethanesulfonic acid [HEPES], 0.01% [w/v] low-endotoxin bovine serum albumin [BSA]. A differential white cell count was performed and PBMC's diluted to 1×10⁶ lymphocytes per ml in PBMC assay buffer.

Test compounds were dissolved in DMSO and diluted in PBMC assay buffer (final DMSO concentration 1%) to cover an appropriate concentration range, e.g 0.001 nM to 10000 nM. Samples of test compound solution or vehicle (20 μl) were added into 96-well tissue culture treated plates (Corning) and PBMC (160 μl) added to each well. The assay mixtures were incubated at 37° C. for 1 h in a humidified incubator containing an atmosphere of air supplemented with 5% CO₂ before adding LPS (20 μl of 100 ng/ml for PBMC). Plates were returned to the incubator for a further 18 hours, and then centrifuged before recovery of samples of supernatant. TNF-α in the samples was determined using an enzyme-linked immunosorbent assay (ELISA) (Invitrogen kit no CHC-1754; Invitrogen Carlsbad, Calif.) and following the manufacturers instructions. Dose response curves were constructed from which IC₅₀ values were calculated. The following data have been obtained:

| | IC₅₀ (nM) for |
|---|---|
| | inhibition of TNF-α |
| Example No. | release |
| 1 | 0.095 |
| 2 | 0.373 |
| 3 | 0.143 |
| 4 | 0.154 |
| 5 | 0.152 |
| 6 | 0.207 |
| 7 | 0.555 |
| 8 | 0.292 |
| 9 | 0.210 |
| 10 | 0.826 |
| 11 | 0.735 |
| 12 | 0.345 |
| 13 | 0.799 |
| 14 | 0.234 |
| 15 | 0.164 |
| 16 | 0.067 |
| 17 | 0.157 |
| 18 | 0.138 |
| 19 | 0.300 |
| 20 | 0.290 |
| 21 | 0.656 |
| 22 | 0.087 |
| 23 | 0.536 |
| 24 | 0.117 |
| 25 | 0.026 |
| 26 | 0.177 |
| 27 | 0.326 |
| 28 | 0.231 |
| 29 | 0.156 |
| 30 | 0.256 |
| 31 | 0.152 |
| 32 | 0.506 |
| 33 | 0.212 |
| 34 | 0.278 |
| 35 | 0.099 |
| 36 | 0.167 |
| 37 | 0.201 |
| 38 | 0.755 |
| 39 | 0.615 |
| 40 | 0.053 |
| 41 | 0.032 |
| 42 | 0.443 |
| 43 | 0.060 |
| 44 | 0.029 |
| 45 | 0.155 |
| 46 | 0.372 |
| 47 | 0.731 |
| 48 | 0.090 |
| 49 | 0.738 |
| 50 | 0.052 |
| 51 | 0.057 |
| 52 | 0.033 |
| 53 | 0.086 |

### In Vivo Pharmacological Activity

The pharmacological activity may be assessed in in vivo models of lung inflammation such as the one described below. The primary objective of this procedure was to determine the anti-inflammatory activity of the compounds of formula (I), when administered directly into the lungs via the trachea.

Test compounds are dissolved, or prepared as fine suspensions, in phosphate buffered saline containing 0.5% (w/v>Tween-80 to provide a range of dose levels. Male CD Sprague-Dawley rats (300-450 g) are randomised to study groups of n=6 and then briefly anaesthetised in an anaesthetic chamber with 5% Isoflurane in 3 l/min O₂. One of the test compound formulations or dose vehicle (100 μl) is injected directly into the trachea of each anaesthetised rat using a Hamilton syringe. The animals are then allowed to recover from the anaesthetic. Dependent on the study design, animals receive either a single dose of compound or are treated once daily on 4 successive days. Four hours after the dosing (or 4 hours after the final dose in repeat dose studies) the rats are placed into a chamber (300×300×450 mm), connected to an ultrasonic nebuliser and a small animal rodent ventilator set to the maximum tidal volume and rate (5 ml, 160 strokes/min). 10 ml of 1 mg/ml LPG (Sigma-Aldrich, L2630) dissolved in saline, pre-warmed to 37° C., is nebulised into the chamber. After 15 minutes the ventilator and nebuliser are turned off and the animals remain in the chamber to breathe the mist for a further 15 minutes before being returned to the home cage.

Four hours after the end of the LPS treatment the animals are terminally anaesthetised with 1 ml/kg Pentoject IP. The trachea is cannulated and the lungs lavaged with 4×2.5 ml PBS containing 2.6 mM EDTA and the lavage fluid collected. 1 ml bronchioalveolar lavage (BAL) is added to 125 μl of 40% bovine serum albumen (BSA) and the cellular count determined using an Advia 120 haematology system (Siemens). In repeat dose studies, a terminal blood sample is collected from each rat, plasma prepared, and concentrations of corticosterone in serum and ACTH in plasma determined. The corticosterone and ACTH levels, together with changes in bodyweight, and weights of dissected adrenal and thymus glands are used to assess systemic glucocorticoid agonist effects. In some studies a known glucocorticoid agonist, fluticasone propionate, is administered to separate groups of rats as a positive control.

Separate dose response curves are constructed, for inhibition of LPS-induced lung neutrophils, and each marker of systemic glucocorticoid agonist effect. Half maximal effect doses (ED₅₀) values are estimated from the fitted curves.

1. A compound of formula (I): 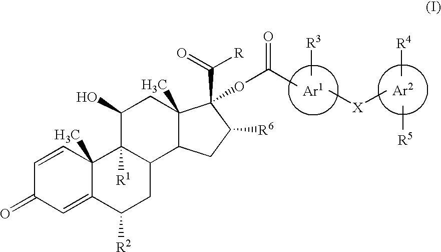
or a pharmaceutically acceptable salt thereof, wherein:
R¹ and R² are independently of each other selected from H, F, Cl and methyl;
R is selected from —CH₂—OH, —O—CH₂—CN, —S—CH₂—CN, —O—CH₂F, —S—CH₂F, —O—CH₂Cl and —S—CH₂Cl;
X is a direct bond or is selected from —O—, —S—, —CH₂—S—, —S—CH₂—, —CH₂—, —O—CH₂, and —CH₂—O—;
Ar¹ is a phenyl or a pyridine;
Ar² is an aryl group selected from phenyl, pyridine, pyridazine, pyrazine and pyrimidine;
R³ is H or OH;
R⁴ is H or OH; and
R⁵ is selected from H, CN, halogen, (C₁-C₄)alkyl, —S—(C₁-C₄)alkyl, —CONR⁷R⁸, —SO₂NR⁷R⁸ and NHSO₂CH₃;
R⁶ is H or CH₃; and
R⁷ and R⁸ are the same or are different and are independently selected from H and (C₁-C₄)alkyl.

2. A compound according to claim 1, or a pharmaceutically acceptable salt thereof, wherein R¹ is F and R² is H or F.

3. A compound according to claim 1, or a pharmaceutically acceptable salt thereof, wherein R is —O—CH₂F.

4. A compound according to claim 1, or a pharmaceutically acceptable salt thereof, wherein Ar¹ and Ar² are both phenyl.

5. A compound according to claim 1, or a pharmaceutically acceptable salt thereof, wherein R³ is H.

6. A compound according to claim 1, or a pharmaceutically acceptable salt thereof, wherein R⁴ is OH.

7. A compound according to claim 6, or a pharmaceutically acceptable salt thereof, wherein said OH group is in a meta or para position relative to X.

8. A compound according to claim 1, or a pharmaceutically acceptable salt thereof, wherein R⁵ is H, Cl or —S—CH₃.

9. A compound according to claim 1, or a pharmaceutically acceptable salt thereof, wherein X is —O—.

10. A compound according to claim 1, or a pharmaceutically acceptable salt thereof, of formula (Ia): 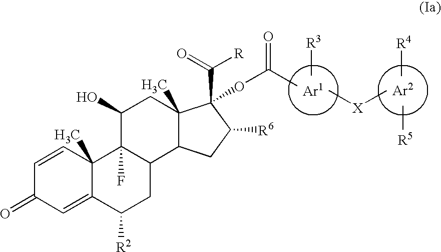
wherein:
R² is H or F;
R is selected from —CH₂—OH, —O—CH₂—CN, —S—CH₂—CN, —O—CH₂F, —S—CH₂F, —O—CH₂Cl and —S—CH₂Cl;
X is a direct bond or is selected from —O—, —S—, —CH₂—S—, —S—CH₂—, —CH₂—, —O—CH₂, and —CH₂—O—;
Ar¹ is a phenyl or a pyridine;
Ar² is an aryl group selected from phenyl, pyridine, pyridazine, pyrazine and pyrimidine;
R³ is H or OH;
R⁴ is H or OH; and
R⁵ is selected from H, CN, halogen, (C₁-C₄)alkyl, —S—(C₁-C₄)alkyl, —CONR⁷R⁸, —SO₂NR⁷R⁸ and NHSO₂CH₃;
R⁶ is H or CH₃; and
R⁷ and R⁸ are the same or are different and are independently selected from H and (C₁-C₄)alkyl.

11. A compound according to claim 1, or a pharmaceutically acceptable salt thereof, of formula (Ib): 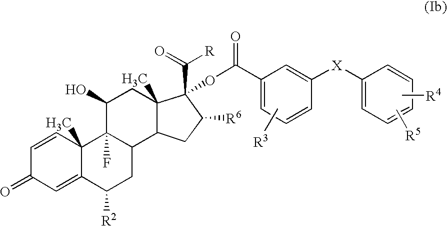
wherein:
R² is H or F;
R is selected from —CH₂—OH, —O—CH₂—CN, —S—CH₂—CN, —S—CH₂F and —S—CH₂Cl;
X is a direct bond or is selected from —O—, —S—, —CH₂—S—, —S—CH₂—, —CH₂— and —O—CH₂;
R³ is H or OH;
R⁴ is H or OH;
R⁵ is H, Cl or —S—CH₃; and
R⁶ is H or CH₃.

12. A compound according to claim 1, or a pharmaceutically acceptable salt thereof or a pharmaceutically acceptable solvate of said compound or salt, of formula (Ic): 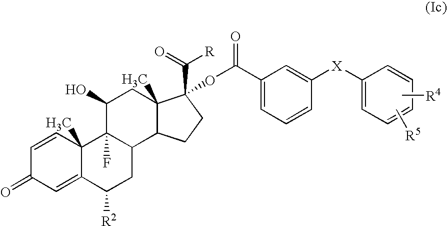
wherein:
R² is H or F;
R is selected from —CH₂—OH, —O—CH₂—CN, —S—CH₂—CN, —S—CH₂F and —S—CH₂Cl;
X is a direct bond or is selected from —O—, —S—, —CH₂—S—, —S—CH₂—, —CH₂— and —O—CH₂;
R⁴ is H or OH; and
R⁵ is H or Cl.

13. A compound according to claim 1, or a pharmaceutically acceptable salt thereof, of formula (Id): 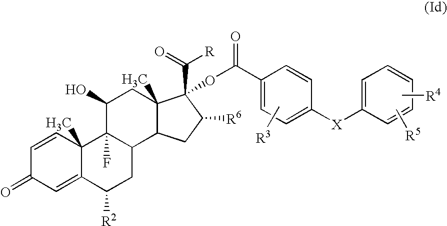
wherein:
R² is H or F;
R is selected from —CH₂—OH, —O—CH₂—CN, —S—CH₂—CN, —S—CH₂F, —O—CH₂F and —S—CH₂Cl;
X is a direct bond or is selected from —O—, —S—, —CH₂—S—, —S—CH₂—, —CH₂— and —O—CH₂;
R³ is H or OH;
R⁴ is H or OH;
R⁵ is H, Cl or —S—CH₃; and
R⁶ is H or CH₃.

14. A compound according to claim 1, or a pharmaceutically acceptable salt thereof, of formula (Ie): 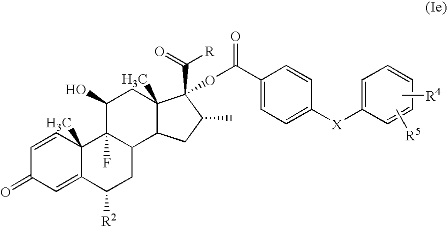
wherein:
R² is H or F;
R is selected from —CH₂—OH, —O—CH₂—CN, —S—CH₂—CN, —S—CH₂F, —O—CH₂F and —S—CH₂Cl;
X is a direct bond or is selected from —O—, —S—, —CH₂—S—, —S—CH₂—, —CH₂— and —O—CH₂;
R⁴ is H or OH; and
R⁵ is H or Cl.

15. A compound according to claim 1, or a pharmaceutically acceptable salt thereof, of formula (If): 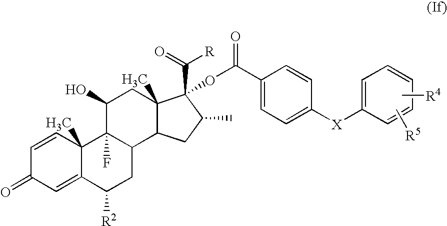
wherein:
R² is H or F;
R is selected from —O—CH₂—CN, —S—CH₂—CN, —S—CH₂F and —O—CH₂F;
X is a direct bond or is selected from —O— and —S—;
R⁴ is OH; and
R⁵ is Cl.

16. A compound according to claim 1, or a pharmaceutically acceptable salt thereof, of formula (Ig): 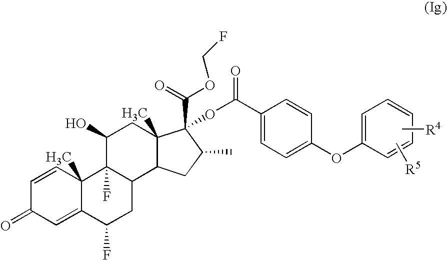
wherein R⁴ is OH and R⁵ is Cl.

17. A compound according to claim 1, or a pharmaceutically acceptable salt thereof, which is selected from:
cyanomethyl(6alpha,11beta,17alpha)-17-[(4-benzylbenzoyl)oxy]-6,9-difluoro-11-hydroxy-3-oxoandrosta-1,4-diene-17-carboxylate;
cyanomethyl(6alpha,11beta,17alpha)-17-[(biphenyl-4-ylcarbonyl)oxy]-6,9-difluoro-11-hydroxy-3-oxoandrosta-1,4-diene-17-carboxylate;
cyanomethyl(6alpha,11beta,17alpha)-6,9-difluoro-11-hydroxy-3-oxo-17-{[4-(phenylthio)benzoyl]oxy}androsta-1,4-diene-17-carboxylate;
cyanomethyl(6alpha,11beta,17alpha)-6,9-difluoro-11-hydroxy-3-oxo-17-[(4-phenoxybenzoyl)oxy]androsta-1,4-diene-17-carboxylate;
cyanomethyl(6alpha,11beta,17alpha)-6,9-difluoro-11-hydroxy-3-oxo-17-({4-[(phenylthio)methyl]benzoyl}oxy)androsta-1,4-diene-17-carboxylate;
cyanomethyl(6alpha,11beta,17alpha)-6,9-difluoro-11-hydroxy-17-({4-[(4-hydroxybenzyl)thio]benzoyl}oxy)-3-oxoandrosta-1,4-diene-17-carboxylate;
cyanomethyl(11beta,17alpha)-17-[(biphenyl-4-ylcarbonyl)oxy]-9-fluoro-11-hydroxy-3-oxoandrosta-1,4-diene-17-carboxylate;
cyanomethyl(11beta,17alpha)-9-fluoro-11-hydroxy-3-oxo-17-{[4-(phenylthio)benzoyl]oxy}androsta-1,4-diene-17-carboxylate;
cyanomethyl(11beta,17alpha)-17-[(4-benzylbenzoyl)oxy]-9-fluoro-11-hydroxy-3-oxoandrosta-1,4-diene-17-carboxylate;
cyanomethyl(11beta,17alpha)-9-fluoro-11-hydroxy-17-[(4-{[3-(methylthio)-phenyl]thio}benzoyl)oxy]-3-oxoandrosta-1,4-diene-17-carboxylate;
cyanomethyl(6alpha,11beta,17alpha)-6,9-difluoro-11-hydroxy-17-[(4-{[(4-hydroxyphenyl)thio]methyl}benzoyl)oxy]-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-(3-chloro-4-hydroxyphenoxy)benzoate;
(11beta,17alpha)-17-{[(cyanomethyl)thio]carbonyl}-9-fluoro-11-hydroxy-3-oxoandrosta-1,4-dien-17-yl 4-[(3-chloro-4-hydroxyphenyl)thio]benzoate;
cyanomethyl(11beta,17alpha)-9-fluoro-11-hydroxy-3-oxo-17-[(4-phenoxybenzoyl)oxy]androsta-1,4-diene-17-carboxylate;
cyanomethyl(11beta,17alpha)-9-fluoro-11-hydroxy-17-({4-[(3-hydroxyphenyl)thio]-benzoyl}oxy)-3-oxoandrosta-1,4-diene-17-carboxylate;
cyanomethyl(11beta,17alpha)-9-fluoro-11-hydroxy-17-{[4-(4-hydroxyphenoxy)benzoyl]oxy}-3-oxoandrosta-1,4-diene-17-carboxylate;
cyanomethyl(11beta,17alpha)-9-fluoro-11-hydroxy-17-{[4-(3-hydroxyphenoxy)benzoyl]oxy}-3-oxoandrosta-1,4-diene-17-carboxylate;
cyanomethyl(11beta,17alpha)-9-fluoro-11-hydroxy-17-({4-[(4-hydroxyphenyl)thio]benzoyl}oxy)-3-oxoandrosta-1,4-diene-17-carboxylate;
cyanomethyl(11beta,17alpha)-9-fluoro-11-hydroxy-17-{[3-hydroxy-4-(phenylthio)benzoyl]oxy}-3-oxoandrosta-1,4-diene-17-carboxylate;
cyanomethyl(11beta,17alpha)-17-({4-[(3-chloro-4-hydroxyphenyl)thio]-benzoyl}oxy)-9-fluoro-11-hydroxy-3-oxoandrosta-1,4-diene-17-carboxylate;
cyanomethyl(11beta,17alpha)-9-fluoro-11-hydroxy-17-[(2-hydroxy-4-phenoxybenzoyl)oxy]-3-oxoandrosta-1,4-diene-17-carboxylate;
(11beta,17alpha)-9-fluoro-17-{[(fluoromethyl)thio]carbonyl}-11-hydroxy-3-oxoandrosta-1,4-dien-17-yl 4-benzylbenzoate;
(11beta,17alpha)-9-fluoro-17-{[(fluoromethyl)thio]carbonyl}-11-hydroxy-3-oxoandrosta-1,4-dien-17-yl 4-{[3-(methylthio)phenyl]thio}benzoate;
(11beta,17alpha)-9-fluoro-17-{[(fluoromethyl)thio]carbonyl}-11-hydroxy-3-oxoandrosta-1,4-dien-17-yl 4-(phenylthio)benzoate;
(11beta,17alpha)-9-fluoro-17-{[(fluoromethyl)thio]carbonyl}-11-hydroxy-3-oxoandrosta-1,4-dien-17-yl 4-phenoxybenzoate;
(11beta,17alpha)-9-fluoro-17-{[(fluoromethyl)thio]carbonyl}-11-hydroxy-3-oxoandrosta-1,4-dien-17-yl 4-(3-chloro-4-hydroxyphenoxy)-benzoate;
(11beta,17alpha)-9-fluoro-17-{[fluoromethyl)thio]carbonyl}-11-hydroxy-3-oxoandrosta-1,4-dien-17-yl 4-[(3-chloro-4-hydroxyphenyl)thio]benzoate;
(11beta,17alpha)-17-{[(cyanomethyl)thio]carbonyl}-9-fluoro-11-hydroxy-3-oxoandrosta-1,4-dien-17-yl 4-(phenylthio)benzoate;
(11beta,17alpha)-17-{[(cyanomethyl)-thio]carbonyl}-9-fluoro-11-hydroxy-3-oxoandrosta-1,4-dien-17-yl 4-phenoxybenzoate;
(11beta,17alpha)-17-{[(chloromethyl)thio]carbonyl}-9-fluoro-11-hydroxy-3-oxoandrosta-1,4-dien-17-yl-4-(phenylthio)benzoate;
(6alpha,11beta)-6,9-difluoro-11,21-dihydroxy-3,20-dioxopregna-1,4-dien-17-yl-4-(benzyloxy)benzoate;
(11beta)-9-fluoro-11,21-dihydroxy-3,20-dioxopregna-1,4-dien-17-yl 4-(benzyloxy)benzoate;
cyanomethyl(11beta,17alpha)-9-fluoro-11-hydroxy-17-[(3-hydroxy-4-phenoxybenzoyl)oxy]-3-oxoandrosta-1,4-diene-17-carboxylate;
cyanomethyl(11beta,17alpha)-17-{[4-(4-chloro-3-hydroxyphenoxy)-benzoyl]oxy}-9-fluoro-11-hydroxy-oxoandrosta-1,4-diene-17-carboxylate;
cyanomethyl(11beta,17alpha)-9-fluoro-11-hydroxy-17-({4-[(2-hydroxyphenyl)thio]benzoyl}oxy)-3-oxoandrosta-1,4-diene-17-carboxylate;
cyanomethyl(11beta,17alpha)-9-fluoro-11-hydroxy-17-({4-[(6-hydroxypyridin-3-yl)oxy]benzoyl}-oxy)-3-oxoandrosta-1,4-diene-17-carboxylate;
cyanomethyl(11beta,17alpha)-17-{[4-(3-chloro-4-hydroxyphenoxy)-benzoyl]oxy}-9-fluoro-11-hydroxy-3-oxoandrosta-1,4-diene-17-carboxylate;
(11beta,17alpha)-17-{[(cyanomethyl)thio]carbonyl}-9-fluoro-11-hydroxy-3-oxoandrosta-1,4-dien-17-yl 4-[(4-chloro-3-hydroxyphenyl)thio]benzoate;
Cyanomethyl(11beta,17alpha)-17-({4-[(4-chloro-3-hydroxyphenyl)thio]benzoyl}oxy)-9-fluoro-11-hydroxy-3-oxoandrosta-1,4-diene-17-carboxylate;
(11beta,17alpha)-9-fluoro-17-{[(fluoromethyl)thio]carbonyl}-11-hydroxy-3-oxoandrosta-1,4-dien-17-yl 4-(4-chloro-3-hydroxyphenoxy)benzoate;
Fluoromethyl (6alpha,11beta,16alpha,17alpha)-17-{[4-(4-chloro-3-hydroxyphenoxy)benzoyl]oxy}-6,9-difluoro-11-hydroxy-16-methyl-3-oxoandrosta-1,4-diene-17-carboxylate;
Cyanomethyl(6alpha,11beta,16alpha,17alpha)-17-{[4-(4-chloro-3-hydroxyphenoxy)benzoyl]oxy}-6,9-difluoro-11-hydroxy-16-methyl-3-oxoandrosta-1,4-diene-17-carboxylate;
(11beta,16alpha,17alpha)-9-fluoro-17-{[(fluoromethyl)thio]carbonyl}-11-hydroxy-16-methyl-3-oxoandrosta-1,4-dien-17-yl 4-(3-chloro-4-hydroxyphenoxy)benzoate;
Fluoromethyl (6alpha,11beta,16alpha,17alpha)-17-{[4-(3-chloro-4-hydroxyphenoxy)benzoyl]oxy}-6,9-difluoro-11-hydroxy-16-methyl-3-oxoandrosta-1,4-diene-17-carboxylate;
cyanomethyl(6alpha, 11beta,16alpha,17alpha)-17-{[4-(3-chloro-4-hydroxyphenoxy)benzoyl]oxy}-6,9-difluoro-11-hydroxy-16-methyl-3-oxoandrosta-1,4-diene-17-carboxylate;
Cyanomethyl(6alpha,11beta,16alpha,17alpha)-6,9-difluoro-11-hydroxy-16-methyl-17-[(4-{[4-(methylthio)phenyl]thio}benzoyl)oxy]-3-oxoandrosta-1,4-diene-17-carboxylate;
Cyanomethyl(6alpha,11beta,16alpha, 17alpha)-6,9-difluoro-11-hydroxy-16-methyl-17-({4-[3-(methylthio)phenoxy]benzoyl}oxy)-3-oxoandrosta-1,4-diene-17-carboxylate;
Cyanomethyl(6alpha,11beta,16alpha, 17alpha)-6,9-difluoro-11-hydroxy-16-methyl-17-({-4-[4-(methylthio)phenoxy]benzoyl}oxy)-3-oxoandrosta-1,4-diene-17-carboxylate;
Cyanomethyl(6alpha,11beta,16alpha, 17alpha)-6,9-difluoro-11-hydroxy-16-methyl-17-[(4-{[3-(methylthio)phenyl]thio}benzoyl)oxy]-3-oxoandrosta-1,4-diene-17-carboxylate;
Fluoromethyl (6alpha,11beta,16alpha, 17alpha)-6,9-difluoro-11-hydroxy-16-methyl-17-[(4-{[4-(methylthio)phenyl]thio}benzoyl)oxy]-3-oxoandrosta-1,4-diene-17-carboxylate;
Fluoromethyl (6alpha,11beta,16alpha, 17alpha)-6,9-difluoro-11-hydroxy-16-methyl-17-({4-[3-(methylthio)phenoxy]benzoyl}oxy)-3-oxoandrosta-1,4-diene-17-carboxylate;
Fluoromethyl (6alpha,11beta,16alpha, 17alpha)-6,9-difluoro-11-hydroxy-16-methyl-17-({-4-[4-(methylthio)phenoxy]benzoyl}oxy)-3-oxoandrosta-1,4-diene-17-carboxylate; and
Fluoromethyl (6alpha,11beta,16alpha, 17alpha)-6,9-difluoro-11-hydroxy-16-methyl-17-({4-[4-(methylthio)phenoxy]benzoyl}oxy)-3-oxoandrosta-1,4-diene-17-carboxylate.

18. A compound according to claim 1, or a pharmaceutically acceptable salt thereof, which is cyanomethyl(11beta,17alpha)-17-{[4-(4-chloro-3-hydroxyphenoxy)-benzoyl]oxy}-9-fluoro-11-hydroxy-oxoandrosta-1,4-diene-17-carboxylate.

19. A compound according to claim 1, or a pharmaceutically acceptable salt thereof, which is fluoromethyl (6alpha,11beta,16alpha,17alpha)-17-{[4-(3-chloro-4-hydroxyphenoxy)benzoyl]oxy}-6,9-difluoro-11-hydroxy-16-methyl-3-oxoandrosta-1,4-diene-17-carboxylate.

20. A compound according to claim 1, or a pharmaceutically acceptable salt thereof, which is fluoromethyl (6alpha,11beta,16alpha,17alpha)-17-{[4-(4-chloro-3-hydroxyphenoxy)benzoyl]oxy}-6,9-difluoro-11-hydroxy-16-methyl-3-oxoandrosta-1,4-diene-17-carboxylate.

21. A pharmaceutical composition comprising an effective amount of a compound of claim 1, or a pharmaceutically acceptable salt thereof, and one or more pharmaceutically acceptable excipients.

22. A pharmaceutical composition according to claim 21 which further comprises one or several other therapeutic agent(s) selected from:
(a) 5-Lipoxygenase (5-LO) inhibitors or 5-lipoxygenase activating protein (FLAP) antagonists;
(b) Leukotriene antagonists (LTRAs) including antagonists of LTB₄, LTC₄, LTD₄, and LTE₄;
(c) Inhibitors of leukotriene C4 synthase;
(d) Histamine receptor antagonists including H1, H3 and H4 antagonists;
(e) α₁- and α₂-adrenoceptor agonist vasoconstrictor sympathomimetic agents for decongestant use;
(f) PDE inhibitors, including PDE3, PDE4 and PDE5 inhibitors;
(g) Theophylline;
(h) Sodium cromoglycate;
(i) COX inhibitors selected from both non-selective and selective COX-1 or COX-2 inhibitors (NSAIDs);
(j) Prostaglandin receptor antagonists and inhibitors of prostaglandin synthase, including hPGDS;
(k) Muscarinic M3 receptor antagonists or anticholinergic agents;
(l) β2-adrenoceptor agonists;
(m) Monoclonal antibodies active against endogenous proinflammatory entities, including IgE, IL3, IL4, IL9, IL10, IL13, IL17A, GMCSF and their receptors;
(n) Anti-tumor necrosis factor (anti-TNF-α) agents;
(o) Adhesion molecule inhibitors including VLA-4 antagonists;
(p) Kinin-B₁- and B₂-receptor antagonists;
(q) Immunosuppressive agents, including inhibitors of the IgE pathway and cyclosporine;
(r) Inhibitors of matrix metalloproteases (MMPs) including MMP9 and MMP12;
(s) Tachykinin NK₁, NK₂ and NK₃ receptor antagonists;
(t) Protease inhibitors such as elastase inhibitors including neutrophil elastase inhibitors;
(u) Adenosine A2a receptor agonists and A2b antagonists;
(v) Inhibitors of urokinase;
(w) Compounds that act on dopamine receptors including D2 agonists;
(x) Modulators of the NFκβ pathway including IKK inhibitors;
(y) modulators of cytokine signalling pathyways including p38 MAP kinase, PI3 kinases, JAK kinases, syk kinase, EGFR, MK-2, fyn kinases or ITK;
(z) Agents that can be classed as mucolytics or anti-tussive;
(aa) Agents, which enhance or re-sensitise responses to inhaled corticosteroids such as macolide analogues and inhibitors of PI3Kδ or AKT1,2,3;
(bb) Antibiotics and antiviral agents effective against micro-organisms which can colonise the respiratory tract;
(cc) HDAC activators;
(dd) CXCR1, CXCR2 and CXCR3 antagonists;
(ee) Integrin antagonists;
(ff) Chemokines and chemokine receptor antagonists;
(gg) Epithelial sodium channel (ENaC) blockers or Epithelial sodium channel (ENaC) inhibitors;
(hh) CRAC ion channel blockers or CRAC inhibitors;
(ii) P2Y2 Agonists and other Nucleotide receptor agonists;
(jj) P2X7 antagonists;
(kk) Inhibitors of VAP1;
(ll) Inhibitors of thromboxane;
(mm) Niacin; and
(nn) Adhesion factors including VLAM, ICAM, and ELAM.

23. A pharmaceutical composition according to claim 21 which further comprises a 32-adrenoceptor agonist and/or an anticholinergic agent.

24. A method of treating a disease, disorder or condition involving the glucocorticoid receptor in a mammal, the method comprising administering to said mammal in need thereof an effective amount of a compound of claim 1 or a pharmaceutically acceptable salt thereof, said disease, disorder or condition being selected from the group consisting of skin diseases; inflammatory conditions of the nose, throat or lungs; inflammatory conditions of the intestine; auto-immune diseases; and ocular conditions.

25. The method of claim 24 wherein said skin disease is eczema, psoriasis, dermatitis, pruritis or a hypersensitivity reaction.

26. The method of claim 24 wherein said inflammatory condition of the nose, throat or lungs is rhinitis, sinusitis, asthma, nasal polyps, chronic obstructive pulmonary disease (COPD) or fibrosis.

27. The method of claim 24 wherein said inflammatory disease of the intestine is inflammatory bowel disease, Crohn's disease or ulcerative colitis.

28. The method of claim 24 wherein said auto-immune disease is rheumatoid arthritis.

29. The method of claim 24 wherein said ocular condition is conjunctivitis.

30. A compound of formula (IV): 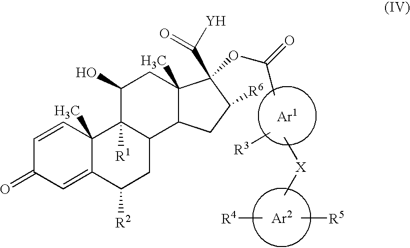
wherein
R¹ and R² are independently of each other selected from H, F, Cl and methyl;
Y is O or S;
X is a direct bond or is selected from —O—, —S—, —CH₂—S—, —S—CH₂—, —CH₂—, —O—CH₂, and —CH₂—O—;
Ar¹ is a phenyl or a pyridine;
Ar² is an aryl group selected from phenyl, pyridine, pyridazine, pyrazine and pyrimidine;
R³ is H or OH;
R⁴ is H or OH; and
R⁵ is selected from H, CN, halogen, (C₁-C₄)alkyl, —S—(C₁-C₄)alkyl, —CONR⁷R⁸, —SO₂NR⁷R⁸ and NHSO₂CH₃;
R⁶ is H or CH₃; and
R⁷ and R⁸ are the same or are different and are independently selected from H and (C₁-C₄)alkyl.
